(19) United States
(12) Patent Application Publication
(10) Pub. No.: US 2013/0315959 A1
(43) Pub. Date: Nov. 28, 2013

(54) COMPOUNDS

(71) Applicants: Paolo Costantino, Siena (IT); Roberto Adamo, Siena (IT); Maria Rosaria Romano, Siena (IT); Elisa Danieli, Siena (IT); Francesco Berti, Siena (IT); Emilia Cappelliti, Siena (IT); Luigi Lay, Siena (IT)

(72) Inventors: Paolo Costantino, Siena (IT); Roberto Adamo, Siena (IT); Maria Rosaria Romano, Siena (IT); Elisa Danieli, Siena (IT); Francesco Berti, Siena (IT); Emilia Cappelliti, Siena (IT); Luigi Lay, Siena (IT)

(73) Assignee: NOVARTIS AG, Basel (CH)

(21) Appl. No.: 13/997,017

(22) PCT Filed: Dec. 23, 2011
(86) PCT No.: PCT/IB2011/003244

§ 371 Date: Aug. 13, 2013

(30) Foreign Application Priority Data
Dec. 24, 2010 (GB) 1022042.4
Jul. 4, 2011 (GB) 1111440.2

(51) Int. Cl. A61K 39/08

(52) U.S. Cl. CPC A61K 39/08
USPC 424/247.1; 536/55.1

(57) ABSTRACT

The invention provides a synthetic C. difficile PS-II cell wall saccharide. The invention also provides a process for purifying C. difficile PS-II saccharide from C. difficile bacterial cells resulting in reduced contamination. The saccharides may be used in vaccines, particularly as conjugates with carrier proteins.

### TECHNICAL FIELD

This invention is in the field of bacterial saccharides, particularly those of Clostridium difficile, and particularly for use in the preparation of vaccines. This invention also relates to methods of purifying bacterial saccharides.

### BACKGROUND ART

Saccharides from bacteria have been used for many years in vaccines against bacteria. As saccharides are T-independent antigens, however, they are poorly immunogenic. Conjugation to a carrier can convert T-independent antigens into T-dependent antigens, thereby enhancing memory responses and allowing protective immunity to develop. The most effective saccharide vaccines are therefore based on glycoconjugates, and the prototype conjugate vaccine was against Haemophilus influenzae type b (‘Hib’) [e.g. chapter 14 of ref. 86].

Another bacterium for which conjugate vaccines have been proposed is Clostridium difficile (C. difficile). C. difficile is a Gram positive spore-forming anaerobic bacterium, which is considered the most important definable cause of nosocomial diarrhea (refs. 1 and 2).

Since its description in 1978 as a cause of antimicrobial-associated diarrhea, colitis and pseudomembranous colitis (PMC), the interest in this pathogen has grown due to its impact on morbidity and mortality in the elderly and among hospitalized patients [3]. The incidence of C. difficile infection (CDI) is rapidly increasing in the US and Canada [4], where a recent study reported an incidence of 22.5 cases per 1,000 hospital admissions, which was associated with a significantly high mortality rate of 6.9% (refs. 3 and 5).

The most virulent strain is generally considered to be the ribotype 027 or North American pulsotype 1 (NAP1, or BI/NAP1/027), which caused outbreaks in 16 European countries in 2008 [2]. Current treatment modalities for CDI are suboptimal, with up to 20% of treated patients failing to respond to antibiotics and relapses occurring in up to 25% of additional cases after initial clinical resolution [6].

Treatment failures and recurrences with antibiotics are emphasizing the need for the discovery of new preventative agents using vaccination either based on protein or carbohydrate antigens (refs. 7, 8, and 9). CDI is a toxin-mediated disease, and the major virulence factors studied are two exotoxins, toxin A and toxin B. In addition, several other factors may play a role in disease manifestation, including a binary toxin (CDT), molecules facilitating adhesion, capsule production and hydrolytic enzyme secretion (ref. 8). Therapeutic treatment of CDI infection is based on two different antibiotics (metronidazole and oral vancomycin), but there are disadvantages associated with this antibiotic approach to treatment, namely antibiotic resistance, increasing recurrence rates and emergent hypervirulent strains. Investigations are underway into whether C. difficile polysaccharides could be considered as vaccine candidates.

Monteiro's group recently analyzed the cell wall saccharide of C. difficile ribotype 027 and two additional strains, MOH900 (classified as NAP2) and MOH718 [10]. Two different structures were identified, named PS-I and PS-II. PS-II is the only structure occurring in most C. difficile strains, suggesting that PS-II may be a conserved surface antigen. The PS-II cell wall saccharide is composed of a hexasaccharide phosphate repeating unit:

[→6)-β-D-Glcp-(1→3)-D-GalpNAc-(1→4)-α-D-Glcp-(1→4)-β-D-Glcp-(1→3]-β-D-GalpNAc-(1→3)-α-D-Manp-(1→P]

Monteiro isolated PS-II by growing bacterial cells of C. difficile ribotype 027 in C. difficile Moxalactam Norfloxacin (CDMN) broth and subsequently inactivating the cells with sodium hypochlorite and isolating the carbohydrates from the bacterial cell surface using acetic acid [10]. The saccharide preparation obtained from the acid treatment of C. difficile ribotype 027 was subjected to size exclusion chromatography and further anion exchange chromatography [10].

The present inventors have found that PS-II isolated from C. difficile bacterial cells may be contaminated with other bacterial components. This contamination is undesirable, particularly when the saccharide is for medical use. There is therefore a need for further or improved processes for purifying C. difficile PS-II saccharides which result in less contamination. There is also a need for a synthetic route to the saccharides which provide well-defined structures without contamination with bacterial components.

### DISCLOSURE OF THE INVENTION

The inventors have produced C. difficile PS-II saccharides with reduced contamination. These saccharides are particularly suitable for use in medicines, e.g. in vaccines.

In a first aspect, the invention provides a synthetic C. difficile PS-II cell wall saccharide. A synthetic product eliminates the need for fermentation and isolation of bacteria, yielding saccharides with low contamination. For example, the synthetic saccharide may have low peptidoglycan contamination, optionally no peptidoglycan contamination. A synthetic C. difficile PS-II cell wall saccharide may also contain less protein contamination, optionally no protein contamination.

In a second aspect, the invention provides a process for purifying C. difficile PS-II saccharide from C. difficile bacterial cells. The process comprises a step of inactivating the bacterial cells by treatment with acid, preferably acetic acid. Advantageously, the inactivation step may also result in release of the saccharide from the cells. The inactivation step is followed by one or more optional processing steps such as fractionation, e.g. to remove protein contaminants; enzymatic treatment, e.g. to remove nucleic acid, protein and/or peptidoglycan contaminants; anion exchange chromatography, e.g. to remove residual protein; concentration using tangential flow filtration; cation exchange chromatography, e.g. to remove residual protein; and size exclusion chromatography, e.g. to remove low molecular weight contaminants.

The invention also provides a saccharide obtained by the process of the invention.

Thus, the invention provides a composition comprising C. difficile PS-II cell wall saccharide, wherein the composition comprises saccharide and a level of peptidoglycan contamination that is less than 30% (e.g. ≦25%, ≦20%, ≦15%, ≦10%, ≦5%, etc.) by weight peptidoglycan relative to the total weight of the saccharide. Typically, the composition comprises less than 5%, by weight peptidoglycan. The level of peptidoglycan contamination may be measured using the methods described herein, in particular by amino acid analysis using HPAEC-PAD.

Similarly, the invention provides a composition comprising C. difficile PS-II cell wall saccharide, wherein the composition comprises a level of protein contamination that is less than 50% (e.g. ≦40%, ≦30%, ≦20%, ≦10%, etc.) by weight protein relative to the total weight of the saccharide. Typically, the composition comprises less than 5%, by weight protein. The level of protein contamination may be measured using a MicroBCA assay (Pierce). Alternatively, the level of protein contamination may be measured using a Bradford assay.

The invention also provides a composition comprising C. difficile PS-II cell wall saccharide, wherein (a) the level of peptidoglycan contamination is less than 5% (as described above); and (b) the level of protein contamination is less than 5% (as described above).

The invention also provides a process for purifying C. difficile PS-II cell wall saccharide, wherein the process provides a composition comprising saccharide and a level of peptidoglycan contamination that is less than 30% (e.g. ≦25%, ≦20%, ≦15%, ≦10%, ≦5%, etc.) by weight peptidoglycan relative to the total weight of the saccharide. Typically, the composition comprises less than 5%, by weight peptidoglycan. The level of peptidoglycan contamination may be measured using the methods described herein, in particular by amino acid analysis using HPAEC-PAD.

Similarly, the invention provides a process for purifying C. difficile PS-II cell wall saccharide, wherein the process provides a composition comprising a level of protein contamination that is less than 50% (e.g. ≦40%, ≦30%, ≦20%, ≦10%, etc.) by weight protein relative to the total weight of the saccharide.

Typically, the composition comprises less than 5%, by weight protein. The level of protein contamination may be measured using a MicroBCA assay (Pierce). Alternatively, the level of protein contamination may be measured using a Bradford assay.

The invention also provides a process for purifying C. difficile PS-II cell wall saccharide, wherein (a) the level of peptidoglycan contamination is less than 5% (as described above); and (b) the level of protein contamination is less than 5% (as described above).

The invention also provides a saccharide of the invention conjugated to a carrier molecule, such as a protein. In some embodiments, the saccharide is conjugated to the carrier molecule via a linker.

The invention further relates to pharmaceutical compositions comprising a saccharide or conjugate of the invention in combination with a pharmaceutically acceptable carrier.

The invention further relates to methods for raising an immune response in a mammal, comprising administering a saccharide, conjugate or pharmaceutical composition of the invention to the mammal.

### The PS-II Cell Wall Saccharide

The invention relates to the PS-II cell wall saccharide of C. difficile. The structure of the PS-II repeating unit is described in reference 10:

[→6)-D-Glcp-(1→3)-β-D-GalpNAc-(1→4)-α-D-Glcp-(1→4)-β-D-Glcp-(1→3]-β-D-GalpNAc-(1→3)-α-D-Manp-(1→P]

In one aspect, the invention provides a synthetic C. difficile PS-II cell wall saccharide. The saccharide is typically a single molecular species. In an embodiment, the synthetic C. difficile PS-II cell wall saccharide is a hexasaccharide or a dodecasaccharide. The hexasaccharide or dodecasaccharide may lack a phosphate group at the 6-O-position of the non-reducing terminal saccharide of the saccharide. Alternatively, the synthetic C. difficile PS-II cell wall hexasaccharide or dodecasaccharide may comprise a phosphate group at the 6-O-position of the non-reducing terminal saccharide, as in the naturally-occurring saccharide. In a particular embodiment, the saccharide is a hexasaccharide having the following structure (Formula I):

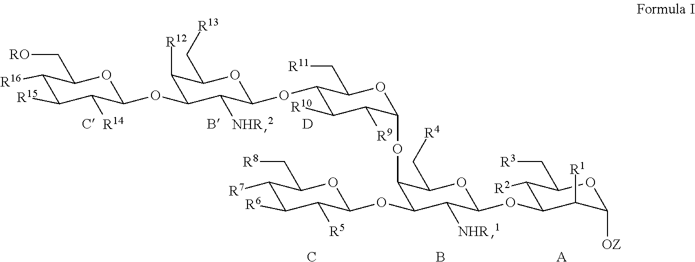

wherein
R is selected from H, PO₃H₂ or an anionic form thereof, acetyl, and a hydroxyl protecting group; each R¹, R², R³, R⁴, R⁵, R⁶, R⁷, R⁸, R⁹, R¹⁰, R¹¹, R¹², R¹³, R¹⁴, R¹⁵ and R¹⁶ is independently selected from OH and a blocking group;
each R,¹ and R,² is independently selected from H, acetyl, and an amino protecting group; and
Z is selected from H, a linker and a hydroxyl protecting group.

Typically, all of R¹, R², R³, R⁴, R⁵, R⁶, R⁷, R⁸, R⁹, R¹⁰, R¹¹, R¹², R¹³, R¹⁴, R¹⁵ and R¹⁶ are OH, as in the naturally-occurring saccharide. However, in some embodiments one or more of these hydroxyl groups are replaced with one or more blocking groups. Blocking groups to replace hydroxyl groups may be directly accessible via a derivatizing reaction of the hydroxyl group i.e. by replacing the hydrogen atom of the hydroxyl group with another group. Suitable derivatives of hydroxyl groups which act as blocking groups are, for example, carbamates, sulfonates, carbonates, esters, ethers (e.g. silyl ethers or alkyl ethers) and acetals. Some specific examples of such blocking groups are allyl, Alloc, benzyl, BOM, t-butyl, trityl, TBS, TBDPS, TES, TMS, TIPS, PMB, MEM, MOM, MTM, THP, etc. Other blocking groups that are not directly accessible and which completely replace the hydroxyl group include C₁₋₁₂ alkyl, C₃₋₁₂ alkyl, C₅₋₁₂ aryl, C₅₋₁₂ aryl-C₁₋₆ alkyl, NR^{a}R^{b} (R^{a} and R^{b} are defined in the following paragraph), H, F, Cl, Br, CO₂H, CO₂(C₁₋₆ alkyl), CN, CF₃, CCl₃, etc.

Typical blocking groups are of the formula: —O-T-Q or —OR^{c} wherein: T is C(O), S(O) or SO₂; Q is C₁₋₁₂ alkyl, C₁₋₁₂ alkoxy, C₃₋₁₂ cycloalkyl, C₅₋₁₂ aryl or C₅₋₁₂ aryl-C₁₋₆ alkyl, each of which may optionally be substituted with 1, 2 or 3 groups independently selected from F, Cl, Br, CO₂H, CO₂(C₁₋₆ alkyl), CN, CF₃ and CCl₃; or Q is NR^{a}R^{b}; R^{a} and R^{b} are independently selected from H, C₁₋₁₂ alkyl, C₃₋₁₂ cycloalkyl, C₅₋₁₂ aryl, C₅₋₁₂ aryl-C₁₋₆ alkyl; or R^{a} and R^{b} may be joined to form a C₃₋₁₂ saturated heterocyclic group; R^{c} is C₁₋₁₂ alkyl or C₃₋₁₂ cycloalkyl, each of which may optionally be substituted with 1, 2 or 3 groups independently selected from F, Cl, Br, CO₂(C₁₋₆ alkyl), CN, CF₃ and CCl₃; or R^{c} is C₅₋₁₂ aryl or C₅₋₁₂ aryl-C₁₋₆ alkyl, each of which may optionally be substituted with 1, 2, 3, 4 or 5 groups selected from F, Cl, Br, CO₂H, CO₂(C₁₋₆ alkyl), CN, CF₃ and CCl₃. When R^{c} is C₁₋₁₂ alkyl or C₃₋₁₂ cycloalkyl, it is typically substituted with 1, 2 or 3 groups as defined above. When R^{a} and R^{b} are joined to form a C₃₋₁₂ saturated heterocyclic group, it is meant that R^{a} and R^{b} together with the nitrogen atom form a saturated heterocyclic group containing any number of carbon atoms between 3 and 12 (e.g. C₃, C₄, C₅, C₆, C₇, C₈, C₉, C₁₀, C₁₁, C₁₂). The heterocyclic group may contain 1 or 2 heteroatoms (such as N, O or S) other than the nitrogen atom. Examples of C₃₋₁₂ saturated heterocyclic groups are pyrrolidinyl, piperidinyl, morpholinyl, piperazinyl, imidazolidinyl, azetidinyl and aziridinyl.

Blocking groups —O-T-Q and —OR^{c} can be prepared from —OH groups by standard derivatizing procedures, such as reaction of the hydroxyl group with an acyl halide, alkyl halide, sulfonyl halide, etc. Hence, the oxygen atom in —O-T-Q is usually the oxygen atom of the hydroxyl group, while the -T-Q group in —O-T-Q usually replaces the hydrogen atom of the hydroxyl group.

Alternatively, the blocking groups may be accessible via a substitution reaction, such as a Mitsonobu-type substitution. These and other methods of preparing blocking groups from hydroxyl groups are well known.

In some embodiments, all of R¹, R², R³, R⁴, R⁵, R⁶, R⁷, R⁸, R⁹, R¹⁰, R¹¹, R¹², R¹³, R¹⁴, R¹⁵ and R¹⁶ are blocking groups. The blocking groups may be the same, or they may be different.

A particularly preferred blocking group is —OC(O)(CH₃).

In a particular embodiment, R¹, R², R³, R⁴, R⁵, R⁶, R⁷, R⁸, R⁹, R¹⁰, R¹¹, R¹², R¹³, R¹⁴, R¹⁵ and R¹⁶ are —OC(O)(CH₃). In another embodiment, R¹, R², R³, R⁴, R⁵, R⁶, R⁷, R⁸, R⁹, R¹⁰, R¹¹, R¹², R¹³, R¹⁴, R¹⁵ and R¹⁶ are OBn.

Typically, R,¹ and R,² are both acetyl, as in the naturally-occurring saccharide. Similarly, R is typically H, PO₃H₂ or an anionic form thereof, or acetyl. Z is typically a linker, which advantageously provides for easy conjugation to a carrier molecule. However, in some embodiments, these groups may be protected hydroxyl or amino groups. This is particularly advantageous when the saccharide is an intermediate used in the preparation of other saccharides, to avoid these groups participating in unwanted reactions. Conventional protecting groups, for example those described in reference 11, may be used to protect such groups.

Hydroxyl groups are typically protected as esters such as methyl, ethyl, benzyl or tert-butyl which can all be removed by hydrolysis in the presence of bases such as lithium or sodium hydroxide. Benzyl (Bn) protecting groups can also be removed by hydrogenation with a palladium catalyst under a hydrogen atmosphere whilst tert-butyl groups can also be removed by trifluoroacetic acid. Alternatively a trichloroethyl ester protecting group is removed with zinc in acetic acid. A common hydroxy protecting group suitable for use herein is a methyl ether. Deprotection conditions comprise refluxing in 48% aqueous HBr for 1-24 hours, or by stirring with borane tribromide in dichloromethane for 1-24 hours. Alternatively where a hydroxyl group is protected as a benzyl ether, deprotection conditions comprise hydrogenation with a palladium catalyst under a hydrogen atmosphere. Other hydroxyl protecting groups include MOM and pivaloyl.

For example, a common amino protecting group suitable for use herein is tert-butoxy carbonyl (Boc), which is readily removed by treatment with an acid such as trifluoroacetic acid or hydrogen chloride in an organic solvent such as dichloromethane. Alternatively the amino protecting group may be a benzyloxycarbonyl group which can be removed by hydrogenation with a palladium catalyst under a hydrogen atmosphere or 9-fluorenylmethyloxycarbonyl (Fmoc) group which can be removed by solutions of secondary organic amines such as diethylamine or piperidine in an organic solvent. Other amino protecting groups include phthalimide, CF₃CO, tetrachlorophthalimide, dimethylmaloyl and 2,2,2-Trichlorethoxycarbonyl chloride (Troc). In a particular embodiment, R,¹ and R,² are both acetyl. In another embodiment, R,¹ and R,² are both Troc.

The invention specifically provides the following embodiments of Formula I:

(1) R is PO₃H₂ or an anionic form thereof all of R¹, R², R³, R⁴, R⁵, R⁶, R⁷, R⁸, R⁹, R¹⁰, R¹¹, R¹², R¹³, R¹⁴, R¹⁵ and R¹⁶ are OH, both R,¹ and R,² are acetyl, and Z is a linker.
(2) R is H, all of R¹, R², R³, R⁴, R⁵, R⁶, R⁷, R⁸, R⁹, R¹⁰, R¹¹, R¹², R¹³, R¹⁴, R¹¹ and R¹⁶ are OH, both R,¹ and R,² are acetyl, and Z is a linker.
(3) R is an acetyl all of R¹, R², R³, R⁴, R⁵, R⁶, R⁷, R⁸, R⁹, R¹⁰, R¹¹, R¹², R¹³, R¹⁴, R¹⁵, and R¹⁶ are OH, both R,¹ and R,² are acetyl, and Z is a linker.
(4) R is PO₃H₂ or an anionic form thereof all of R¹, R², R³, R⁴, R⁵, R⁶, R⁷, R⁸, R⁹, R¹⁰, R¹¹, R¹², R¹³, R¹⁴, R¹⁵ and R¹⁶ are OH, both R,¹ and R,² are acetyl, and Z is H.
(5) R is H, all of R¹, R², R³, R⁴, R⁵, R⁶, R⁷, R⁸, R⁹, R¹⁰, R¹¹, R¹², R¹³, R¹⁴, R¹⁵ and R¹⁶ are OH, both R,¹ and R,² are acetyl, and Z is H.
(6) R is an acetyl, all of R¹, R², R³, R⁴, R⁵, R⁶, R⁷, R⁸, R⁹, R¹⁰, R¹¹, R¹², R¹³, R¹⁴, R¹⁵ and R¹⁶ are OH, both R,¹ and R,² are acetyl, and Z is H.
(7) R is PO₃H₂ or an anionic form thereof all of R¹, R², R³, R⁴, R⁵, R⁶, R⁷, R⁸, R⁹, R¹⁰, R¹¹, R¹², R¹³, R¹⁴, R¹⁵ and R¹⁶ are OH, both R,¹ and R,² are amino protecting groups, and Z is a linker.
(8) R is H, all of R¹, R², R³, R⁴, R⁵, R⁶, R⁷, R⁸, R⁹, R¹⁰, R¹¹, R¹², R¹³, R¹⁴, R¹⁵ and R¹⁶ are OH, both R,¹ and R,² are amino protecting groups, and Z is a linker.
(9) R is an acetyl, all of R¹, R², R³, R⁴, R⁵, R⁶, R⁷, R⁸, R⁹, R¹⁰, R¹¹, R¹², R¹³, R¹⁴, R¹⁵ and R¹⁶ are OH, both R,¹ and R,² are amino protecting groups, and Z is a linker.
(10) R is PO₃H₂ or an anionic form thereof all of R¹, R², R³, R⁴, R⁵, R⁶, R⁷, R⁸, R⁹, R¹⁰, R¹¹, R¹², R¹³, R¹⁴, R¹⁵ and R¹⁶ are OH, both R,¹ and R,² are amino protecting groups, and Z is H.
(11) R is H, all of R¹, R², R³, R⁴, R⁵, R⁶, R⁷, R⁸, R⁹, R¹⁰, R¹¹, R¹², R¹³, R¹⁴, R¹⁵ and R¹⁶ are OH, both R,¹ and R,² are amino protecting groups, and Z is H.
(12) R is an acetyl, all of R¹, R², R³, R⁴, R⁵, R⁶, R⁷, R⁸, R⁹, R¹⁰, R¹¹, R¹², R¹³, R¹⁴, R¹⁵ and R¹⁶ are OH, both R,¹ and R,² are amino protecting groups, and Z is H.

As outlined above, a saccharide of the invention may include a linker. A linker is a covalently attached moiety that facilitates attachment of the saccharide to a carrier molecule. The linker group may be incorporated using any known procedure, for example, the procedures described in references 12 and 13. Typically, the linker is attached via the α-O-position at the reducing terminal saccharide of the PS-II saccharide. A preferred linker is a 1-aminopropyl group. One type of linkage involves reductive amination of the polysaccharide, coupling the resulting amino group with one end of an adipic acid linker group, and then coupling a protein to the other end of the adipic acid linker group [14, 15]. Other linkers include B-propionamido [16], nitrophenyl-ethylamine [17], haloacyl halides [18], glycosidic linkages [19], 6-aminocaproic acid [20], ADH [21], C₄ to C₁₂ moieties [22] etc. As an alternative to using a linker, direct linkage can be used. Direct linkages to the protein may comprise oxidation of the polysaccharide followed by reductive amination with the protein, as described in, for example, references 23 and 24. The linker will generally be added in molar excess to the saccharide during coupling to the saccharide.

The invention also provides intermediates for making the saccharides of the invention. For example, an intermediate specifically envisaged in the present invention is the intermediate of Formula II:

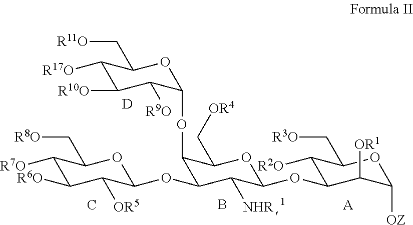

wherein
each R¹, R², R³, R⁴, R⁵, R⁶, R⁷, R⁸, R⁹, R¹⁰, R¹¹, and R¹⁷ is independently selected from OH and a blocking group as defined above;
R,¹ is selected from H, acetyl, and an amino protecting group as defined above; and
Z is selected from H, a linker and a hydroxyl protecting group as defined above.

Typically, all of R¹, R², R³, R⁴, R⁵, R⁶, R⁷, R⁸, R⁹, R¹⁰, R¹¹, and R¹⁷ are OH. Similarly, typically R,¹ is an acetyl. Z is typically H or a linker.

The invention specifically provides the following embodiments of Formula II:

(1) All of R¹, R², R³, R⁴, R⁵, R⁶, R⁷, R⁸, R⁹, R¹⁰, R¹¹, and R¹⁷ are OH, R,¹ is acetyl, and Z is a linker
(2) All of R¹, R², R³, R⁴, R⁵, R⁶, R⁷, R⁸, R⁹, R¹⁰, R¹¹, and R¹⁷ are OH, R,¹ is H, and Z is a linker.
(3) All of R¹, R², R³, R⁴, R⁵, R⁶. R⁷, R⁸, R⁹, R¹⁰, R¹¹, and R¹⁷ are OH, R,¹ is an amino protecting group and Z is a linker.
(4) All of R¹, R², R³, R⁴, R⁵, R⁶, R⁷, R⁸, R⁹, R¹⁰, R¹¹, and R¹⁷ are OH, R,¹ is acetyl, and Z is H.
(5) All of R¹, R², R³, R⁴, R⁵, R⁶, R⁷, R⁸, R⁹, R¹⁰, R¹¹, and R¹⁷ are OH, R,¹ is H, and Z is H.
(6) All of R¹, R², R³, R⁴, R⁵, R⁶, R⁷, R⁸, R⁹, R¹⁰, R¹¹, and R¹⁷ are OH, R,¹ is an amino protecting group and Z is H.

Another intermediate specifically envisaged in the present invention is the intermediate of Formula III:

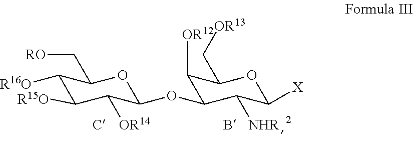

wherein
R is selected from H, PO₃H₂ or an anionic form thereof, acetyl, and a hydroxyl protecting group as defined above;
each R¹², R¹³, R¹⁴, R¹⁵ and R¹⁶ is independently selected from OH and a blocking group as defined above;
R,² is independently selected from H, acetyl, and an amino protecting group as defined above; and
X is an activating group, e.g. selected from OH, SPh, a sulfur protecting group, CNHCCl₃, CF₃CNPh, halogen, O-p-methoxyphenyl, O-pentenyl, OTBS, and OTMS.

Typically, R¹², R¹³, R¹⁴, R¹⁵ and R¹⁶ are OH. Similarly, R,² is typically an acetyl. R is typically H, PO₃H₂ or an anionic form thereof or acetyl. X is typically SPh. However, in some embodiments, X is replaced with a sulfur protecting group. Sulfur protecting groups include methyl, ethyl, phenyl, benzyl, triphenylmethyl, and sulfoxide.

The invention specifically provides the following embodiments of Formula III:

(1) R is PO₃H₂ or an anionic form thereof, all of R¹², R¹³, R¹⁴, R¹⁵ and R¹⁶ are OH, R,² is acetyl, and X is SPh.
(2) R is H, all of R¹², R¹³, R¹⁴, R¹⁵ and R¹⁶ are OH, R,² is acetyl, and X is SPh.
(3) R is PO₃H₂ or an anionic form thereof, all of R¹², R¹³, R¹⁴, R¹⁵ and R¹⁶ are OH, R,² is H, and X is SPh.

### (4) R is H, all of R¹², R¹³, R¹⁴, R¹⁵ and R¹⁶ are OH, R,² is H, and X is SPh.

(5) R is PO₃H₂ or an anionic form thereof, all of R¹², R¹³, R¹⁴, R¹⁵ and R¹⁶ are OH, R,² is an amino protecting group, and X is SPh.
(6) R is H, all of R¹², R¹³, R¹⁴, R¹⁵ and R¹⁶ are OH, R,² is an amino protecting group, and X is SPh.
(7) R is PO₃H₂ or an anionic form thereof, all of R¹², R¹³, R¹⁴, R¹⁵ and R¹⁶ are OH, R,² is acetyl, and X is OH.
(8) R is H, all of R¹², R¹³, R¹⁴, R¹⁵ and R¹⁶ are OH, R,² is acetyl, and X is OH.
(9) R is PO₃H₂ or an anionic form thereof, all of R¹², R¹³, R¹⁴, R¹⁵ and R¹⁶ are OH, R,² is H, and X is OH.

### (10) R is H, all of R¹², R¹³, R¹⁴, R¹⁵ and R¹⁶ are OH, R,² is H, and X is OH.

(11) R is PO₃H₂ or an anionic form thereof, all of R¹², R¹³, R¹⁴, R¹⁵ and R¹⁶ are OH, R,² is an amino protecting group, and X is OH.
(12) R is H, all of R¹², R¹³, R¹⁴, R¹⁵ and R¹⁶ are OH, R,² is an amino protecting group, and X is OH.

The synthetic C. difficile PS-II cell wall saccharide is typically a single molecular species. A saccharide that is a single molecular species may be identified by measuring the polydispersity (Mw/Mn) of the saccharide sample. This parameter can conveniently be measured by SEC-MALLS, for example as described in reference 25. Suitable saccharides of the invention have a polydispersity of about 1, e.g. 1.01 or less.

In an embodiment of the synthetic C. difficile PS-II cell wall saccharide, peptidoglycan contamination is undetectable by HPAEC-PAD and/or protein contamination is undetectable by MicroBCA assay (Pierce). Alternatively, protein contamination may be undetectable by Bradford assay.

The invention also provides a method of making a synthetic C. difficile PS-II cell wall saccharide of the invention. The saccharide may be made in vitro. For example, the saccharide is typically made in glassware, such as a test tube, a round-bottom flask, a volumetric flask or an Erlenmeyer flask. Suitable methods for making the saccharide of the invention include reacting an intermediate according to Formula II with an intermediate according to Formula III. This method may be used to produce a saccharide according to Formula I for example.

The present invention also specifically envisages a PS-II cell wall saccharide, wherein the reducing terminus forms a covalent bond with a linker as in Formula IV:

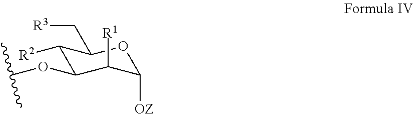

wherein
each R¹, R² and R³ is independently selected from OH and a blocking group; and
Z is a linker.

In comparison to PS-II cell wall saccharide conjugates of the prior art, these saccharides advantageously include the α-configuration at the Cl carbon of the reducing terminus that is found in the naturally occurring saccharide.

The invention specifically provides the following embodiment of Formula IV:

all of R¹, R² and R³ are OH and Z is a linker.

### Purification

In another aspect, the invention provides a process for purifying C. difficile PS-II cell wall saccharide from C. difficile bacterial cells. The bacterial cells are preferably obtained using fermentation. Suitable strains for producing PS-II cell wall saccharide include M68, M120, 630, Nt2023 and Stoke-Mandeville. Other strains may be used. Expression of PS-II by candidate strains may be detected using the method described in section C below. The inventors have found that Stoke-Mandeville is a particularly good producer. After cell growth, the bacterial cells are usually treated using acetic acid, as described below. The saccharide is then purified by processing steps including one or more of fractionation, e.g. to remove protein contaminants; enzymatic treatment, e.g. to remove nucleic acid, protein and/or peptidoglycan contaminants; anion exchange chromatography, e.g. to remove residual protein; concentration using tangential flow filtration; cation exchange chromatography, e.g. to remove residual protein; and size exclusion chromatography, e.g. to remove low molecular weight contaminants. The saccharide may be chemically modified relative to the saccharide as found in nature.

The bacterial cells may be centrifuged prior to release of saccharide. The process may therefore start with the bacterial cells in the form of a wet cell paste. Typically, however, the bacterial cells are resuspended in an aqueous medium that is suitable for the next step in the process, e.g. in a buffer or in distilled water. The bacterial cells may be washed with this medium prior to re-suspension. In another embodiment, the bacterial cells may be treated in suspension in their original culture medium. Alternatively, the bacterial cells are treated in a dried form.

### Acid Treatment

In the process of the invention, C. difficile bacterial cells are treated with acid. This step results in the inactivation of bacterial cells and release of saccharide. In contrast, previous methods have used sodium hypochlorite inactivation, followed by treatment with acid to effect release of saccharide. The inventors have found that using a single step of acid treatment to inactivate the bacterial cells and release the saccharide may reduce contamination. The acid treatment of the invention is preferably carried out using a mild acid, e.g. acetic acid, to minimise damage to the saccharide. The skilled person would be capable of identifying suitable acids and conditions (e.g. of concentration, temperature and/or time) for release of the saccharide. For example, in a typical process, C. difficile bacterial cells are grown for 18-24 hours and are subsequently centrifuged at 12000 g (relative centrifuge force) for 20 minutes. The resulting pellet is washed in phosphate buffered saline solution, suspended in distilled water (3 volumes water:1 volume pellet) and heated to 100° C. in a thermoblock. Acetic acid is added (to a final concentration of 2%) and the solution is kept at 100° C. for two hours, with vortexing every 15 minutes.

Treatment with other acids, e.g. trifluoroacetic or other organic acids, may also be suitable.

After acid treatment, the reaction mixture is typically neutralised. This may be achieved by the addition of a base, e.g. NaOH. The bacterial cells may be centrifuged and the saccharide-containing supernatant collected for storage and/or additional processing. For example, the reaction mixture may be neutralized with an equimolar amount of NaOH and centrifuged at 7000 g (8000 rpm), optionally 6200 g, followed by sterilization with a 0.22 μm pore size filter.

### Fractionation

The saccharide obtained after acid treatment may be impure and contaminated with, for example, bacterial nucleic acids and proteins and thus purification may be needed to obtain useful saccharides. The first stage in the purification process may be fractionation. It is preferred to use a solvent which is relatively selective for the saccharide in order to minimise contaminants (e.g. proteins, nucleic acid etc.). Ethanol has been found to be advantageous in this respect, though other lower alcohols may be used (e.g. methanol, propan-1-ol, propan-2-ol, butan-1-ol, butan-2-ol, 2-methyl-propan-1-ol, 2-methyl-propan-2-ol, diols etc.). The ethanol is preferably added to the precipitated polysaccharide to give a final ethanol concentration (based on total content of ethanol and water) of between 50% and 95% (e.g. around 55%, 60%, 65%, 70%, 75%, 80%, 85%, or around 90%), and preferably between 75% and 95%. The addition of exchanging cations such as calcium or sodium salts facilitates precipitation. Calcium chloride is particularly preferred.

In a typical fractionation process, calcium chloride (e.g. 1%) in a solvent such as ethanol (e.g. 20%) causes precipitation of protein and nucleic acid contaminants, whilst leaving the saccharide in solution. The concentration of ethanol relative to the concentration of calcium chloride is subsequently increased (e.g. from 20% EtOH to 80% EtOH) in order to effect precipitation of the saccharide. This routine may be repeated as necessary throughout the purification process. It is preferred that this routine is repeated after enzymatic treatment. Saccharide is recovered by centrifugation, preferably at 1800 g for 15 minutes.

When present, the fractionation step(s) may be performed after the acid treatment discussed above. Typically, any fractionation step(s) is carried out after the acid treatment discussed above.

### Enzymatic Treatment

The saccharide obtained after acid treatment may be impure and contaminated with bacterial nucleic acids and proteins. This purification may be performed by enzymatic treatment. For example, RNA may be removed by treatment with RNase, DNA with DNase and protein with protease (e.g. pronase). The skilled person would be capable of identifying suitable enzymes and conditions for removal of the contaminants. For example, the inventors have found that treatment of saccharide-containing supernatant (e.g. 10 mM NaPi pH 8.2) with 50 μg/ml each of DNase and RNase at 37° C. for 5-7 or 6-8 hours is suitable. The mixture may then be centrifuged, typically at 1800 g for 15 minutes, optionally 1560 g, and the supernatant adjusted to the desired concentration, e.g. 100 mM NaPi pH 5.9.

The saccharide obtained after acid treatment may also or alternatively be contaminated with peptidoglycan. This contaminant may also be removed by enzymatic treatment. The inventors have found that treatment with mutanolysin is effective at removing peptidoglycan contamination. The skilled person would be capable of identifying suitable conditions for removal of the peptidoglycan with mutanolysin. For example, the inventors have found that treatment of saccharide-containing supernatant with 800 U/ml of mutanolysin at 37° C. for 15-18 hours is suitable. 200 U/ml of mutanolysin at 37° C. for 16 hours has also been found to be suitable. The solution is typically then exposed again to calcium chloride (e.g. 1%) in a solvent such as ethanol (e.g. 20%), followed by an increase in the concentration of ethanol relative to the concentration of calcium chloride (e.g. from 20% EtOH to 80% EtOH) in order to effect precipitation of the saccharide. After treatment, the suspension may be clarified by centrifugation and the saccharide-containing supernatant collected for storage and/or additional processing.

When present, the enzymatic treatment step(s) may be performed after the acid treatment, or fractionation steps discussed above. Typically, any enzymatic treatment step(s) are carried out after the fractionation step discussed above.

### Anion Exchange Chromatography

The saccharide may be further purified by a step of anion exchange chromatography. This step is typically performed after the acid treatment and enzymatic treatment discussed above. This is effective at removing residual protein and nucleic acid contamination, while maintaining a good yield of the saccharide.

Anion exchange chromatography is usually carried out after the acid treatment, fractionation and enzymatic treatment steps described above.

The anion exchange chromatography may be carried out using any suitable anionic exchange matrix. Commonly used anion exchange matrices are resins such as Q-resins (based on quaternary amines). Fractogel-Q® resin (Merck) is particularly suitable, although other resins may be used. Typically, 1 mL of resin is used for 0.2-0.5 mg of PS-II saccharide. The chromatography column is typically equilibrated in 10 mM NaPi buffer at pH 8.0.

Appropriate starting buffers and mobile phase buffers for the anion exchange chromatography can also be determined by routine experiments without undue burden. Typical buffers for use in anion exchange chromatography include N-methyl piperazine, piperazine, L-histidine, bis-Tris, bis-Tris propane, triethanolamine, Tris, N-methyl-diethanolamine, diethanolamine, 1,3-diaminopropane, ethanolamine, piperidine, sodium chloride and phosphate buffers. The inventors have found that phosphate buffers, e.g. a sodium phosphate buffer, are suitable as the starting buffer for the anion exchange chromatography. The buffer may be at any suitable concentration. For example, 10 mM sodium phosphate at pH 8.0 has been found to be suitable. Material bound to the anionic exchange resin may be eluted with a suitable buffer. The inventors have found that a gradient of NaCl 1 M is suitable.

Eluate fractions containing saccharide may be determined by measuring UV absorption at 214 nm. Fractions containing saccharide, usually combined together, are collected for storage and/or additional processing. Fractions may also be analysed for saccharide content using a phenol-sulfuric acid assay [26].

The anion exchange chromatography step may be repeated, e.g. 1, 2, 3, 4 or 5 times. Typically the anion exchange chromatography step is carried out once.

When present, the anion exchange chromatography step(s) may be performed after the acid treatment, fractionation, or enzymatic treatment steps discussed above. Typically, any anion exchange chromatography step(s) are carried out after the enzymatic treatment step discussed above.

### Concentration

The process of the invention may involve one or more steps of concentrating the saccharide. This concentration is useful for obtaining a sample of the correct concentration for any subsequent conjugation of the saccharide to a carrier molecule, as described below.

When present, the concentration step(s) may be performed after the acid treatment, fractionation, enzymatic treatment, or anion exchange chromatography steps discussed above. Typically, any concentration step(s) are carried out after the anion exchange chromatography step(s) discussed above. The concentration step(s) may be repeated, e.g. 1, 2, 3, 4, 5, 6, 7, 8, 9 or 10 times. Typically, any concentration step(s) are repeated 10 times.

The concentration step(s) may be carried out by any suitable method. For example, the inventors have found that the concentration step(s) may be diafiltration step(s), for example tangential flow filtration using a 5 kDa cut-off membrane. For example, a 5 kDa cut-off membrane (with a 200 cm² membrane area) may be used, with a suitable buffer, e.g. 10 mM NaPi buffer at pH 3.0. The filtration membrane should thus be one that allows passage of small molecular weight contaminants while retaining the saccharide. Typically, the inventors use pressure conditions of Pin 1.0 bar, Pout 0.1 bar, and a flow rate of 4 mL/min.

The concentrated saccharide sample is collected for storage and/or additional processing.

### Cation Exchange Chromatography

The saccharide may be further purified by a step of cation exchange chromatography. This is effective at removing positively charged contaminants.

The cation exchange chromatography may be carried out using any suitable cationic exchange matrix. Capto S® resin (G&E healthcare) is particularly suitable, although other resins may be used. Typically, 1 mL of resin is used for 1.0 mg of PS-II saccharide.

Appropriate starting buffers and mobile phase buffers for the cation exchange chromatography can also be determined by routine experiments without undue burden. The inventors have found that phosphate buffers, e.g. a sodium phosphate buffer, are suitable as the starting buffer for the cation exchange chromatography. The buffer may be at any suitable concentration. For example, 10 mM sodium phosphate at pH 3.0 has been found to be suitable. Material bound to the cationic exchange resin may be eluted with a suitable buffer. The inventors have found that a gradient of NaCl 1 M is suitable.

Eluate fractions containing saccharide may be determined by measuring UV absorption at 214 nm. Fractions containing saccharide, usually combined together, are collected for storage and/or additional processing.

The cation exchange chromatography step may be repeated, e.g. 1, 2, 3, 4 or 5 times. Typically the cation exchange chromatography step is carried out once.

When present, the cation exchange chromatography step(s) may be performed after the acid treatment, fractionation, enzymatic treatment, anion exchange chromatography or concentration steps discussed above. Typically, any cation exchange chromatography step(s) are carried out after the concentration step(s) discussed above.

### Size Exclusion Chromatography

The saccharide may be purified using size exclusion chromatography. This is typically carried out using gel-filtration chromatography, for example with Superdex 75 resin. Typically, 1 mL of resin is used for 0.5-0.7 mg of PS-II, and the chromatography column is equilibrated in a suitable buffer, e.g. 10 mM NaPi buffer at pH 7.2.

When present, the size exclusion chromatography step(s) may be performed after the acid treatment, fractionation, enzymatic treatment, anion exchange chromatography, concentration or cation exchange steps discussed above. Typically, any size exclusion chromatography step(s) are carried out after the cation exchange chromatography step(s) discussed above.

### Average Degree of Polymerisation

Fragmentation (e.g. by hydrolysis) of polysaccharides may be performed to give a final average degree of polymerisation (avDP) in the oligosaccharide of less than 20 (e.g. between 5 and 20, preferably around 10). Chemical hydrolysis of saccharides generally involves treatment with either acid or base under conditions that are standard in the art. Conditions for depolymerisation of saccharides are known in the art. One depolymerisation method involves the use of hydrogen peroxide [27]. Hydrogen peroxide is added to a saccharide (e.g. to give a final H₂O₂ concentration of 1%), and the mixture is then incubated (e.g. at around 55° C.) until a desired chain length reduction has been achieved. The reduction over time can be followed by removing samples from the mixture and then measuring the (average) molecular size of saccharide in the sample. Depolymerization can then be stopped by rapid cooling once a desired chain length has been reached.

avDP can conveniently be measured by ion exchange chromatography or by colorimetric assays [28].

### Storage

The C. difficile PS-II cell wall saccharide preparation may be lyophilised, e.g. by freeze-drying under vacuum, or frozen in solution (e.g. as the eluate from the final concentration step, if included) for storage at any stage during the purification process. Accordingly, it is not necessary for the preparation to be transferred immediately from one step of the process to another. For example, if the saccharide preparation is to be purified by diafiltration, then it may be lyophilised or frozen in solution prior to this purification. Similarly, the saccharide may be lyophilised or frozen in solution prior to the anion exchange chromatography step. If the saccharide preparation is to be purified by gel filtration, then it may be lyophilised or frozen in solution prior to this step. Similarly, if the saccharide preparation is to be concentrated, then it may be lyophilised or frozen in solution prior to this step. The lyophilised preparation is reconstituted in an appropriate solution prior to further treatment. Similarly, the frozen solution is defrosted prior to further treatment.

The purified saccharide obtained by the process of the invention may be processed for storage in any suitable way. For example, the saccharide may be lyophilised as described above. Alternatively, the saccharide may be stored in aqueous solution, typically at low temperature, e.g. at −20° C. Conveniently, the saccharide may be stored as the eluate from the anion exchange chromatography, gel filtration or concentration steps.

### Conjugation

The saccharide of the invention, i.e. the synthetic saccharide or a saccharide purified by the above process, can be used as an antigen without further modification e.g. for use in in vitro diagnostic assays, for use in immunisation, etc.

For immunisation purposes, however, it is preferred to conjugate the saccharide to a carrier molecule, such as a protein. In general, covalent conjugation of saccharides to carriers enhances the immunogenicity of saccharides as it converts them from T-independent antigens to T-dependent antigens, thus allowing priming for immunological memory [e.g. ref. 29]. Conjugation is particularly useful for paediatric vaccines [e.g. ref. 30] and is a well known technique [e.g. reviewed in refs. 31 to 39]. Thus the processes of the invention may include the further step of conjugating the purified saccharide to a carrier molecule.

The invention also provides a saccharide of the invention conjugated to a carrier molecule, such as a protein. In some embodiments, saccharide is conjugated to the carrier molecule via a linker. The invention provides a composition comprising: (a) a conjugate of (i) a saccharide of the invention and (ii) a carrier molecule; and optionally (b) an adjuvant.

The carrier molecule may be covalently conjugated to the saccharide directly or via a linker. Any suitable conjugation reaction can be used, with any suitable linker where necessary.

Attachment of the saccharide to the carrier is preferably via a —NH₂ group e.g. in the side chain of a lysine residue in a carrier protein, or of an arginine residue. Attachment to the carrier may also be via a —SH group e.g. in the side chain of a cysteine residue. Alternatively, the saccharide may be attached to the carrier via a linker molecule. The free end of the linker may comprise a group to facilitate conjugation to the carrier protein. For example, the free end of the linker may comprise an amino group.

The linker may be any linker described above.

Preferred carrier proteins are bacterial toxins, such as diphtheria or tetanus toxins, or toxoids or mutants thereof. These are commonly used in conjugate vaccines. The CRM₁₉₇ diphtheria toxin mutant is particularly preferred [40].

Other suitable carrier proteins include the N. meningitidis outer membrane protein complex [41], synthetic peptides [42,43], heat shock proteins [44,45], pertussis proteins [46,47], cytokines [48], lymphokines [48], hormones [48], growth factors [48], human serum albumin (typically recombinant), artificial proteins comprising multiple human CD4⁺ T cell epitopes from various pathogen-derived antigens [49] such as N19 [50], protein D from H. influenzae [51-53], pneumococcal surface protein PspA [54], pneumolysin [55] or its non-toxic derivatives [56], iron-uptake proteins [57], a GBS protein [58], a GAS protein [59] etc.

It is possible to use mixtures of carrier proteins. A single carrier protein may carry multiple different saccharides [60].

Conjugates may have excess carrier (w/w) or excess saccharide (w/w) e.g. polysaccharide:protein ratio (w/w) in the ratio range of 1:20 (i.e. excess protein) to 20:1 (i.e. excess polysaccharide). Ratios of 1:10 to 1:1 are preferred, particularly ratios between 1:5 and 1:2 and, most preferably, about 1:3.

Conjugates may be used in conjunction with free carrier [61]. When a given carrier protein is present in both free and conjugated form in a composition of the invention, the unconjugated form is preferably no more than 5% of the total amount of the carrier protein in the composition as a whole, and more preferably present at less than 2% by weight.

After conjugation, free and conjugated saccharides can be separated. There are many suitable methods, including hydrophobic chromatography, tangential ultrafiltration, diafiltration etc. [refs. 62 & 63, etc.].

The conjugates may be purified using the processes of the invention. In particular, conjugates may be purified using size exclusion chromatography, e.g. with Superdex 75 resin (GE Healthcare).

### Combinations of Saccharides and Other Antigens

Saccharides of the invention (in particular after conjugation as described above) can be mixed e.g. with each other and/or with other antigens. Thus the processes of the invention may include the further step of mixing the saccharide with one or more further antigens. The invention therefore provides a composition comprising a saccharide of the invention and one or more further antigens. The composition is typically an immunogenic composition.

The further antigen(s) may comprise further saccharides of the invention, and so the invention provides a composition comprising more than one saccharide of the invention. Alternatively, the further antigen(s) may be C. difficile saccharides prepared by processes other than those of the invention, e.g. the methods of [10].

The further antigen(s) may comprise other C. difficile antigens, including saccharide and protein antigens.

The further antigen(s) may comprise antigens from non-C. difficile pathogens. Thus the compositions of the invention may further comprise one or more non-C. difficile antigens, including additional bacterial, viral or parasitic antigens. These may be selected from the following:
a protein antigen from N. meningitidis serogroup B, such as those in refs. 64 to 70, with protein ‘287’ (see below) and derivatives (e.g. ‘ΔG287’) being particularly preferred.
an outer-membrane vesicle (OMV) preparation from N. meningitidis serogroup B, such as those disclosed in refs. 71, 72, 73, 74 etc.
a saccharide antigen from N. meningitidis serogroup A, C, W135 and/or Y, such as the oligosaccharide disclosed in ref. 75 from serogroup C or the oligosaccharides of ref. 76.
a saccharide antigen from Streptococcus pneumoniae [e.g. refs. 77-79; chapters 22 & 23 of ref. 86].
an antigen from hepatitis A virus, such as inactivated virus [e.g. 80, 81; chapter 15 of ref. 86].
an antigen from hepatitis B virus, such as the surface and/or core antigens [e.g. 81,82; chapter 16 of ref. 86].
an antigen from hepatitis C virus [e.g. 83].
an antigen from Bordetella pertussis, such as pertussis holotoxin (PT) and filamentous haemagglutinin (FHA) from B. pertussis, optionally also in combination with pertactin and/or agglutinogens 2 and 3 [e.g. refs. 84 & 85; chapter 21 of ref. 86].
a diphtheria antigen, such as a diphtheria toxoid [e.g. chapter 13 of ref. 86].
a tetanus antigen, such as a tetanus toxoid [e.g. chapter 27 of ref. 86].
a saccharide antigen from Haemophilus influenzae B [e.g. chapter 14 of ref. 86]
an antigen from N. gonorrhoeae [e.g. 64, 65, 66].
an antigen from Chlamydia pneumoniae [e.g. 87, 88, 89, 90, 91, 92, 93].
an antigen from Chlamydia trachomatis [e.g. 94].
an antigen from Porphyromonas gingivalis [e.g. 95].
polio antigen(s) [e.g. 96, 97; chapter 24 of ref. 86] such as IPV.
rabies antigen(s) [e.g. 98] such as lyophilised inactivated virus [e.g. 99, RabAvert™].
measles, mumps and/or rubella antigens [e.g. chapters 19, 20 and 26 of ref. 86].
influenza antigen(s) [e.g. chapters 17 & 18 of ref. 86], such as the haemagglutinin and/or neuraminidase surface proteins.
an antigen from Moraxella catarrhalis [e.g. 100].
an antigen from Streptococcus pyogenes (group A streptococcus) [e.g. 101, 102, 103].
an antigen from Streptococcus agalactiae (group B streptococcus) [e.g. 59, 104-106].
an antigen from S. epidermidis [e.g. type I, II and/or III saccharide obtainable from strains ATCC-31432, SE-360 and SE-10 as described in refs. 107, 108 and 109.

Where a saccharide or carbohydrate antigen is used, it is preferably conjugated to a carrier in order to enhance immunogenicity. Conjugation of H. influenzae B, meningococcal and pneumococcal saccharide antigens is well known.

Toxic protein antigens may be detoxified where necessary (e.g. detoxification of pertussis toxin by chemical and/or genetic means [85]).

Where a diphtheria antigen is included in the composition it is preferred also to include tetanus antigen and pertussis antigens. Similarly, where a tetanus antigen is included it is preferred also to include diphtheria and pertussis antigens. Similarly, where a pertussis antigen is included it is preferred also to include diphtheria and tetanus antigens.

Antigens may be adsorbed to an aluminium salt.

One type of preferred composition includes further antigens that affect the immunocompromised, and so the C. difficile saccharides of the invention can be combined with one or more antigens from the following non-C. difficile pathogens: Steptococcus agalactiae, Staphylococcus epidermis, influenza virus, Enterococcus faecalis, Pseudomonas aeruginosa, Legionella pneumophila, Listeria monocytogenes, Neisseria meningitidis, Staphylococcus aureus and parainfluenza virus.

Another type of preferred composition includes further antigens from bacteria associated with nosocomial infections, and so the C. difficile saccharides of the invention can be combined with one or more antigens from the following non-C. difficile pathogens: Staphylococcus aureus, Pseudomonas aeruginosa, Candida albicans, and extraintestinal pathogenic Escherichia coli.

Antigens in the composition will typically be present at a concentration of at least 1 μg/ml each. In general, the concentration of any given antigen will be sufficient to elicit an immune response against that antigen.

As an alternative to using proteins antigens in the composition of the invention, nucleic acid encoding the antigen may be used [e.g. refs. 110 to 118]. Protein components of the compositions of the invention may thus be replaced by nucleic acid (preferably DNA e.g. in the form of a plasmid) that encodes the protein. In practical terms, there may be an upper limit to the number of antigens included in compositions of the invention. The number of antigens (including C. difficile antigens) in a composition of the invention may be less than 20, less than 19, less than 18, less than 17, less than 16, less than 15, less than 14, less than 13, less than 12, less than 11, less than 10, less than 9, less than 8, less than 7, less than 6, less than 5, less than 4, or less than 3. The number of C. difficile antigens in a composition of the invention may be less than 6, less than 5, or less than 4.

### Pharmaceutical Compositions and Methods

The invention provides processes for preparing pharmaceutical compositions, comprising the steps of mixing (a) a saccharide of the invention (optionally in the form of a conjugate) with (b) a pharmaceutically acceptable carrier. Typical ‘pharmaceutically acceptable carriers’ include any carrier that does not itself induce the production of antibodies harmful to the individual receiving the composition. Suitable carriers are typically large, slowly metabolised macromolecules such as proteins, saccharides, polylactic acids, polyglycolic acids, polymeric amino acids, amino acid copolymers, lactose, and lipid aggregates (such as oil droplets or liposomes). Such carriers are well known to those of ordinary skill in the art. The vaccines may also contain diluents, such as water, saline, glycerol, etc. Additionally, auxiliary substances, such as wetting or emulsifying agents, pH buffering substances, and the like, may be present. Sterile pyrogen-free, phosphate-buffered physiologic saline is a typical carrier. A thorough discussion of pharmaceutically acceptable excipients is available in reference 119.

Compositions of the invention may be in aqueous form (i.e. solutions or suspensions) or in a dried form (e.g. lyophilised). If a dried vaccine is used then it will be reconstituted into a liquid medium prior to injection. Lyophilisation of conjugate vaccines is known in the art e.g. the Menjugate™ product is presented in lyophilised form, whereas NeisVac-C™ and Meningitec™ are presented in aqueous form. To stabilise conjugates during lyophilisation, it may be typical to include a sugar alcohol (e.g. mannitol) or a disaccharide (e.g. sucrose or trehalose) e.g. at between 1 mg/ml and 30 mg/ml (e.g. about 25 mg/ml) in the composition.

The pharmaceutical compositions may be packaged into vials or into syringes. The syringes may be supplied with or without needles. A syringe will include a single dose of the composition, whereas a vial may include a single dose or multiple doses.

Aqueous compositions of saccharides of the invention are suitable for reconstituting other vaccines from a lyophilised form. Where a composition of the invention is to be used for such extemporaneous reconstitution, the invention provides a process for reconstituting such a lyophilised vaccine, comprising the step of mixing the lyophilised material with an aqueous composition of the invention. The reconstituted material can be used for injection.

Compositions of the invention may be packaged in unit dose form or in multiple dose form. For multiple dose forms, vials are preferred to pre-filled syringes. Effective dosage volumes can be routinely established, but a typical human dose of the composition has a volume of 0.5 ml e.g. for intramuscular injection.

The pH of the composition is typically between 6 and 8, e.g. about 7. Stable pH may be maintained by the use of a buffer. If a composition comprises an aluminium hydroxide salt, it is typical to use a histidine buffer [120]. The composition may be sterile and/or pyrogen-free. Compositions of the invention may be isotonic with respect to humans.

Compositions of the invention are immunogenic, and are more preferably vaccine compositions. Vaccines according to the invention may either be prophylactic (i.e. to prevent infection) or therapeutic (i.e. to treat infection), but will typically be prophylactic. Immunogenic compositions used as vaccines comprise an immunologically effective amount of antigen(s), as well as any other components, as needed. By ‘immunologically effective amount’, it is meant that the administration of that amount to an individual, either in a single dose or as part of a series, is effective for treatment or prevention. This amount varies depending upon the health and physical condition of the individual to be treated, age, the taxonomic group of individual to be treated (e.g. non-human primate, primate, etc.), the capacity of the individual's immune system to synthesise antibodies, the degree of protection desired, the formulation of the vaccine, the treating doctor's assessment of the medical situation, and other relevant factors. It is expected that the amount will fall in a relatively broad range that can be determined through routine trials.

Within each dose, the quantity of an individual saccharide antigen will generally be between 1-50 g (measured as mass of saccharide) e.g. about 1 μg, about 2.5 μg, about 4 μg, about 5 μg, or about 10 μg.

The compositions of the invention may be prepared in various forms. For example, the compositions may be prepared as injectables, either as liquid solutions or suspensions. The composition may be prepared for pulmonary administration e.g. as an inhaler, using a fine powder or a spray. The composition may be prepared as a suppository or pessary. The composition may be prepared for nasal, aural or ocular administration e.g. as spray, drops, gel or powder [e.g. refs 121 & 122]. Success with nasal administration of pneumococcal saccharides [123,124], Hib saccharides [125], MenC saccharides [126], and mixtures of Hib and MenC saccharide conjugates [127] has been reported.

Compositions of the invention may include an antimicrobial, particularly when packaged in multiple dose format.

Compositions of the invention may comprise detergent e.g. a Tween (polysorbate), such as Tween 80. Detergents are generally present at low levels e.g. <0.01%.

Compositions of the invention may include sodium salts (e.g. sodium chloride) to give tonicity. A concentration of 10±2 mg/ml NaCl is typical.

Compositions of the invention will generally include a buffer. A phosphate buffer is typical.

Compositions of the invention will generally be administered in conjunction with other immunoregulatory agents. In particular, compositions will usually include one or more adjuvants. Such adjuvants include, but are not limited to:

### Mineral-Containing Compositions

Mineral containing compositions suitable for use as adjuvants in the invention include mineral salts, such as aluminium salts and calcium salts. The invention includes mineral salts such as hydroxides (e.g. oxyhydroxides), phosphates (e.g. hydroxyphosphates, orthophosphates), sulphates, etc. [e.g. chapters 8 & 9 of ref. 128], or mixtures of different mineral compounds (e.g. a mixture of a phosphate and a hydroxide adjuvant, optionally with an excess of the phosphate), with the compounds taking any suitable form (e.g. gel, crystalline, amorphous, etc.), and with adsorption to the salt(s) being typical. The mineral containing compositions may also be formulated as a particle of metal salt [129].

Aluminum salts may be included in vaccines of the invention such that the dose of Al³⁺ is between 0.2 and 1.0 mg per dose.

A typical aluminium phosphate adjuvant is amorphous aluminium hydroxyphosphate with PO₄/Al molar ratio between 0.84 and 0.92, included at 0.6 mg Al³⁺/ml. Adsorption with a low dose of aluminium phosphate may be used e.g. between 50 and 100 μg Al³⁺ per conjugate per dose. Where an aluminium phosphate it used and it is desired not to adsorb an antigen to the adjuvant, this is favoured by including free phosphate ions in solution (e.g. by the use of a phosphate buffer).

### Oil Emulsions

Oil emulsion compositions suitable for use as adjuvants in the invention include squalene-water emulsions, such as MF59 (5% Squalene, 0.5% Tween 80, and 0.5% Span 85, formulated into submicron particles using a microfluidizer) [Chapter 10 of ref. 128; also refs. 130-132]. MF59 is used as the adjuvant in the FLUAD™ influenza virus trivalent subunit vaccine.

Particularly useful adjuvants for use in the compositions are submicron oil-in-water emulsions. Preferred submicron oil-in-water emulsions for use herein are squalene/water emulsions optionally containing varying amounts of MTP-PE, such as a submicron oil-in-water emulsion containing 4-5% w/v squalene, 0.25-1.0% w/v Tween 80 (polyoxyelthylenesorbitan monooleate), and/or 0.25-1.0% Span 85 (sorbitan trioleate), and, optionally, N-acetylmuramyl-L-alanyl-D-isogluatminyl-L-alanine-2-(1′-2′-dipalmitoyl-sn-glycero-3-hydroxyphosphosphoryloxy)-ethylamine (MTP-PE). Submicron oil-in-water emulsions, methods of making the same and immunostimulating agents, such as muramyl peptides, for use in the compositions, are described in detail in references 130 & 133-134.

Complete Freund's adjuvant (CFA) and incomplete Freund's adjuvant (IFA) may also be used as adjuvants in the invention.

### Saponin Formulations [Chapter 22 of Ref 128]

Saponin formulations may also be used as adjuvants in the invention. Saponins are a heterologous group of sterol glycosides and triterpenoid glycosides that are found in the bark, leaves, stems, roots and even flowers of a wide range of plant species. Saponins isolated from the bark of the Quillaia saponaria Molina tree have been widely studied as adjuvants. Saponin can also be commercially obtained from Smilax ornata (sarsaprilla), Gypsophilla paniculata (brides veil), and Saponaria officianalis (soap root). Saponin adjuvant formulations include purified formulations, such as QS21, as well as lipid formulations, such as ISCOMs.

Saponin compositions have been purified using HPLC and RP-HPLC. Specific purified fractions using these techniques have been identified, including QS7, QS17, QS18, QS21, QH-A, QH-B and QH-C.

Preferably, the saponin is QS21. A method of production of QS21 is disclosed in ref. 135. Saponin formulations may also comprise a sterol, such as cholesterol [136].

Combinations of saponins and cholesterols can be used to form unique particles called immunostimulating complexes (ISCOMs) [chapter 23 of ref. 128]. ISCOMs typically also include a phospholipid such as phosphatidylethanolamine or phosphatidylcholine. Any known saponin can be used in ISCOMs. Preferably, the ISCOM includes one or more of QuilA, QHA and QHC. ISCOMs are further described in refs. 136-138. Optionally, the ISCOMS may be devoid of additional detergent(s) [139].

A review of the development of saponin based adjuvants can be found in refs. 140 & 141.

### Virosomes and Virus-Like Particles

Virosomes and virus-like particles (VLPs) can also be used as adjuvants in the invention. These structures generally contain one or more proteins from a virus optionally combined or formulated with a phospholipid. They are generally non-pathogenic, non-replicating and generally do not contain any of the native viral genome. The viral proteins may be recombinantly produced or isolated from whole viruses. These viral proteins suitable for use in virosomes or VLPs include proteins derived from influenza virus (such as HA or NA), Hepatitis B virus (such as core or capsid proteins), Hepatitis E virus, measles virus, Sindbis virus, Rotavirus, Foot-and-Mouth Disease virus, Retrovirus, Norwalk virus, human Papilloma virus, HIV, RNA-phages, Qβ-phage (such as coat proteins), GA-phage, fr-phage, AP205 phage, and Ty (such as retrotransposon Ty protein p1). VLPs are discussed further in refs. 142-147. Virosomes are discussed further in, for example, ref. 148.

### Bacterial or Microbial Derivatives

Adjuvants suitable for use in the invention include bacterial or microbial derivatives such as non-toxic derivatives of enterobacterial liposaccharide (LPS), Lipid A derivatives, immunostimulatory oligonucleotides and ADP-ribosylating toxins and detoxified derivatives thereof.

Non-toxic derivatives of LPS include monophosphoryl lipid A (MPL) and 3-O-deacylated MPL (3dMPL). 3dMPL is a mixture of 3 de-O-acylated monophosphoryl lipid A with 4, 5 or 6 acylated chains. A preferred “small particle” form of 3 De-O-acylated monophosphoryl lipid A is disclosed in ref. 149. Such “small particles” of 3dMPL are small enough to be sterile filtered through a 0.22 μm membrane [149]. Other non-toxic LPS derivatives include monophosphoryl lipid A mimics, such as aminoalkyl glucosaminide phosphate derivatives e.g. RC-529 [150,151].

Lipid A derivatives include derivatives of lipid A from Escherichia coli such as OM-174. OM-174 is described for example in refs. 152 & 153.

Immunostimulatory oligonucleotides suitable for use as adjuvants in the invention include nucleotide sequences containing a CpG motif (a dinucleotide sequence containing an unmethylated cytosine linked by a phosphate bond to a guanosine). Double-stranded RNAs and oligonucleotides containing palindromic or poly(dG) sequences have also been shown to be immunostimulatory.

The CpG's can include nucleotide modifications/analogs such as phosphorothioate modifications and can be double-stranded or single-stranded. References 154, 155 and 156 disclose possible analog substitutions e.g. replacement of guanosine with 2′-deoxy-7-deazaguanosine. The adjuvant effect of CpG oligonucleotides is further discussed in refs. 157-162.

The CpG sequence may be directed to TLR9, such as the motif GTCGTT or TTCGTT [163]. The CpG sequence may be specific for inducing a Th1 immune response, such as a CpG-A ODN, or it may be more specific for inducing a B cell response, such a CpG-B ODN. CpG-A and CpG-B ODNs are discussed in refs. 164-166. Preferably, the CpG is a CpG-A ODN.

Preferably, the CpG oligonucleotide is constructed so that the 5′ end is accessible for receptor recognition. Optionally, two CpG oligonucleotide sequences may be attached at their 3′ ends to form “immunomers” (e.g. refs. 163 & 167-169).

Bacterial ADP-ribosylating toxins and detoxified derivatives thereof may be used as adjuvants in the invention. Preferably, the protein is derived from E. coli (E. coli heat labile enterotoxin “LT”), cholera (“CT”), or pertussis (“PT”). The use of detoxified ADP-ribosylating toxins as mucosal adjuvants is described in ref. 170 and as parenteral adjuvants in ref. 171. The toxin or toxoid is preferably in the form of a holotoxin, comprising both A and B subunits. Preferably, the A subunit contains a detoxifying mutation; preferably the B subunit is not mutated. Preferably, the adjuvant is a detoxified LT mutant such as LT-K63, LT-R72, and LT-G192. The use of ADP-ribosylating toxins and detoxified derivaties thereof, particularly LT-K63 and LT-R72, as adjuvants can be found in refs. 172-179. Numerical reference for amino acid substitutions is preferably based on the alignments of the A and B subunits of ADP-ribosylating toxins set forth in ref. 180, specifically incorporated herein by reference in its entirety.

### Human Immunomodulators

Human immunomodulators suitable for use as adjuvants in the invention include cytokines, such as interleukins (e.g. IL-1, IL-2, IL-4, IL-5, IL-6, IL-7, IL-12 [181], etc.) [182], interferons (e.g. interferon-γ), macrophage colony stimulating factor, and tumor necrosis factor.

### Bioadhesives and Mucoadhesives

Bioadhesives and mucoadhesives may also be used as adjuvants in the invention. Suitable bioadhesives include esterified hyaluronic acid microspheres [183] or mucoadhesives such as cross-linked derivatives of poly(acrylic acid), polyvinyl alcohol, polyvinyl pyrollidone, polysaccharides and carboxymethylcellulose. Chitosan and derivatives thereof may also be used as adjuvants in the invention [184].

### Microparticles

Microparticles may also be used as adjuvants in the invention. Microparticles (i.e. a particle of ˜100 nm to ˜150 μm in diameter, more preferably ˜200 nm to ˜30 μm in diameter, and most preferably ˜500 nm to ˜10 μm in diameter) formed from materials that are biodegradable and non-toxic (e.g. a poly(α-hydroxy acid), a polyhydroxybutyric acid, a polyorthoester, a polyanhydride, a polycaprolactone, etc.), with poly(lactide-co-glycolide) are preferred, optionally treated to have a negatively-charged surface (e.g. with SDS) or a positively-charged surface (e.g. with a cationic detergent, such as CTAB).

### Liposomes (Chapters 13 & 14 of Ref 128)

Examples of liposome formulations suitable for use as adjuvants are described in refs. 185-187.

### Polyoxyethylene Ether and Polyoxyethylene Ester Formulations

Adjuvants suitable for use in the invention include polyoxyethylene ethers and polyoxyethylene esters [188]. Such formulations further include polyoxyethylene sorbitan ester surfactants in combination with an octoxynol [189] as well as polyoxyethylene alkyl ethers or ester surfactants in combination with at least one additional non-ionic surfactant such as an octoxynol [190]. Preferred polyoxyethylene ethers are selected from the following group: polyoxyethylene-9-lauryl ether (laureth 9), polyoxyethylene-9-steoryl ether, polyoxytheylene-8-steoryl ether, polyoxyethylene-4-lauryl ether, polyoxyethylene-35-lauryl ether, and polyoxyethylene-23-lauryl ether.

### Polyphosphazene (PCPP)

PCPP formulations are described, for example, in refs. 191 and 192.

### Muramyl Peptides

Examples of muramyl peptides suitable for use as adjuvants in the invention include N-acetyl-muramyl-L-threonyl-D-isoglutamine (thr-MDP), N-acetyl-normuramyl-L-alanyl-D-isoglutamine (nor-MDP), and N-acetylmuramyl-L-alanyl-D-isoglutaminyl-L-alanine-2-(1′-2′-dipalmitoyl-sn-glycero-3-hydroxyphosphoryloxy)-ethylamine MTP-PE).

### Imidazoquinolone Compounds.

Examples of imidazoquinolone compounds suitable for use adjuvants in the invention include Imiquamod and its homologues (e.g. “Resiquimod 3M”), described further in refs. 193 and 194.

### Thiosemicarbazone Compounds.

Examples of thiosemicarbazone compounds, as well as methods of formulating, manufacturing, and screening for compounds all suitable for use as adjuvants in the invention include those described in ref. 195. The thiosemicarbazones are particularly effective in the stimulation of human peripheral blood mononuclear cells for the production of cytokines, such as TNF-α.

### Tryptanthrin Compounds.

Examples of tryptanthrin compounds, as well as methods of formulating, manufacturing, and screening for compounds all suitable for use as adjuvants in the invention include those described in ref. 196. The tryptanthrin compounds are particularly effective in the stimulation of human peripheral blood mononuclear cells for the production of cytokines, such as TNF-α.

The invention may also comprise combinations of aspects of one or more of the adjuvants identified above. For example, the following combinations may be used as adjuvant compositions in the invention: (1) a saponin and an oil-in-water emulsion [197]; (2) a saponin (e.g. QS21)+a non-toxic LPS derivative (e.g. 3dMPL) [198]; (3) a saponin (e.g. QS21)+a non-toxic LPS derivative (e.g. 3dMPL)+a cholesterol; (4) a saponin (e.g. QS21)+3dMPL+IL-12 (optionally+a sterol) [199]; (5) combinations of 3dMPL with, for example, QS21 and/or oil-in-water emulsions [200]; (6) SAF, containing 10% squalane, 0.4% Tween 80™, 5% pluronic-block polymer L121, and thr-MDP, either microfluidized into a submicron emulsion or vortexed to generate a larger particle size emulsion. (7) Ribi™ adjuvant system (RAS), (Ribi Immunochem) containing 2% squalene, 0.2% Tween 80, and one or more bacterial cell wall components from the group consisting of monophosphorylipid A (MPL), trehalose dimycolate (TDM), and cell wall skeleton (CWS), preferably MPL+CWS (Detox™); and (8) one or more mineral salts (such as an aluminum salt)+a non-toxic derivative of LPS (such as 3dMPL).

Other substances that act as immunostimulating agents are disclosed in chapter 7 of ref. 128.

The use of aluminium salt adjuvants is particularly useful, and antigens are generally adsorbed to such salts. The Menjugate™ and NeisVac™ conjugates use a hydroxide adjuvant, whereas Meningitec™ uses a phosphate adjuvant. It is possible in compositions of the invention to adsorb some antigens to an aluminium hydroxide but to have other antigens in association with an aluminium phosphate. Typically, however, only a single salt is used, e.g. a hydroxide or a phosphate, but not both. Not all conjugates need to be adsorbed i.e. some or all can be free in solution.

### Methods of Treatment

The invention also provides a method for raising an immune response in a mammal, comprising administering a pharmaceutical composition of the invention to the mammal. The immune response is preferably protective and preferably involves antibodies. The method may raise a booster response.

The mammal is preferably a human. Where the vaccine is for prophylactic use, the human is preferably a child (e.g. a toddler or infant) or a teenager; where the vaccine is for therapeutic use, the human is preferably an adult. A vaccine intended for children may also be administered to adults e.g. to assess safety, dosage, immunogenicity, etc. A preferred class of humans for treatment are patients at risk of nosocomial infection, particularly those with end-stage renal disease and/or on haemodialysis. Other patients at risk of nosocomial infection are also preferred, e.g. immunodeficient patients or those who have undergone surgery, especially cardiac surgery, or trauma. Another preferred class of humans for treatment are patients at risk of bacteremia.

The invention also provides a composition of the invention for use as a medicament. The medicament is preferably able to raise an immune response in a mammal (i.e. it is an immunogenic composition) and is more preferably a vaccine.

The invention also provides the use of a conjugate of the invention in the manufacture of a medicament for raising an immune response in a mammal.

These uses and methods are preferably for the prevention and/or treatment of a disease caused by C. difficile, e.g. diarrhea, colitis, peritonitis, septicaemia and perforation of the colon.

One way of checking efficacy of therapeutic treatment involves monitoring S. aureus infection after administration of the composition of the invention. One way of checking efficacy of prophylactic treatment involves monitoring immune responses against the S. aureus antigens after administration of the composition.

Preferred compositions of the invention can confer an antibody titre in a patient that is superior to the criterion for seroprotection for each antigenic component for an acceptable percentage of human subjects. Antigens with an associated antibody titre above which a host is considered to be seroconverted against the antigen are well known, and such titres are published by organisations such as WHO. Preferably more than 80% of a statistically significant sample of subjects is seroconverted, more preferably more than 90%, still more preferably more than 93% and most preferably 96-100%.

Compositions of the invention will generally be administered directly to a patient. Direct delivery may be accomplished by parenteral injection (e.g. subcutaneously, intraperitoneally, intravenously, intramuscularly, or to the interstitial space of a tissue), or by rectal, oral, vaginal, topical, transdermal, intranasal, ocular, aural, pulmonary or other mucosal administration. Intramuscular administration to the thigh or the upper arm is preferred. Injection may be via a needle (e.g. a hypodermic needle), but needle-free injection may alternatively be used. A typical intramuscular dose is 0.5 ml.

The invention may be used to elicit systemic and/or mucosal immunity.

Dosage treatment can be a single dose schedule or a multiple dose schedule. Multiple doses may be used in a primary immunisation schedule and/or in a booster immunisation schedule. A primary dose schedule may be followed by a booster dose schedule. Suitable timing between priming doses (e.g. between 4-16 weeks), and between priming and boosting, can be routinely determined.

### General

The practice of the present invention will employ, unless otherwise indicated, conventional methods of chemistry, biochemistry, molecular biology, immunology and pharmacology, within the skill of the art. Such techniques are explained fully in the literature (e.g., refs. 201-208, etc.).

The term “comprising” encompasses “including” as well as “consisting” e.g. a composition “comprising” X may consist exclusively of X or may include something additional e.g. X+Y.

The term “about” in relation to a numerical value x means, for example, x+10%.

The word “substantially” does not exclude “completely” e.g. a composition which is “substantially free” from Y may be completely free from Y. Where necessary, the word “substantially” may be omitted from the definition of the invention.

Where the invention provides a process involving multiple sequential steps, the invention can also provide a process involving less than the total number of steps. The different steps can be performed at very different times by different people in different places (e.g. in different countries).

It will be appreciated that sugar rings can exist in open and closed form and that, whilst closed forms are shown in structural formulae herein, open forms are also encompassed by the invention. Similarly, it will be appreciated that sugars can exist in pyranose and furanose forms and that, whilst pyranose forms are shown in structural formulae herein, furanose forms are also encompassed. Different anomeric forms of sugars are also encompassed.

### BRIEF DESCRIPTION OF DRAWINGS

FIG. 1a shows the structure of a synthetic tetrasaccharide conjugated to a carrier protein through SIDEA activation. FIG. 1b shows an SDS-PAGE analysis of the tetrasaccharide-carrier protein conjugate.

FIG. 2 shows a Superdex 75 chromatogram of the tetrasaccharide-carrier protein conjugate of FIG. 1.

FIG. 3a shows the structure of a synthetic non-phosphorylated PS-II cell wall hexasaccharide conjugated to a carrier protein through SIDEA activation. FIG. 3b shows an SDS-PAGE analysis of the hexasaccharide-carrier protein conjugate.

FIG. 4 shows the structure of a synthetic phosphorylated PS-II cell wall hexasaccharide conjugated to a carrier protein through SIDEA activation.

FIG. 5a shows an SDS-PAGE analysis of two synthetic non-phosphorylated PS-II cell wall hexasaccharide-protein conjugates (Hexa1-CRM₁₉₇ (4) and Hexa1a-CRM₁₉₇ (5)), one synthetic phosphorylated PS-II cell wall hexasaccharide-protein conjugate (Hexa2-CRM₁₉₇ (6)) and two non-phosphorylated PS-II tetrasaccharide-carrier protein conjugates (Tetra1-CRM₁₉₇ (2) and Tetra1a-CRM₁₉₇ (3)). CRM₁₉₇ is shown at position (1). FIG. 5b shows the results of MALDI-TOF spectrometry on these saccharide conjugates.

FIG. 6 shows the structure of the C. difficile cell-surface saccharide (PS-II), which is composed of hexaglycosyl phosphate repeating units.

FIG. 7 compares the results of High Performance Anion Exchange Chromatography with Pulsed Amperometric Detection (HPAEC-PAD) analysis of C. difficile PS-II of Monteiro et al. with two pools of C. difficile PS-II of the present invention (i.e. by means of a Superdex 75 chromatogram).

FIG. 8 shows the steps carried out to effect conjugation of C. difficile PS-II to CRM₁₉₇.

FIG. 9a shows a Superdex 75 chromatogram of the PS-II-CRM₁₉₇ conjugate of FIG. 8. FIG. 9b shows an SDS-PAGE analysis of the PS-II-CRM₁₉₇ conjugate made using a method of the invention.

FIG. 10 shows the results of High Performance Anion Exchange Chromatography with Pulsed Amperometric Detection (HPAEC-PAD) analysis of PS-II-CRM₁₉₇ total saccharide and SPE & HPAEC-PAD analysis of free saccharide.

FIG. 11a compares the IgG response to different conjugates after three doses using mice sera based on direct coating of PS-II on the plates. FIG. 11b compares IgG and IgM responses to various conjugates after three doses using mice sera based on direct coating of PS-1 on the plates.

FIG. 12 compares the IgG response to different conjugates after three doses using mice sera based on plates coated with PS-II-CRM₁₉₇ conjugate.

FIG. 13 shows the results of competitive ELISA studies carried out using sera of mice immunized with PS-II-CRM₁₉₇ conjugate against PS-II conjugated to recombinant protein from C. difficile.

FIG. 14a compares the IgG response to different conjugates after three doses using mice sera based on plates coated with PS-II-HSA, with AlumOH as adjuvant. FIG. 14b compares the IgG response to different conjugates after three doses using mice sera based on plates coated with PS-II-HSA, with MF59 as adjuvant. The references in FIG. 14a, in which Alum is adjuvant, denote the following: (A) PBS+Alum; (B) Tetra1-CRM₁₉₇ (non-phosphorylated); (C) Hexa1a-CRM₁₉₇ (non-phosphorylated); and (D) Hexa2-CRM₁₉₇ (phosphorylated). The references in FIG. 14b, in which MF59 is adjuvant, denote the following: (E) PBS+MF59; (F) Tetra1-CRM₁₉₇ (non-phosphorylated); (G) Hexa1a-CRM₁₉₇ (non-phosphorylated); (H) Hexa2-CRM₁₉₇ (phosphorylated); and (1) Purified native PS-II-CRM₁₉₇.

FIG. 15 shows the IgG response to PS-II-CRM₁₉₇ using sera from BALB/c mice on plates coated with PS-II-HSA.

### MODES FOR CARRYING OUT THE INVENTION

### A. Synthesis of C. Difficile PS-II Saccharides

The inventors have carried out the first synthesis of the hexasaccharide PS-II repeating unit 2 and its non-phosphorylated analogue 1. A retrosynthetic analysis is shown in scheme 1.

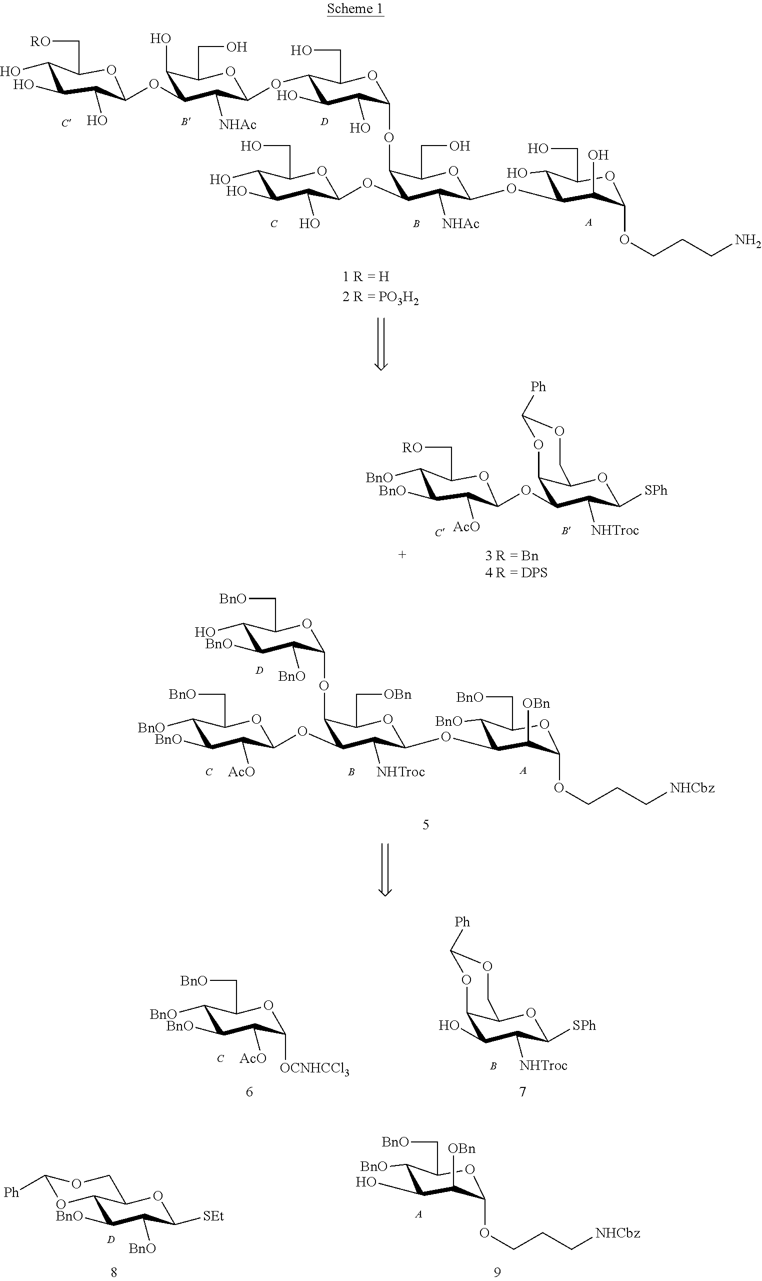

Both oligosaccharides were synthesized with an O-linked aminopropyl spacer at the reducing end suitable for conjugation to a carrier protein, which is a fundamental step to make poorly immunogenic carbohydrates able to induce a T cell dependent response [209]. According to our retrosynthetic analysis (scheme 1), target hexasaccharides 1 and 2 could be assembled by via a tetrasaccharide intermediate 5 (scheme 1). This strategy features disaccharide 3 as a key intermediate both for the synthesis of tetrasaccharide 5 and the construction of hexasaccharide 1. In addition, the challenging insertion of the 1,2-cis glycosidic linkage between residues 7 and 8 should be carried out in an early stage of the synthesis.

On the other hand, the preparation of the phosphorylated hexasaccharide 2 required disaccharide donor 4, which differs from 3 by a further selectively removable group at the primary hydroxyl of the C′ unit.

The inventors employed the N-trichloroethoxycarbonyl (Troc) participating group for the amino group protection in the galactosamine units of 3 and 4 (references 210 and 211), to ensure the formation of 1,2-trans glycosidic linkages.

Another strategy for synthesising saccharides 1 and 2 is shown in scheme 2. The inventors have found that better yields were achieved using the strategy described in scheme 1.

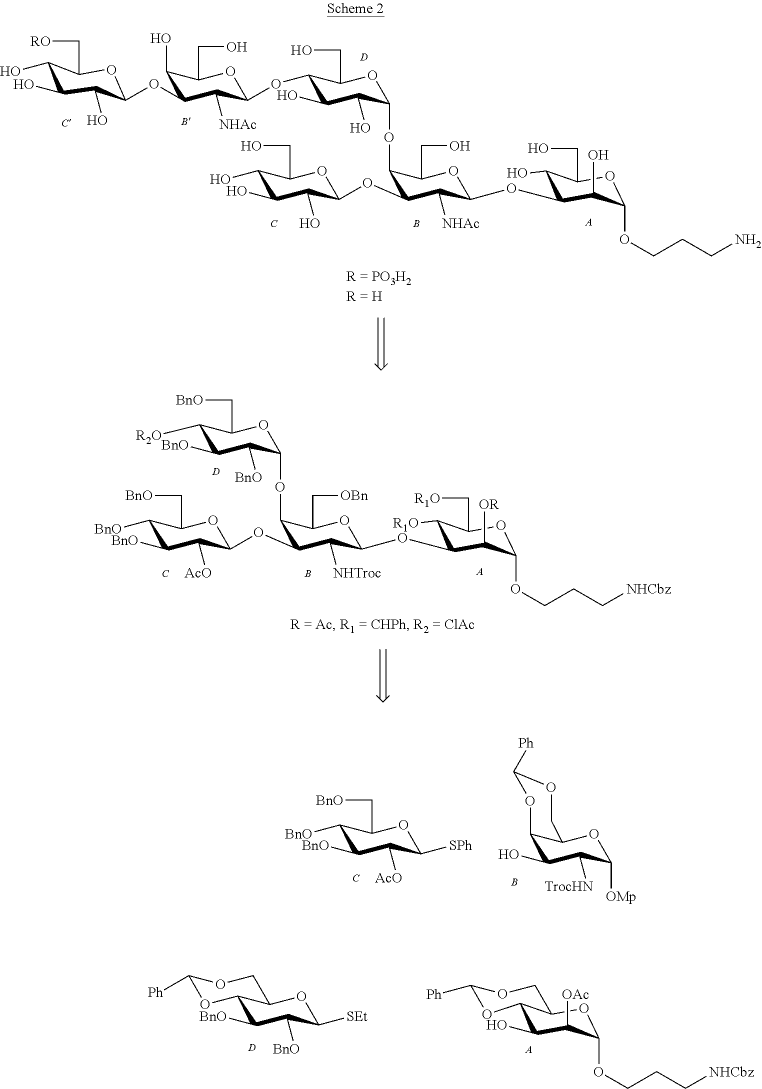

The tetrasaccharide intermediate shown in scheme 2 was deprotected to provide the corresponding tetrasaccharide fragment of the PS-II repeating unit, as shown in scheme 3. This was subsequently conjugated to carrier protein CRM₁₉₇ in order to enable information to be gathered regarding the immunogenicity of the tetrasaccharide repeating unit, i.e. where the disaccharide unit Glc-GalNAc is absent.

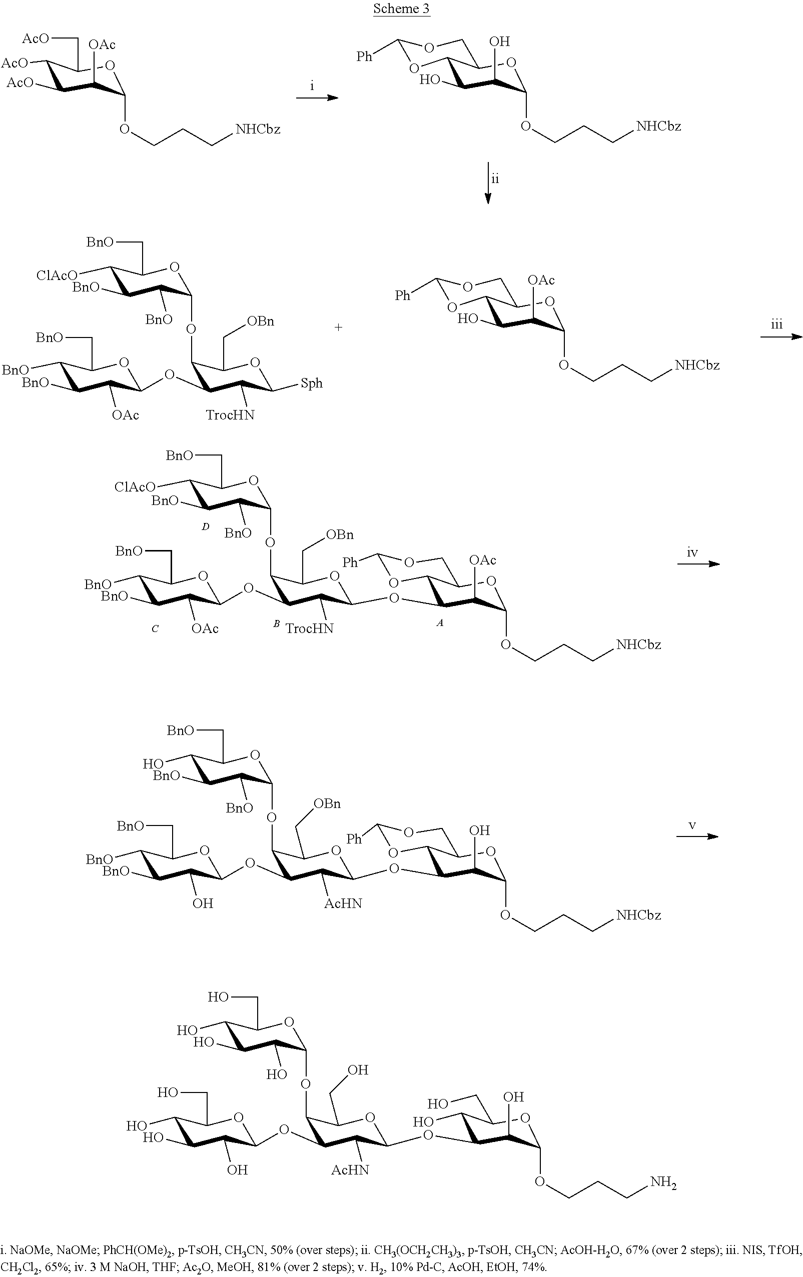

The synthesis of 2,4,6-tri-O-benzylated mannoside 9, bearing the α-oriented anomeric linker, was carried out as illustrated in Scheme 4.

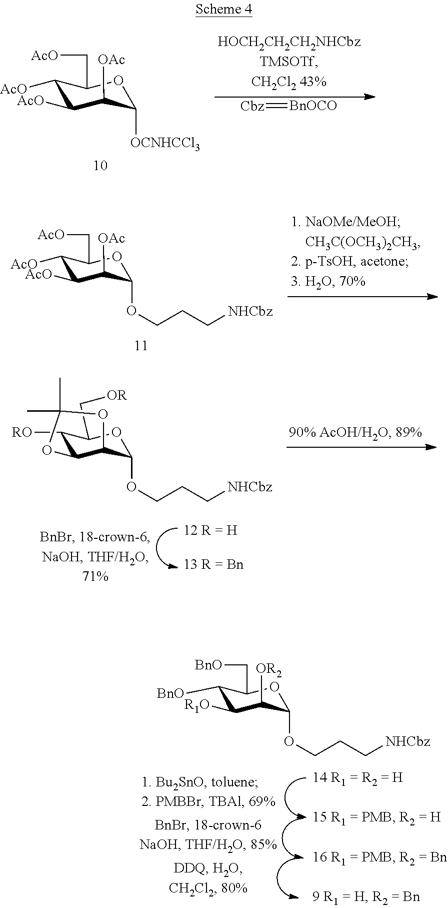

After glycosylation of commercially available benzyl N-(3-hydroxypropyl)carbamate with donor 10 (reference 212), the resulting mannoside 11 was deacetylated and converted into the 2,3-O-isopropylidene derivative 12 by 2,3:4,6-bis isopropylidenation followed by selective monohydrolysis (reference 213). Benzylation of diol 12, using NaOH as a base under phase transfer conditions in order to prevent concomitant N-benzylation of the linker (reference 212), and subsequent isopropylidene acetal hydrolysis afforded compound 14. Regioselective p-methoxybenzylation at 3-OH of 14 using the stannylene acetal protocol allowed the benzylation of the axial 2-hydroxyl (reference 214). Finally, standard oxidation of 16 with 3-dichloro-5,6-dicyano-1,4-benzoquinone provided the 3-OH mannosyl acceptor 9.

The syntheses of disaccharide donors 3 and 4 were achieved starting from the N-Troc galactosamine acceptor 7 (Scheme 5), obtained from the known compound 17 (reference 215) by deacetylation and introduction of the 4,6-O-benzylidene acetal.

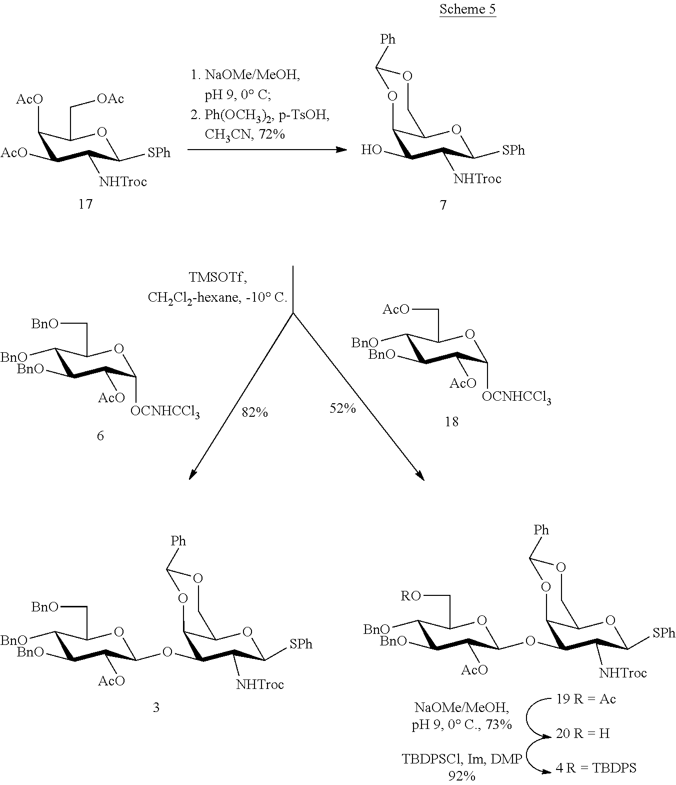

Glycosylation of monosaccharide acceptor 7 with trichloroacedimidate donor 6 (reference 216) in the presence of TMSOTf as a Lewis acid required dichloromethane-hexane as a solvent mixture to circumvent formation of the 1-N-trichloroacetamide (reference 217), which was the predominant by-product when the reaction was performed in only dichloromethane. Disaccharide 3 could be obtained in a satisfactory 82% yield, whereas the preparation of the closely related compound 19 from donor 18 (reference 218 proceeded in lower yield (52%). Disaccharide 19 was then regioselectively 6-O-deacetylated by mild transesterification with NaOMe at pH 9 and 0° C., allowing the straightforward introduction of the t-butyldiphenylsilyl protecting group to afford compound 4.

Thioglycoside 3 was used as a donor for glycosylation of the acceptor 9 promoted by NIS-TfOH, giving trisaccharide 21 in 77% yield (Scheme 6).

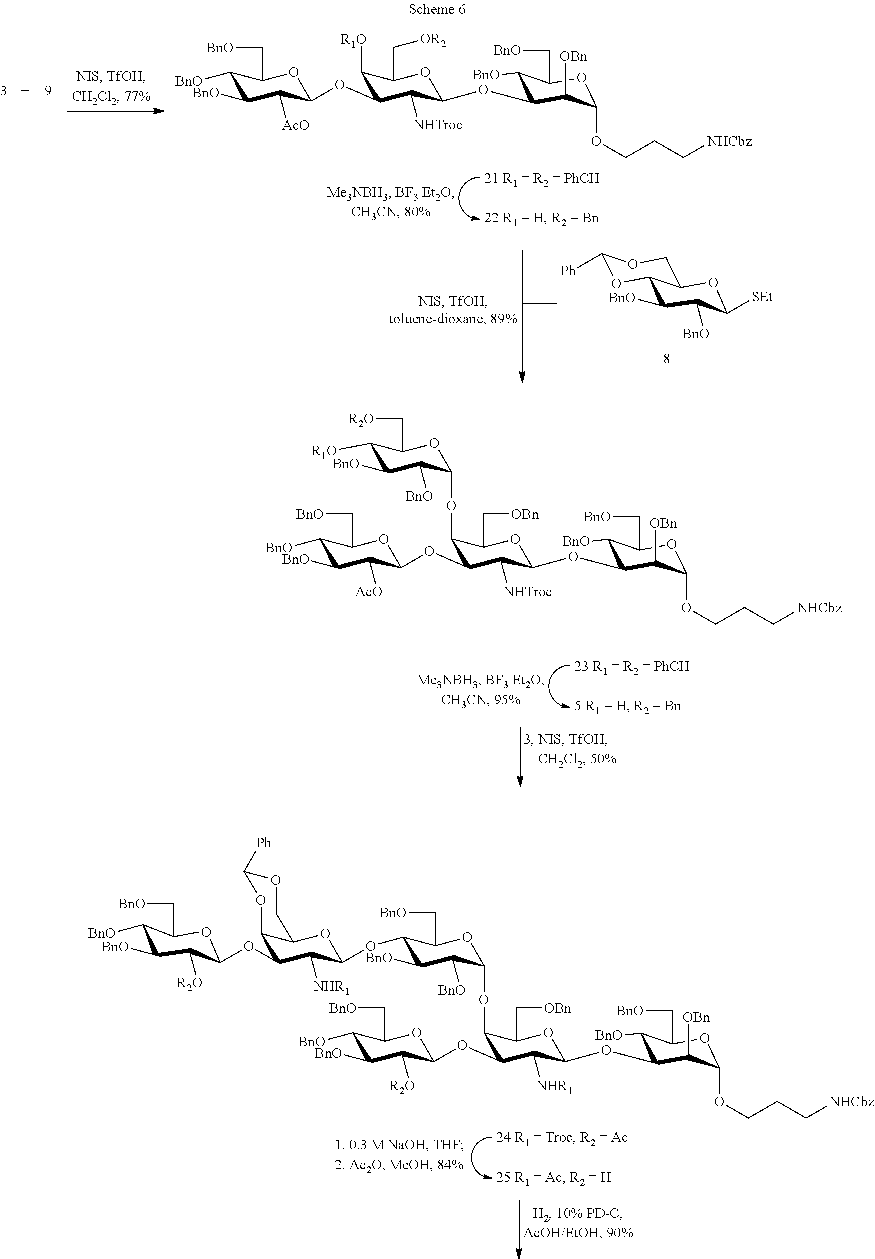

Compound 21 was subjected to regioselective opening of the benzylidene acetal by borane-trimethylamine complex and BF₃.Et₂O (reference 219 and 220) to directly furnish the trisaccharide acceptor 22 (80% yield). Gratifyingly, the glycosylation of 22 with ethylthioglycoside 8 (reference 221) in toluene-dioxane using NIS-TfOH as promoters permitted the stereoselective introduction of the α-linkage and provided tetrasaccharide 23 in 89% yield. The α configuration of the newly formed glycosidic bond was confirmed in ¹H NMR spectrum by a doublet appearing at 5.11 ppm corresponding to H-1^{D} with J_{1,2}=2.3 Hz. A second regioselective ring opening step provided efficiently tetrasaccharide acceptor 5 in 95% yield. Hexasaccharide 24, leading to the non-phosphorylated analogue of PS-II repeating unit, was then completed by glycosylation of the tetrasaccharide 5 with the disaccharide donor 3 in moderate 50% yield (Scheme 6). Conversion of the Troc group into acetamide was carried out by basic hydrolysis, which led to concomitant removal of the O-acetyl esters, followed by N-acetylation. Hydrogenation of hexasaccharide intermediate 25 by flow chemistry, utilizing a 10% Pd—C cartridge, gave the first target molecule 1.

Since charged groups are often important epitopes for bacterial saccharides, the role of the phosphate group occurring in the PS-II repeating unit is an important issue to be addressed. Accordingly, the synthesis of phosphorylated hexasaccharide 2 was approached by glycosylation of tetrasaccharide acceptor 5 with disaccharide donor 4 in 62% yield (Scheme 7).

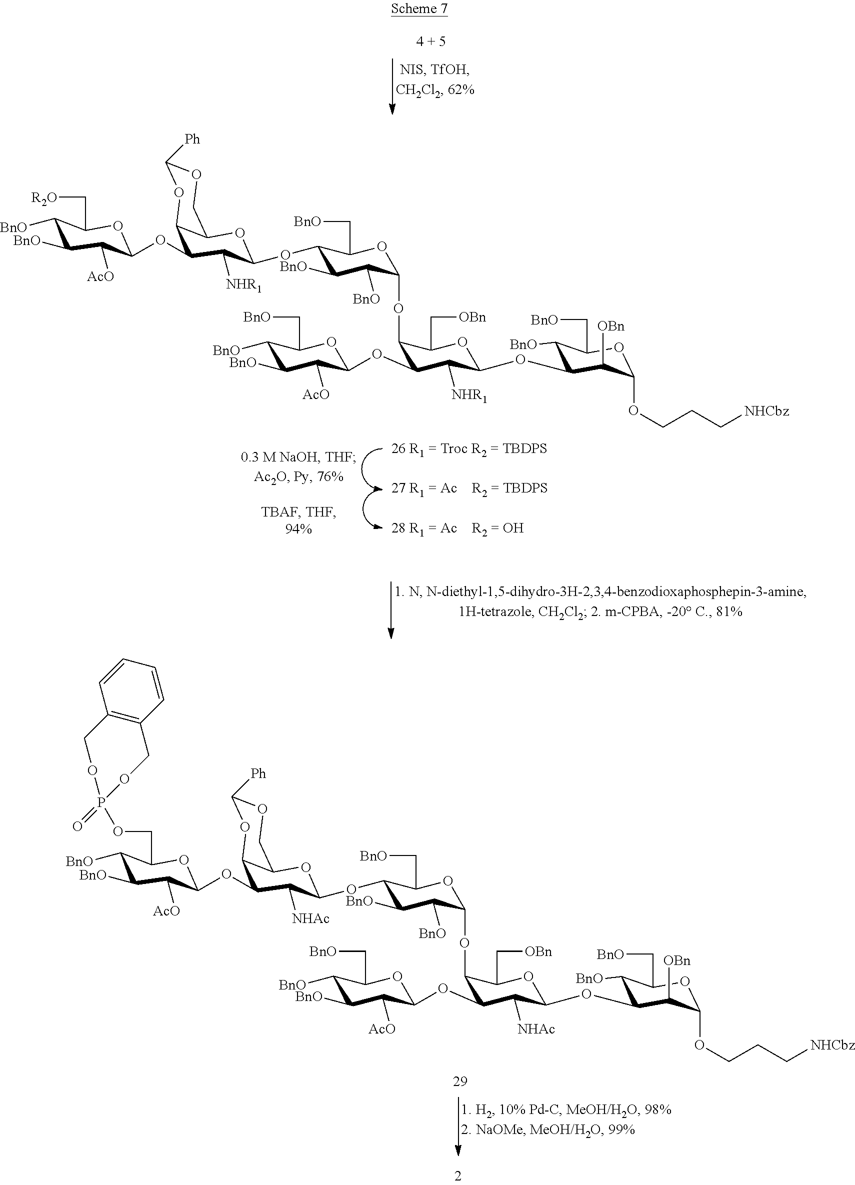

After Troc group removal with 0.3 M NaOH from hexasaccharide 26, the foregoing oligosaccharide was acetylated with acetic anhydride-pyridine to give 27.

Removal of the silyl protection by means of tetrabutylammonium fluoride (TBAF) afforded hexasaccharide 28, suitable for the phosphate group introduction on the primary hydroxyl of C′ unit.

This step was accomplished through reaction with N,N-diethyl-1,5-dihydro-3H-2,3,4-benzodioxaphosphepin-3-amine and 1H-tetrazole, followed by oxidation with m-chloroperbenzoic acid (m-CPBA) [222], furnishing hexasaccharide 29 in 81% yield. A sharp peak in ³¹P NMR spectrum at −0.36 ppm showed the introduction of the protected phosphate, which was confirmed by ESI MS. Final deprotection was performed in nearly quantitative yield by hydrogenation in flow chemistry followed by mild Zemplen transesterification of the acetyl esters. The structures of the purified hexasaccharides 1 and 2 were consistent with the native PS-II repeating unit [212], the main difference being the mannosyl residue which is O-glycosylated with the linker in the synthetic molecules.

Table 1 shows a comparison of NMR δ (ppm) (measured at 400 MHz, 298 K) between hexasaccharide 2 and PS-II repeating unit (PS-II data are reported in italic).

TABLE 1

| | α-Man | β-GalNAc | | β-GalNAc | | α-Glc |
|---|---|---|---|---|---|---|
| | (A) | (B) | β-Glc (C) | (B′) | β-Glc (C′) | (D) |
| H-1 | 4.86 | 4.76 | 4.49 | 4.60 | 4.41 | 4.95 |
| | 5.44 | 4.76 | 4.53 | 4.64 | 4.45 | 4.99 |
| C-1 | 100.6 | 100.5 | 105.6 | 102.5 | 106.0 | 99.6 |
| | 97.0 | 100.7 | 105.5 | 102.3 | 106.0 | 99.6 |
| H-2 | 4.02 | 4.01 | 3.32 | 4.02 | 3.07 | 3.53 |
| | 4.07 | 4.09 | 3.34 | 4.05 | 3.09 | 3.56 |
| C-2 | 68.9 | 53.0 | 74.1 | 53.0 | 74.2 | 72.4 |
| | 69.2 | 53.1 | 73.8 | 52.5 | 74.1 | 72.3 |
| H-3 | 4.02 | 4.00 | 3.49 | 3.90 | 3.45 | 3.97 |
| | 4.07 | 4.00 | 3.49 | 3.93 | 3.49 | 4.01 |
| C-3 | 79.5 | 79.4 | 76.4 | 80.4 | 76.4 | 72.3 |
| | 79.1 | 79.6 | 76.4 | 80.9 | 76.4 | 72.3 |
| H-4 | 3.74 | 4.26 | 3.48 | 4.22 | 3.31 | 3.66 |
| | 3.89 | 4.30 | 3.48 | 4.22 | 3.38 | 3.70 |
| C-4 | 66.2 | 75.6 | 70.6 | 68.4 | 70.6 | 79.8 |
| | 65.6 | 75.5 | 70.2 | 68.7 | 70.7 | 79.6 |
| H-5 | 3.60 | 3.81 | 3.58 | 3.76 | 3.36 | 4.30 |
| | 3.83 | 3.81 | 3.57 | 3.78 | 3.41 | 4.33 |
| C-5 | 65.6 | 76.3 | 76.2 | 76.3 | 76.2 | 71.0 |
| | 74.8 | 76.2 | 75.4 | 76.2 | 76.7 | 70.8 |
| H-6 | 3.69, | 3.67, | 3.90, | 3.76, | 3.95, | 3.66, |
| | 3.80 | 3.90 | 4.21 | 3.90 | 4.00 | 3.82 |
| | n.d. | n.d. | 4.07, | n.d. | 3.74, | 3.68, |
| | | | 4.19 | | 3.93 | 3.84 |
| C-6 | 61.2 | 61.9 | 65.7 | 61.7 | 63.7 | 60.7 |
| | n.d. | n.d. | 65.7 | n.d. | 61.7 | 60.4 |

### General Synthetic Methods

All chemicals were of reagent grade, and were used without further purification. Reactions were monitored by thin-layer chromatography (TLC) on Silica Gel 60 F₂₅₄ (Sigma Aldrich); after exam under UV light, compounds were visualized by heating with 10% (v/v) ethanolic H₂SO₄. In the work up procedures, organic solutions were washed with the amounts of the indicated aqueous solutions, then dried with anhydrous Na₂SO₄, and concentrated under reduced pressure at 30-50° C. on a water bath. Column chromatography was performed on Silica Gel 60 (Sigma Aldrich, 0.040-0.063 nm) or using pre-packed silica cartridges RediSep (Teledyne-Isco, 0.040-0.063 nm) SiliaSep HP (Silicycle, 0.015-0.040 nm) or Supelco (Sigma Aldrich, spherical silica 0.040-0.075 nm). Unless otherwise specified, a gradient 0→100% of the elution mixture was applied in a Combiflash R_{f} (Teledyne-Isco) or Spot II (Armen) instrument. Solvent mixtures less polar than those used for TLC were used at the onset of separation. ¹H NMR spectra were measured at 400 MHz and 298 K with a Bruker Avance^{III} 400 spectrometer; δ_{H} values are reported in ppm, relative to internal Me₄Si (δ_{H}=0.00, CDCl₃); solvent peak for D₂O was calibrated at 4.79 ppm. ¹³C NMR spectra were measured at 100 MHz and 298 K with a Bruker Avance^{III} 400 spectrometer; δ_{C} values are reported in ppm relative to the signal of CDCl₃ (δ_{C}=77.0, CDCl₃) when possible or internal acetone (δ_{C}=30.9, D₂O). Assignments of NMR signals were made by homonuclear and heteronuclear 2-dimensional correlation spectroscopy, run with the software supplied with the spectrometer. Assignment of ¹³C NMR spectra of some compounds was aided by comparison with spectra of related substances reported previously from this laboratory or elsewhere.

When reporting assignments of NMR signals, sugar residues in oligosaccharides are indicated with capital letters, uncertain attributions are denoted “/”. Nuclei associated with the linker are denoted with a prime. Exact masses were measured by electron spray ionization cut-off spectroscopy, using a Q-T of micro Macromass (Waters) instrument. Structures of these compounds follow unequivocally from the mode of synthesis, NMR data and m/z values found in their mass spectra, and their purity was verified by TLC and NMR spectroscopy. Optical rotation was measured with a P-2000 Jasco polarimeter. Hydrogenation reactions were performed in a continuous flow reactor H-Cube (Thalesnano) instrument, using packed catalyst cartridges CatCart.

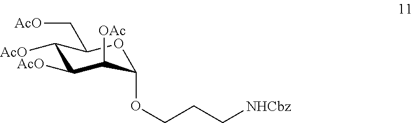

### 3-(Benzyloxycarbonyl)aminopropyl 2,3,4,6-tetra-O-acetyl-α-D-mannopyranoside 11

Trichloroacedimidate donor 10 (31.00 g, 61 mmol) and 3(benzyloxycarbonyl)aminopropanol (16.00 g, 73 mmol) were dissolved in dry dichloromethane (150 ml), under nitrogen atmosphere, then the mixture was cooled to −10° C. and TMSOTf (105 μl, 0.6 mmol) was slowly added. The mixture was stirred overnight allowing it to warm to room temperature, when TLC showed the reaction was complete (1:1 cyclohexane-EtOAc). The mixture was neutralized with triethylamine and concentrated. Chromatography of the crude mixture (9:1→1:1 toluene-EtOAc) gave 14.50 g of foamy product 11 (43%). [α]_{D}²⁴=+17.6 (c 0.55, CHCl₃).

¹H NMR (CDCl₃, 400 MHz): δ=7.39-7.27 (m, 5H, Ph), 5.32 (dd, J_{2,3}=3.5, J_{3,4}=9.9 Hz, 1H, H-3), 5.30-5.20 (m, 2H, H-2, 4), 5.11 (br t, J=5.5 Hz, 1H, NH), 5.09 (s, 2H, CH₂^{Cbz}), 4.76 (s, 1H, H-1), 4.28 (dd, J_{5,6a}=5.3, J_{6a,6b}=12.2 Hz, 1H, H-6a), 4.11 (dd, J_{5,6b}=2.7 Hz, 1H, H-6b), 4.00 (m, 1H, H-5), 3.80 (m, 1H, H-1′a), 3.47 (m, 1H, H-1′b), 3.26 (m, 2H, H-3′), 2.5, 2.08, 2.04, 1.99 (4 s, 12H, 4×CH₃CO), 1.80 (m, 2H, H-2′). ¹³C NMR (CDCl₃, 100 MHz): δ=170.65, 169.83, 169.76, 169.69 (4×CO), 156.42 (CONH), 136.51-127.71 (Ar), 97.60 (C-1), 69.46 (C-2), 69.01 (C-3), 68.46 (C-5), 66.55 (CH₂^{Cbz}), 66.09 (C-4), 65.86 (C-1′), 62.52 (C-6), 38.13 (C-3′), 29.55 (C-2′), 20.97, 20.81, 20.72, 20.65 (CH₃CO). ESI HR-MS (C₂₅H₃₃NO₁₂): m/z=([M+H]⁺ found 540.2088. calcd 540.2081).

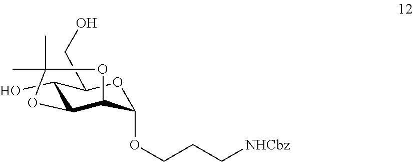

### 3-(Benzyloxycarbonyl)aminopropyl 2,3-O-isopropylidene-α-D-mannopyranoside 12

A solution of the linker equipped Man 11 (8.50 g, 2 mmol) was dissolved in MeOH (200 ml), when 1 M methanolic solution of NaOMe was added until pH was strongly alkaline. The mixture was stirred overnight (TLC, 7:3 cyclohexane-EtOAc), then it was neutralized with Dowex H⁺. After filtration, the filtrate was concentrated and re-dissolved in 1:1 acetone-acetone dimethyl acetale mixture (150 ml). The mixture was stirred for 1 h in the presence of catalytic p-TsOH (0.75 g). After the starting material disappeared (TLC, 7:3 cyclohexane-EtOAc), 75 ml of water were added and stirring was continued for further 6 h. The mixture was concentrated and purified on silica gel (4:1→0:10 toluene-EtOAc) to afford 6.50 g of 2,3-O-isopropylidene product 12 (70%). [α]_{D}²⁴=+16.5 (c 0.23, CHCl₃).

¹H NMR (CDCl₃, 400 MHz): δ=7.41-7.17 (m, 5H, Ph), 5.15-5.05 (m, 3H, CH₂^{Cbz}, NH), 4.99 (s, 1H, H-1), 4.16-4.10 (m, 2H, H-3, 4), 3.89-3.76 (m, 3H, H-2, 5, 1′a), 3.74 (m, 1H, H-6a), 3.62 (m, 1H, H-6b), 3.59 (m, 1H, H-1′b), 3.37 (m, 2H, H-3′), 2.85 (br s, 2H, OH-4, 6), 1.82-1.77 (m, 2H, H-2′), 1.52, 1.35 (2 s, 6H, 2×CH₃). ¹³C NMR (CDCl₃, 100 MHz): δ=156.51 (CONH), 136.46-125.26 (Ar), 109.61 (C(CH₃)₂), 97.33 (C-1), 78.32, 75.55 (C-3, 4), 69.99 (C-6), 69.75 (C-2/5), 65.69 (CH₂^{Cbz}), 65.22 (C-1′), 62.64 (C-2/5), 38.32 (C-3′), 29.59 (C-2′), 27.93, 26.12 (2×CH₃). ESI HR-MS (C₂₀H₂₉NO₈): m/z=([M+Na]⁺ found 434.1797. calcd 434.1791).

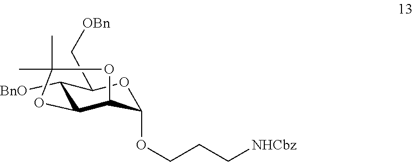

### 3-(Benzyloxycarbonyl)aminopropyl 4,6-di-O-benzyl-2,3-O-isopropylidene-α-D-mannopyranoside 13

To a solution of mannopyranoside 12 (6.50 g, 15.8 mmol) in THF (150 ml) containing 5% of water, powdered NaOH (3.16 g, 79 mmol), BnBr (12.3 ml, 105 mmol) and 18-crown-6 (0.50 g) were added, and the mixture was stirred at room temperature. After 72 h TLC (7:3 cyclohexane-EtOAc) showed the presence of one major spot, so the mixture was concentrated and purified on silica gel (10:0→9:1→0:10 cyclohexane-EtOAc) to give 6.60 g of compound 13 (71%). [α]_{D}²⁴=+53.0 (c 0.28, CHCl₃).

¹H NMR (CDCl₃, 400 MHz): δ=7.40-7.23 (m, 15H, 3×Ph), 5.55 (br t, 1H, J=5.6 Hz, NH), 5.09, 5.03 (2 d, ²J=12.2 Hz, 2H, CH₂^{Cbz}), 5.00 (s, 1H, H-1), 4.82, 4.50 (2 d, ²J=11.5 Hz, 2H, CH₂Ph), 4.58 (s, 2H, CH₂Ph), 4.29 (t, J=6.6. Hz, 1H, H-4), 4.12 (d, J_{3,4}=6.0 Hz, 1H, H-3), 3.87 (m, 1H, H-1′a), 3.76-3.70 (m, 2H, H-6), 3.52-3.48 (m, 2H, H-2, 1′b), 3.44-3.31 (m, 2H, H-5, 3′a), 3.17 (m, 1H, H-3′b), 1.80 (m, 2H, H-2′), 1.49, 1.35 (2 s, 6H, 3×CH₃). ¹³C NMR (CDCl₃, 100 MHz): δ=156.44 (CONH), 137.98-127.54 (Ar), 109.28 (C(CH₃)₂), 97.04 (C-1), 78.80 (C-3), 75.87 (C-4, 5), 73.21, 72.77 (2×CH₂Ph), 69.14 (C-6), 68.64 (C-2), 66.51 (CH₂^{Cbz}), 64.53 (C-1′), 37.84 (C-3′), 29.32 (C-2′), 27.90, 26.21 (2×CH₃). ESI HR-MS (C₃₄H₄₁NO₈): m/z=([M+H]⁺ found 592.2899. calcd 592.2910).

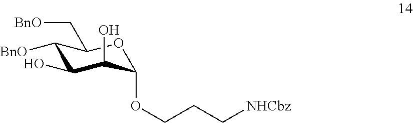

### 3-(Benzyloxycarbonyl)aminopropyl 4,6-di-O-benzyl-α-D-mannopyranoside 14

The compound 13 was dissolved in 90% AcOH—H₂O (100 ml) and stirred overnight at 50° C. When the reaction was complete (TLC, 1:1 cyclohexane-EtOAc) the mixture was concentrated and purified on silica gel (4:1→1:9 toluene-EtOAc) to afford 5.68 g of product 14 (89%). [α]_{D}²⁴=+54.8 (c 0.6, CHCl₃).

¹H NMR (CDCl₃, 400 MHz): δ=7.39-7.13 (m, 15H, 3×Ph), 5.18 (br t, 1H, J=5.2 Hz, NH), 5.09, 5.05 (2 d, ²J=12.0 Hz, 2H, CH₂^{Cbz}), 4.82 (s, 1H, H-1), 4.70, 4.55 (2 d, ²J=11.4 Hz, 2H, CH₂Ph), 4.63, 4.54 (2 d, ²J=12.0 Hz, 2H, CH₂Ph), 3.92-3.85 (m, 2H, H-2, 3), 3.78 (m, 1H, H-1′a), 3.72-3.63 (m, 4H, H-5, 6, incl. t, 3.65 J=9.0 Hz, H-4), 3.47 (m, 1H, H-1′b), 3.33 (m, 1H, H-3′a), 3.21 (m, 1H, H-3′b), 2.52 (m, 2H, OH-2, 3), 1.77 (m, 2H, H-2′). ¹³C NMR (CDCl₃, 100 MHz): δ=156.43 (CONH), 138.09-127.73 (Ar), 99.51 (C-1), 75.80 (C-4), 74.74, 73.47 (2×CH₂Ph), 71.84 (C-3), 71.06 (C-5), 71.00 (C-2), 68.74 (C-6), 66.60 (CH₂^{Cbz}), 65.20 (C-1′), 38.27 (C-3′), 29.44 (C-2′). ESI HR-MS (C₃₁H₃₇NO₈): m/z=([M+H]⁺ found 552.2595. calcd 552.2597).

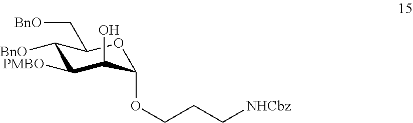

### 3-(Benzyloxycarbonyl)aminopropyl 4,6-di-O-benzyl-3-p-methoxybenzyl-α-D-mannopyranoside 15

A suspension of diol 14 (5.30 g, 10.3 mmol) and Bu₂SnO (3.57 g, 14.4 mmol) in toluene (100 ml) containing pre activated 4 Å MS was stirred under reflux for 1 h. Then temperature was decreased to 60° C. and PMBBr (2.1 ml, 14.4 mmol) was added followed by TBAI (5.3 g, 14.4 mmol). After stirring overnight the reaction was complete (TLC, 7:3 cyclohexane-EtOAc). The mixture was filtered and concentrated. The residue was chromatographed (10:0→9:1 toluene-EtOAc) to give 4.55 g of product 15 (69%). [α]_{D}²⁴=+39.5 (c 0.13, CHCl₃).

¹H NMR (CDCl₃, 400 MHz): δ=7.39-7.22 (m, 15H, 3×Ph), 7.21-6.83 (m, 4H, p-OMe-Ph), 5.31 (br t, 1H, J=5.6 Hz, NH), 5.10, 5.05 (2 d, ²J=11.9 Hz, 2H, CH₂^{Cbz}), 4.86 (s, 1H, H-1), 4.79, 4.46 (2 d, ²J=11.0 Hz, 2H, CH₂Ph), 4.63-4.54 (m, 4H, 2×CH₂Ph), 3.98 (s, 1H, H-2), 3.82-3.61 (m, 9H, H-3, 4, 5, 6, H-1′a, incl. s, 3.79, OMe), 3.50 (m, 1H, H-1′b), 3.36 (m, 1H, H-3′a), 3.23 (m, 1H, H-3′b), 2.49 (s, 1H, OH-2), 1.78 (m, 2H, H-2′). ¹³C NMR (CDCl₃, 100 MHz): δ=156.39 (CONH), 138.10-127.56 (Ar), 113.91 (C_{q}-PMB), 99.15 (C-1), 80.02 (C-3), 75.11 (CH₂Ph), 74.20 (C-4), 73.30, 71.54 (2×CH₂Ph), 71.33 (C-5), 68.82 (C-6), 68.35 (C-2), 66.58 (CH₂^{Cbz}), 65.21 (C-1′). 55.24 (OMe), 38.33 (C-3′), 29.34 (C-2′). ESI HR-MS (C₃₉H₄₅NO₉): m/z=([M+H]⁺ found 672.3155. calcd 672.3173).

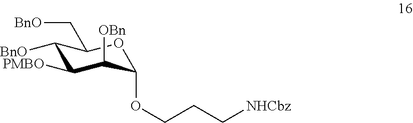

### 3-(Benzyloxycarbonyl)aminopropyl 2,4,6-tri-O-benzyl-3-p-methoxybenzyl-t-D-mannopyranoside 16

To a solution of the 2-hydroxy mannopyranoside 15 (3.60 g, 5.3 mmol) in THF (50 ml) containing 5% of water, powdered NaOH (900 mg, 21.4 mmol), BnBr (2.54 ml, 21.4 mmol) and 18-crown-6 (0.250 mg) were added and the mixture was stirred for 5 d, monitoring by TLC (7:3 cyclohexane-EtOAc). Then the mixture was concentrated and purified on silica gel to give 3.47 g of product 16 (85%). [α]_{D}²⁴=+49.3 (c 0.48, CHCl₃).

¹H NMR (CDCl₃, 400 MHz): δ=7.37-6.83 (m, 24H, 5×Ar), 5.29 (br t, 1H, J=5.6 Hz, NH), 5.11, 5.03 (2 d, ²J=12.2 Hz, 2H, CH₂^{Cbz}), 4.85, 4.44 (2 d, ²J=10.8 Hz, 2H, CH₂Ph), 4.81 (d, J=1.7 Hz, 1H, H-1), 4.79, 4,67 (2 d, ²J=12.4 Hz, 2H, CH₂Ph), 4.59, 4.56 (2 d, ²J=12.5 Hz, 2H, CH₂Ph), 4.51 (s, 2H, CH₂Ph), 3.87-3.61 (m, 10H, H-2, 3, 4, 5, 6, H-1′a, incl. s, 3.79, OMe), 3.42 (m, 1H, H-1′b), 3.32 (m, 1H, H-3′a), 3.17 (m, 1H, H-3′b), 1.73 (m, 2H, H-2′). ¹³C NMR (CDCl₃, 100 MHz): δ=156.40 (CONH), 138.29-127.48 (Ar), 113.71 (C_{q}-PMB), 98.07 (C-1), 79.95 (C-3), 75.05, 74.88, 73.23, 72.70, 72.11, 71.92 (3×CH₂Ph, C-4, 5,6), 69.14 (C-2), 66.55 (CH₂^{Cbz}), 65.07 (C-1′), 55.24 (OMe), 38.25 (C-3′), 29.34 (C-2′). ESI HR-MS (C₄₆H₅₁NO₉): m/z=([M+H]⁺ found 779.3902. calcd 779.3908).

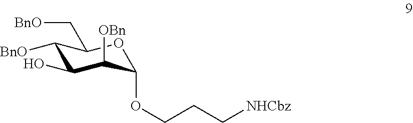

### 3-(Benzyloxycarbonyl)aminopropyl 2,4,6-tri-O-benzyl-α-D-mannopyranoside 9

To a solution of the 3-O-PMB protected sugar 16 (2.00 g, 2.63 mmol) in CH₂Cl₂ (27 ml) moistened with water (3 ml), DDQ (750 mg, 3.31 mmol) was added and the mixture was stirred for 1 h. After 1 h TLC (1:1 cyclohexane-EtOAc) showed the reaction was complete. The mixture was partitioned with 10% sodium thiosulfate, then combined organic layers were concentrated and purified on silica gel (cyclohexane-EtOAc) to afford 1.35 of 9 as solid product (80%). White crystals from EtOAc: m.p. 81-82° C. [α]_{D}²⁴=+32.7 (c 0.22, CHCl₃).

¹H NMR (CDCl₃, 400 MHz): δ=7.40-7.17 (m, 20H, 4×Ph), 5.23 (br t, 1H, J=6.3 Hz, NH), 5.09, 5.04 (2 d, ²J=12.0 Hz, 2H, CH₂^{Cbz}), 4.88 (s, 1H, H-1), 4.83, 4.50 (2 d, ²J=11.0 Hz, 2H, CH₂Ph), 4.73, 4.57 (2 d, ²J=11.3 Hz, 2H, CH₂Ph), 4.60, 4.54 (2 d, ²J=12.4 Hz, 2H, CH₂Ph), 3.94 (ddd, J_{2,3}=3.8, J_{3,4}=J_{3,OH}=9.2 Hz, 1H, H-3), 3.76 (m, 1H, H-1′a), 3.74-3.57 (m, 5H, H-2, 4, 5, 6), 3.45 (m, 1H, H-1′b), 3.34 (m, 1H, H-3′a), 3.18 (m, 1H, H-3′b), 2.32 (d, 1H, OH-3), 1.75 (m, 2H, H-2′). ¹³C NMR (CDCl₃, 100 MHz): d=156.40 (CONH), 138.21-127.55 (Ar), 96.95 (C-1), 78.37 (C-2), 77.20 (C-4), 76.99, 74.82, 73.29 (3×CH₂Ph), 71.83 (C-3), 71.15 (C-5), 69.03 (C-6), 66.54 (CH₂^{Cbz}), 64.99 (C-1′), 38.13 (C-3′), 29.42 (C-2′). ESI HR-MS (C₃₈H₄₃NO₈): m/z=([M+H]⁺ found 642.3033. calcd 642.3067).

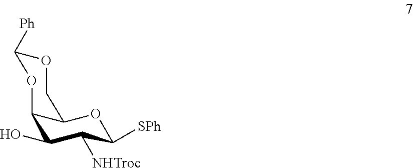

### Phenylthio 4,6-O-benzylidene-2-deoxy-2-(2′,2′,2′-trichloroethoxycarbonylamino)-D-galactopyranoside 7

Phenylthio 3,4,6-tri-O-acetyl-2-deoxy-2-(2′,2′,2′-trichloroethoxycarbonylamino)-β-D-galactopyranoside 17¹ (25.00 g, 45 mmol) was dissolved in MeOH (100 ml), to which 0.25 M methanolic solution of NaOMe was added dropwise at 0° C. until pH=9. After stirring for 3 h at 0° C., the reaction was complete (TLC, cyclohexane-EtOAc 7:3). The mixture was neutralized with Dowex H⁺ and filtrated. The filtrate (45 mmol) was re-dissolved in AcCN (100 ml), and PhCH(OMe)₂ (20.5 ml, 134 mmol) followed by p-TsOH (1.28 g, 0.68 mmol) were added at 0° C. The mixture was stirred for 2 h at ambient temperature, when TLC (cyclohexane-EtOAc 1:1) showed the product was formed. After evaporation of solvent, the residue was chromatographed (cyclohexane-EtOAc) to afford 17.00 g of 7 as solid product (72% over two steps). White crystals from EtOAc: m.p. 171-172° C. [α]_{D}²⁴=−22.7 (c 0.20, CHCl₃).

¹H NMR (CDCl₃, 400 MHz): δ=7.64-7.27 (m, 10H, 2×Ph), 5.69 (m, 1H, NH), 5.54 (s, 1H, PhCH), 4.90 (d, J_{1,2}=10.0 Hz, 1H, H-1), 4.75, 4.69 (2 d, ²J=12.0 Hz, 2H, CH₂^{Troc}), 4.37 (d, J_{6a,6b}=12.3 Hz, 1H, H-6a), 4.22 (d, J_{3,4}=3.2 Hz, 1H, H-4), 4.04 (d, 1H, H-6b), 3.96 (dd, J_{3,2}=9.0, 1H, H-3), 3.69 (q, J_{2,NH}=9.9 Hz, 1H, H-2), 3.56 (s, 1H, H-5). ¹³C NMR (CDCl₃, 100 MHz): δ=154.42 (CONH), 137.46-126.47 (Ar), 101.28 (PhCH), 95.55 (CCl₃), 84.98 (C-1), 75.00 (C-4), 74.59 (CH₂^{Troc}), 71.32 (C-3), 69.91 (C-5), 69.20 (C-6), 53.38 (C-2). ESI HR-MS (C₂₂H₂₂Cl₃NO₅S): m/z=([M+H]⁺ found 534.0302. calcd 534.0312).

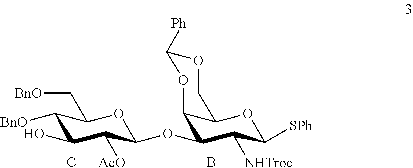

### Phenylthio 2-O-acetyl-3,4,6-tri-O-benzyl-β-D-glucopyranosyl-(1→3)-4,6-O-benzylidene-2-deoxy-2-(2′,2′,2′-trichloroethoxycarbonylamino)-β-D-galactopyranoside 3

Compound 7 can be a thioglicoside (SPh, EtS), imidate (CF₃CNHPh), ether (O-p-methoxyphenyl, O-pentenyl), sylilether (OTBS, OTMS). Amino group could be protected by Troc or any other amino protecting group (Phthalimide, CF₃CO, tetrachlorophthalimide, dimethylmaloyl). Benzyl protecting group can be changed with any other ether or ester (Me, Et, Bz, Piv). Compound 6 can be a donor such as thioglycoside (i.e. SPh, EtS), sulfoxide, imidate (CF₃CNHPh), alogen (F, Cl, Br, I), phosphinite. Benzylidene acetal could be changed with any other ether or ester (Me, Et, Bz, Piv).

To a mixture of acceptor 7 (880 mg, 1.38 mmol) and donor 6 (575 mg, 1.1 mmol) in 2:1 CH₂Cl₂-hexane (12 ml), under nitrogen atmosphere, promoter (0.042 mmol, TMSOTf, NIS-TfOH, BF₃Et₂O) was added at ˜10° C. (−70° C.<t<25° C.). After 15 min TLC (7:3 cyclohexane-EtOAc) showed formation of the product. The mixture was neutralized with a few drops of triethylamine and concentrated.

Chromatography of the residue (toluene-EtOAc) gave the desired disaccharide 3 as a white solid (900 mg, 82%). White crystals from EtOAc: m.p. 149-150° C. [α]_{D}²⁴=+5.8 (c 0.2, CHCl₃).

¹H NMR (CDCl₃, 400 MHz): δ=7.59-7.17 (m, 25H, 5×Ph), 5.44 (d, J_{NH,2}=6.4 Hz, 1H, NH), 5.43 (s, 1H, PhCH), 5.21 (d, J_{1,2}=10.1 Hz, 1H, H-1^{B}), 4.95 (t, J=8.2 Hz, 1H, H-2^{C}), 4.81, 4.50 (2 d, ²J=12.0 Hz, 2H, CH₂Ph), 4.76, 4.62 (2 d×2, ²J=11.3 Hz, 4H, CH₂Ph, CH₂^{Troc}), 4.63, 4.50 (2 d, ²J=11.3 Hz, 2H, CH₂Ph), 4.50 (d, J_{1,2}=8.0 Hz, 1H, H-1^{C}), 4.39-4.35 (m, 2H, H-3^{B}, 4^{B}), 4.27 (d, J_{6a,6b}=12.0 Hz, 1H, H-6a^{B}), 3.82 (d, 1H, H-6b^{B}), 3.70 (dd, J_{5,6a}=1.7, J_{6a,6b}=10.3 Hz, 1H, H-6a^{C}), 3.71-3.52 (m, 4H, H-2^{B}, 3^{C}, 4^{C}, 6b^{C}), 3.43-3.39 (m, 2H, H-5^{B,C}), 1.89 (s, 3H, CH₃CO). ¹³C NMR (CDCl₃, 100 MHz): δ=169.48 (CO), 153.68 (CONH), 137.96-125.28 (Ar), 100.68 (CHPh), 100.59 (C-1^{C}), 95.54 (CCl₃), 84.15 (C-1^{B}), 83.01, 77.90 (C-3^{C}, 4^{C}), 75.86, 75.82 (C-3^{B}, 4^{B}), 75.03 (2×CH₂), 74.47 (C-5^{C}), 74.27, 73.55 (2×CH₂), 72.82 (C-2^{C}), 69.96 (C-5^{B}), 69.20 (C-6^{B,C}), 51.22 (C-2^{B}), 20.84 (CH₃CO). ESI HR-MS (C₅₁H₅₂Cl₃NO₁₂S): m/z=([M+Na]⁺ found 1030.2217. calcd 1030.2247); ([M+K]⁺ found 1046.1865; calcd 1046.1913).

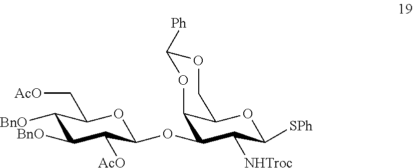

### Phenylthio 2,6-di-O-acetyl-3,4-di-O-benzyl-β-D-glucopyranosyl-(1→3)-4,6-O-benzylidene-2-deoxy-2-(2′,2′,2′-trichloroethoxycarbonylamino)-β-D-galactopyranoside 19

Compound 7 can be a thioglicoside (SPh, EtS), imidate (CF₃CNHPh), ether (O-p-methoxyphenyl, O-pentenyl), sylilether (OTBS, OTMS). Amino group could be protected by Troc or any other amino protecting group (Phthalimide, CF₃CO, tetrachlrophthalimide, dimethylmaloyl). Benzyl protecting group can be changed with any other ether or ester (Me, Et, Bz, Piv). Compound 18 can be a donor such as thioglycoside (i.e. SPh, EtS), sulfoxide, imidate (CF₃CNHPh), alogen (F, Cl, Br, I), phosphonate. Benzylidene acetal could be changed with any other ether or ester (Me, Et, Bz, Piv).

To a solution of acceptor 7 (571 mg, 1.1 mmol) and donor 18 (880 mg, 1.38 mmol) in 1:1 CH₂Cl₂-hexane (30 ml), under nitrogen atmosphere, promoter (0.014 mmol TMSOTf, NIS-TfOH, BF₃Et₂O) was added at −30° C. (−70° C.<t<25° C.). After 15 min the mixture became cloudy and the flask was brought to ambient temperature. TLC (cyclohexane-EtOAc 3:2) showed the reaction had taken place. The reaction mixture was neutralized with few drops of triethylamine, and concentrated. The residue was chromatographed on silica gel to afford 530 mg of disaccharide 19 (52%). [α]_{D}²⁴=+23.94 (c 0.23, CHCl₃).

¹H NMR (CDCl₃, 400 MHz): J=7.65-7.17 (m, 20H, 4×Ph), 5.53 (s, 1H, PhCH), 5.37 (d, J_{NH,2}=6.9 Hz, 1H, NH), 5.28 (d, J_{1,2}=10.0 Hz, 1H, H-1^{B}), 4.94 (t, J=8.8 Hz, 1H, H-2^{C}), 4.82-4.75 (m, 3H, 3×HCH), 4.65-4.55 (m, 5H, H-6a^{C}), 4.40-4.33 (m, 3H, H-3^{B}, 4^{B}, 6a^{B}), 4.06-4.01 (m, 2H, H-6^{B}, 6b^{C}), 3.61-3.51 (m, 4H, H-2^{B}, 3^{C}, 4^{C}, 5^{B}), 3.44 (m, 1H, H-5^{C}), 2.00, 1.91 (2×s, 6H, 2×CH₃CO). ¹³C NMR (CDCl₃, 100 MHz): δ=170.57, 169.48 (2×CO), 153.63 (CONH), 138.24-126.08 (Ar), 101.23 (CHPh), 100.51 (C-1^{C}), 95.48 (CCl₃), 84.06 (C-1^{B}), 82.75, 77.20 (C-3^{C}, 4^{C}), 75.64, 75.24 (C₁₋₃^{B}, 4^{B}), 75.09, 75.00, 74.15 (3×CH₂), 73.09 (C-5^{C}), 72.76 (C-2^{C}), 70.02 (C-5^{B}), 69.23 (C-6^{B}), 62.20 (C-6^{C}), 51.13 (C-2^{B}), 20.84, 20.80 (2×CH₃CO). ESI HR-MS (C₄₆H₄₈Cl₃NO₁₃S): m/z=([M+H]⁺ found 960.1965. calcd 960.1990).

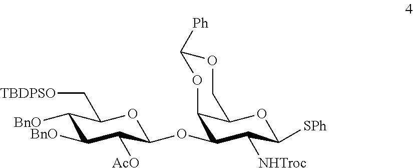

### Phenylthio 2-O-acetyl-3,4-di-O-benzyl6-O-tert-butyldiphenylsilyl-β-D-glucopyranosyl-(1→3)-4,6-O-benzylidene-2-deoxy-2-(2′,2′,2′-trichloroethoxycarbonylamino)-β-D-galactopyranoside 4

A solution of disaccharide 19 (830 mg, 0.87 mmol) in MeOH (50 ml) was made alkaline (pH=9) by dropwise addition of 0.25 M methanolic solution of NaOMe. The mixture was stirred overnight at 0° C., when TLC (3:2 cyclohexane-EtOAc) showed the formation of a lower moving spot. The mixture was neutralized with Dowex H⁺ and filtrated. The filtrate was concentrated and purified on silica gel to afford 575 mg of 6-de-O-acetylated product 20 (73%).

### Phenylthio2-O-acetyl-3,4-di-O-benzyl-f-D-glucopyranosyl-(1→3)-4,6-O-benzylidene-2-deoxy-2-(2′,2′,2′-trichloroethoxycarbonylamino)-β-D-galactopyranoside 20

¹H NMR (CDCl₃, 400 MHz): δ=7.65-7.21 (m, 20H, 4×Ph), 5.50 (s, 1H, PhCH), 5.33 (d, J_{NH,2}=7.3 Hz, 1H, NH), 5.26 (d, J_{1,2}=9.6 Hz, 1H, H-1^{B}), 4.94 (t, J=8.5 Hz, 1H, H-2^{C}), 4.87-4.55 (m, 7H, 2×CH₂Ph, CH₂^{Troc}, H-1^{C}), 4.51 (br d, J_{2,3}=10.6 Hz, 1H, H-3^{B}), 4.37-4.33 (m, 2H, H-4^{B}, 6a^{B}), 4.02 (d, J_{6a,6b}=12.1 Hz, 1H, 6b^{B}), 3.74-3.62 (m, 3H, H-2^{B}, 6^{B}), 3.59-3.47 (m, 3H, H-3^{C}, 4^{C}, 5^{B}), 3.30 (m, 1H, H-5^{C}), 1.87 (s, 3H, CH₃CO). ESI HR-MS (C₄₄H₄₆Cl₃NO₁₂S): m/z=([M+H]⁺ found 918.1892. calcd 918.1885).

To a solution of the 6-OH disaccharide 20 (550 mg, 0.59 mmol) in DMF (4 ml), TBDPSCl (0.31 ml, 1.2 mmol) and imidazole (82 mg, 1.2 mmol) were added. TBDPSCl can be replaced by any other sylil chloride or ester (chloroacetate, bromoacetate, levulinic) After stirring for 24 h TLC (4:1 cyclohexane-EtOAc) showed the reaction was complete The mixture was concentrated, and the residue was purified on silica gel (cyclohexane-EtOAc) to give 630 mg of foamy product 4 (92%). [α]_{D}²⁴=−12.90 (c 0.11, CHCl₃).

¹H NMR (CDCl₃, 400 MHz): δ=7.71-7.03 (m, 30H, 6×Ph), 5.43 (s, 1H, PhCH), 5.18 (d, J_{1,2}=10.0 Hz, 1H, H-1^{B}), 5.17 (d, J_{NH,2}=7.2 Hz, 1H, NH), 4.99 (t, J=8.1 Hz, 1H, H-2^{C}), 4.80, 4.64 (2 d, ²J=11.2 Hz, 2H, CH₂Ph), 4.80, 4.50 (2 d, ²=10.5 Hz, 2H, CH₂Ph), 4.76, 4.62 (2 d, ²J=11.2 Hz, 2H, CH₂^{Troc}), 4.61 (d, J_{1,2}=7.4, 1H, H-1c), 4.40 (br s, 1H, H-4^{B}), 4.38 (br d, J_{2,3}=10.6 Hz, 1H, H-3^{B}), 4.25 (d, J_{6a,6b}=12.1 Hz, 1H, 6a^{B}), 4.03 (d, J_{6a,6b}=10.5 Hz, 1H, 6a^{C}), 3.89 (dd, J_{5,6}=5.4 Hz, 1H, 6b^{C}), 3.77 (d, 1H, 6b^{B}), 3.70-3.57 (m, 3H, H-2^{B}, 3^{C}, 4^{C}), 3.49 (m, 1H, H-5^{C}), 3.34 (s, 1H, H-5^{B}), 1.94 (s, 3H, CH₃CO), 1.10 (s, 9H, t-Bu). ¹³C NMR (CDCl₃, 100 MHz): δ=169.66 (CO), 153.69 (CONH), 137.89-126.36 (Ar, C(CH₃)₃), 101.80 (C-1^{C}), 100.49 (CHPh), 95.47 (CCl₃), 84.29 (C-1^{B}), 82.96, 77.62 (C-3^{C}, 4^{C}), 76.27, (C-5^{C}), 76.12, 75.85 (C-3^{B}, 4^{B}), 75.09, 74.91, 74.17 (3×CH₂), 72.94 (C-2^{C}), 70.00 (C-5^{B}), 69.90 (C-6^{B}), 63.01 (C-6^{C}), 51.49 (C-2^{B}), 26.88 (t-Bu), 20.87 (CH₃CO). ESI HR-MS (C₆₀H₆₄Cl₃NO₁₂SSi): m/z=([M+H]⁺ found 1178.2897. calcd 1178.2882).

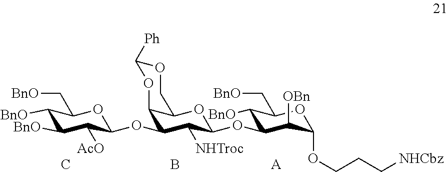

### 3-(Benzyloxycarbonyl)aminopropyl-2-O-acetyl-3,4,6-tri-O-benzyl-β-D-glucopyranosyl-(1→3)-4,6-O-benzylidene-2-deoxy-2-(2′,2′,2′-trichloroethoxycarbonylamino)-β-D-galactopyranosyl-(1→3)-2,4,6-tri-O-benzyl-α-D-mannopyranoside 21

In acceptor 9 the anomeric position can be present an alkyl or aromatic ether (OMe, EtO, PhO) or any other linker to allow conjugation to a carrier protein.

A solution of acceptor 9 (1.18 g, 1.84 mmol) and donor 3 (2.26 g, 2.27 mmol) was stirred at −40° C. in presence of 4 Å MS, under nitrogen atmosphere. After addition of NIS (0.53 g, 2.33 mmol) and TfOH (38.6 μl, 0.44 mmol) the mixture turned immediately red and TLC (7:3 cyclohexane-EtOAc) showed that a new spot was formed. Any other promoter could be employed TMSOTf, NIS-TfOH, BF₃Et₂O) with 70° C.<t<25° C.). The reaction mixture was washed with 10% NaS₂O₃-aq NaHCO₃. Combined organic layers were dried on Na₂SO₄, filtered and purified on silica gel (cyclohexane-EtOAc) to yield trisaccharide 21 (2.2 g, 77%). [α]_{D}²⁴=+46.5 (c=0.05, CHCl₃)

¹H NMR (CDCl₃, 400 MHz): δ=7.53-7.13 (m, 40H, 8×Ph), 5.59 (d, J_{NH,2}=7.0 Hz, 1H, NH^{B}), 5.43 (s, 1H, PhCH), 5.37 (br t, J=5.2 Hz, 1H, NH^{Cbz}), 5.05 (br s, 2H, CH₂^{Cbz}), 5.03 (d, J_{1,2}=8.7 Hz, 1H, H-1^{B}), 5.02-4.95 (m, 2H, H-2^{C}, HCH), 4.78-4.74 (m, 3H, 2×HCH, incl. s, 4.75, H-1^{A}), 4.63-4.46 (m, 11H, 10×HCH, H-1^{C}), 4.42-4.34 (m, 2H, HCH, H-3^{B}), 4.28 (d, J_{3,4}=2.6 Hz, 1H, H-4^{B}), 4.11 (m, 1H, H-3^{A}), 3.99 (d, J_{6a,6b}=12.1 Hz, 1H, H-6a^{B}), 3.89-3.48 (m, 12H, H-2^{A,B}, 3^{C}, 4^{A,C}. 5^{A}, 6a^{A,C}, 6b^{A,B,C}, 1′a), 3.43 (m, 1H, H-1′b), 3.36-3.27 (m, 2H, H-5^{C}, 3′a), 3.22 (s, 1H, H-5^{B}), 3.17 (m, 1H, H-3b′), 1.89 (s, 3H, CH₃CO), 1.72 (m, 2H, H-2′). ¹³C NMR (CDCl₃, 100 MHz): δ=169.55 (CO), 156.45, 153.80 (2×CONH), 138.44-126.32 (Ar), 101.54 (C-1^{C}), 100.64 (CHPh), 99.91 (C-1^{B}), 97.64 (C-1^{A}), 95.40 (CCl₃), 82.91 (C-3/4^{C}), 78.69 (C-3^{A}), 77.91 (C-3/4^{C}), 75.71, 75.82 (C-2^{A}, 4^{B}), 74.96, 74.92, 74.61, 74.43, 74.31, 74.10 (4×CH₂, C-4^{A}, C-3^{B}), 73.44 (2×CH₂), 73.10 (C-2^{C}), 72.72, 72.46 (2×CH₂), 72.00 (C-5^{A}), 69.17 (C-6^{A}), 68.76 (C-5^{C}), 68.17, 67.96 (C-6^{B,C}), 66.48 (C-5^{B}), 64.74 (C-1′), 53.68 (C-2^{B}), 37.87 (C-3′), 29.40 (C-2′), 20.83 (CH₃CO). ESI HR-MS (C₈₃H₈₉Cl₃N₂O₂₀): m/z=([M+Na]⁺ found 1561.4944 calcd 1561.4972); ([M+K]⁺ found 1577.4655 calcd 1577.4711).

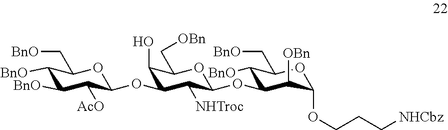

### 3-(Benzyloxycarbonyl)aminopropyl-2-O-acetyl-3,4,6-tri-O-benzyl-β-D-glucopyranosyl-(1→3)-6-O-benzyl-2-deoxy-2-(2′,2′,2′-trichloroethoxycarbonylamin)-β-D-galactopyranosyl-(1→3)-2,4,6-tri-O-benzyl-α-D-mannopyranoside 22

The starting trisaccharide 21 (330 mg, 0.2 mmol) was dissolved in dry acetonitrile (30 ml) under nitrogen atmosphere and treated with trimethylamineborane (83 mg, 1.08 mmol) or triethylsilane or NaCNBH₃ and BF₃-Et₂O (0.176 ml, 1.08 mmol) or any other acid (TFA, HCl) at 0° C. After stirring for 1 h at 0° C., the mixture was quenched with triethylamine and MeOH and concentrated. Chromatography of the residue (cyclohexane-EtOAc) afforded 265 mg of syrupy product 22 (80%). [α]_{D}²⁴=+50.06 (c=0.36, CHCl₃)

¹H NMR (CDCl₃, 400 MHz): δ=7.38-7.17 (m, 40H, 8×Ph), 5.40 (br t, J=5.2 Hz, 1H, NH^{Cbz}), 5.04 (br s, 2H, CH₂^{Cbz}), 4.97 (t, J=8.4 Hz, 1H, H-2^{C}), 4.91 (d, J_{NH,2}=6.6 Hz, 1H, NH^{B}), 4.80 (d, J_{1,2}=7.5 Hz, 1H, H-1^{B}), 4.79 (s, 1H, H-1^{A}), 4.77-4.74 (m, 4H, HCH), 4.68-4.62 (m, 2H, HCH), 4.55-4.33 (m, 11H, 10×HCH, H-1^{C}), 4.11-4.08 (m, 3H, H-3^{B}, H-4^{B}, H-3^{A}), 3.89-3.42 (m, 16H, H-2^{A,B}, 3^{C}, 4^{A,C}, 5^{A,B,C}, 6a^{A,B,C}, 6^{A,B,C}, H-1′), 3.30 (m, 1H, H-3′a), 3.14 (m, 1H, H-3′b), 2.73 (br s, 1H, OH-4^{B}), 1.91 (s, 3H, CH₃CO), 1.70 (m, 2H, H-2′). ¹³C NMR (CDCl₃, 100 MHz): δ=169.44 (CO), 156.46, 153.64 (2×CONH), 138.78-127.47 (Ar), 101.49 (C-1^{C}), 99.34 (C-1^{B}), 98.17 (C-1^{A}), 95.34 (CCl₃), 82.61 (C-3^{C}), 78.32 (C-3^{B}), 77.65 (C-4^{B}), 77.65 (C-4^{C}), 77.18 (C-2^{A}), 75.04 (2×CH₂), 75.33 (C-5^{B}), 75.00 (C-5^{A/C}), 74.86, 74.21, 73.81, (3×CH₂), 73.41 (C-4^{A}), 73.11 (CH₂), 72.74 (C-2^{C}), 72.60 (2×CH₂), 71.87 (C-5^{A/C}), 69.39, 69.17, 68.57 (3×C-6^{A/B/C}), 67.75 (C-3^{A}), 66.42 (CH₂^{Cbz}), 64.64 (C-1′), 54.36 (C-2^{B}), 37.86 (C-3′), 29.35 (C-2′), 20.81 (CH₃CO). ESI HR-MS (C₈₃H₉₁Cl₃N₂O₂₀): m/z=([M+Na]⁺ found 1563.5134. calcd 1563.5128); ([M+K]⁺ found 1579.4945. calcd 1579.4868).

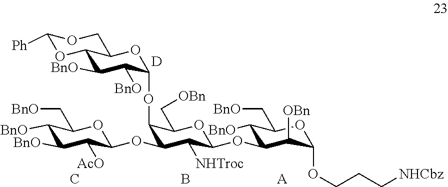

### 3-(Benzyloxycarbonyl)aminopropyl-2-O-acetyl-3,4,6-tri-O-benzyl-β-D-glucopyranosyl-(1→3)-[6-O-benzyl-2-deoxy-2-(2′,2′,2′-trichloroethoxycarbonylamino)-β-D-galactopyranosyl-(1→3)-2,3-di-O-benzyl-4,6-O-benzylidene-α-D-glucopyranosyl-(1→4)]-2,4,6-tri-O-benzyl-α-D-mannopyranoside 23

Donor 8 can be a thiolgicoside thioglycoside (i.e. SPh, EtS), sulfoxide, imidate (CF₃CNHPh, CCl₃CNH), alogen (F, Cl, Br, I), phosphinite. Benzylidene acetal could be changed with any other ether or ester (Me, Et, Bz, Piv). Position 5 can be protected with a selective removable group (Fmoc, levulinic, bromoacetate, chlroacetate). Any other order of assembling (i.e. A+B+C+D, C+B+D+A, etc.) is possible.

A solution of acceptor 22 (600 mg, 0.389 mmol) and donor 8 (287 mg, 0.583 mmol) was stirred at 0° C. in presence of 4 Å MS, under nitrogen atmosphere. After addiction of NIS (131 mg, 0.583 mmol) and TfOH (13.8 μl, 0.156 mmol) the mixture turned immediately red and the reaction mixture was stirred at room temperature. After 5 h further portions of NIS (20 mg, 0.089 mmol) and TfOH (3.4 μl, 0.039) were added to complete the reaction. Any other promoter could be employed (TMSOTf, NIS-TfOH, BF₃Et₂O) with 70° C.<t<25° C.). After further 3 h TLC (7:3 toluene-EtOAc) showed that the reaction was complete, so triethylamine was added to neutralize the reaction and the mixture was concentrated. The residue was purified on silica gel (95:5→1:1 Toluene-EtOAc) to yield 680 mg of tetrasaccaride 23 (89%). [α]_{D}²⁴=+33.8 (c 0.80, CHCl₃).

¹H NMR (CDCl₃, 400 MHz): δ=7.50-7.11 (m, 55H, 11×Ph), 5.53 (s, 1H, PhCH), 5.42 (m, 1H, NH^{Cbz}), 5.11 (d, J=2.3 Hz, 1H, H-1^{D}), 5.04-5.00 (m, 4H, NH^{B}, CH₂^{Cbz}, H-2^{C}), 4.84 (d, J_{1,2}=7.84 Hz, 1H, H-1^{B}), 4.83 (d, ²J=11.7 Hz, 1H, HCH), 4.78 (s, 1H, H-1^{A}), 4.75-4.71 (m, 2H, HCH), 4.67-4.39 (m, 16H, 14×HCH, H-3^{A}, H-1^{C}), 4.32 (d, ²J=12.04 Hz, 1H, HCH), 4.25-4.03 (m, 8H, H-3^{B,D}, 4^{A,B}, 6^{D/B}, CH₂^{Troc}), 3.76-3.27 (m, 18H, H-2^{A,B,D}, 3^{C}, 4^{C,D}, 5^{A,B,C,D}, 6^{A,B/D,C}, 1′), 3.28 (m, 1H, H-3′a), 3.11 (m, 1H, H-3′b), 1.92 (s, 3H, CH₃CO), 1.71 (m, 2H, H-2′). ¹³C NMR (CDCl₃, 100 MHz): δ=169.04 (CO), 156.34, 153.65 (2×CONH), 138.81-123.48 (Ar), 101.81 (C-1^{C}), 101.49 (CHPh), 100.18 (C-1^{B}), 99.12 (C-1^{D}), 98.12 (C-1^{A}), 95.40 (CCl₃), 82.82 (C-3^{C}), 82.52, 80.11 (C-2^{D}), 78.54 (C-3^{D}), 78.14 (C-4^{B}), 77.53, 77.32, 77.02 (C-3^{B}), 76.52, 76.19, 74.87, 74.81, 74.78 (3×CH₂), 74.54, 73.93 (CH₂), 73.89, 73.55, 73.21, 73.02, 72.90, 72.81 (5×CH₂), 72.52 (C-2^{C}), 72.36 (CH₂^{Troc}), 71.70 (C-3^{A}), 69.33, 69.09, 68.52 (C-6^{A/B/C/D}), 66.28 (CH₂^{Cbz}), 64.32 (C-1′), 62.85 (C-4^{A}), 54.78 (C-2^{B}), 37.52 (C-3′), 29.18 (C-2′), 20.84 (CH₃CO).

ESI HR-MS (C₁₁₀H₁₁₇Cl₃N₂O₂₅): m/z=([M+Na]⁺ found 1993.6755. calcd 1993.6909); ([M+K]⁺ found 2009.6423. calcd 2009.6648).

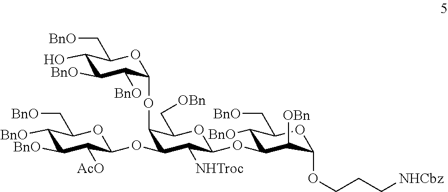

### 3-(Benzyloxycarbonyl)aminopropyl-2-O-acetyl-3,4,6-tri-O-benzyl-β-D-glucopyranosyl-(1→3)-6-[O-benzyl-2-deoxy-2-(2′,2′,2′-trichloroethoxycarbonylamino)-β-D-galactopyranosyl-(1→3)-2,3-tri-O-benzyl-α-D-glucopyranosyl-(1→4))]-2,4,6-tri-O-benzyl-α-D-mannopyranoside 5

The starting tetrasaccharide 23 (230 mg, 0.117 mmol) was dissolved in dry acetonitrile (26 ml) under nitrogen atmosphere and treated with trimethylamineborane (43 mg, 0.584 mmol) or triethylsilane or NaCNBH₃ and BF₃Et₂O (0.072 ml, 0.584 mmol) or any other acid (TFA, HCl) at 0° C. After 1 h at 0° C., the mixture was quenched with triethylamine and MeOH and concentrated. Chromatography of the residue (cyclohexane-EtOAc) afforded 220 mg of product 5 (95%). [α]_{D}²⁵=+58.08 (c=0.13, CHCl₃ and BF₃.Et₂O (0.176 ml, 1.08 mmol)

¹H NMR (CDCl₃, 400 MHz): δ=7.29-7.08 (m, 55H, 11×Ph), 5.36 (m, 1H, NH^{Cbz}), 5.02 (d, J_{1,2}=2.5 Hz, 1H, H-1^{D}), 4.96-4.92 (m, 3H, NH^{B}, CH₂^{Cbz}), 4.74-4.69 (m, 9H, 6×HCH, H-2^{C}, incl. d, 4.72, J_{1,2}=2.7 Hz, H-1^{A}; d, 4.70 J_{1,2}=8.5 Hz, H-1^{B}), 4.55-4.25 (m, 18H, 16×HCH, H-5^{D}, incl. d, 4.34, J_{1,2}=7.7 Hz, H-1^{C}), 4.18 (br s, 1H, H-4^{B}), 4.16-4.03 (m, 4H, 2×HCH, H-3^{B}, H-3^{A}), 3.85-3.37 (m, 19H, H-2^{A,B,D}, 3^{C,D}, 4^{A,C,D}, 5^{A,B,C}, 6^{A,B,C}, 1′) 3.23 (m, 1H, H-3′a), 3.07 (m, 1H, H-3′b), 2.86 (br s, 1H, OH-4^{D}), 1.85 (s, 3H, CH₃CO), 1.64 (m, 2H, H-2′). ¹³C NMR (CDCl₃, 100 MHz): 6^{=169.76} (CO), 156.46, 153.80 (2×CONH), 139.14-127.29 (Ar), 101.60 (C-1^{C}), 99.71 (C-1^{B}), 98.11 (C-1^{A}), 97.47 (C-1^{D}), 95.51 (CCl₃), 82.63 (C-3^{C}), 81.78 (C₄^{D}), 79.63, 77.83, 77.50 (C-3^{A}), 76.80, 76.01 (C-3^{B}), 75.33 (C-2^{D}), 75.04, 75.03 (3×CH₂), 74.97, 73.92, 73.95 (C-4^{B}, 5^{B}), 73.58, 73.55, 73.26, 73.18, 73.05 (6×CH₂), 72.92 (C-2^{C}), 72.83, 72.40 (2×CH₂), 71.82 (C-4^{D}), 71.70, 70.55 (C-5^{D}), 69.57, 69.49, 68.60, 68.36 (C-6^{A,B,C,D}), 66.46 (CH₂^{Cbz}), 64.39 (C-1′), 54.95 (C-2^{B}), 37.63 (C-3′), 29.31 (C-2′), 20.95 (CH₃CO). ESI HR-MS (C₁₁₀H₁₁₉Cl₃N₂O₂₅): m/z=([M+Na]⁺ found 1995.7106. calcd 1995.7065); ([M+K]⁺ found 2011.6824; calcd 2011.6805).

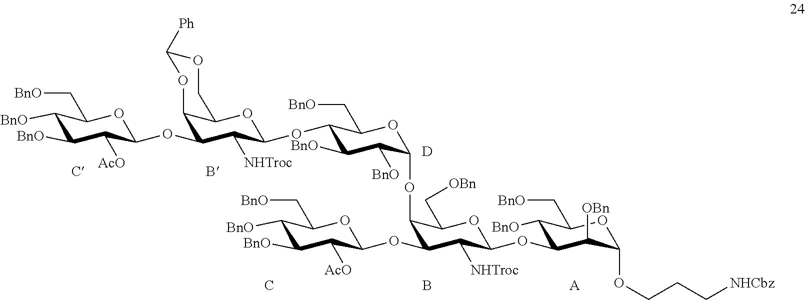

### 3-(Benzyloxycarbonyl)aminopropyl-2-O-acetyl-3,4,6-tri-O-benzyl-β-D-glucopyranosyl-(1→3)-[2-O-acetyl-3,4,6-tri-O-benzyl-β-D-glucopyranosyl-(1→3)-4,6-O-benzilidene-2-deoxy-2-(2′,2′,2′-trichloroethoxycarbonylamino)-β-D-galactopyranosyl-(1→4)-2,3,6-tri-O-benzyl-α-D-glucopyranosyl-(1→4)]-6-O-benzyl-2-deoxy-2-(2′,2′,2′-trichloroethoxycarbonylamino)-β-D-galactopyranosyl-(1→3)-2,4,6-tri-O-benzyl-α-D-mannopyranoside 24

Donor 3 can be a thioglycoside (i.e. SPh, EtS), sulfoxide, imidate (CF₃CNHPh, CCl₃CNH), alogen (F, Cl, Br, I), phosphinite.

A solution of acceptor 5 (100 mg, 0.051 mmol) and donor 3 (83 mg, 0.083 mmol) was stirred at 0° C. in the presence of 4 Å MS, under nitrogen atmosphere. After addiction of NIS (18 mg, 0.082 mmol) and TfOH (18 ul, 0.02 mmol) the mixture turned immediately red and the reaction mixture was stirred at room temperature for 8 h. Any other promoter could be employed (TMSOTf, NIS-TfOH, BF₃Et₂O) with 70° C.<t<25° C.). When TLC (Toluene-EtOH 9:1) showed the reaction was complete, it was neutralized with a drop of triethylamine and concentrated. The residue was purified on silica gel (95:5→1:1 toluene-AcOEt) to yield 75 mg of hexasaccaride (50%) 24. [α]_{D}²⁴=+23.5 (c 0.25, CHCl₃).

¹H NMR (CDCl₃, 400 MHz): δ=7.44-7.14 (m, 75H, 15×Ph), 5.68, 5.57 (2 m, 2H, 2×NH^{B,B′}), 5.40 (m, 1H, NH^{Cbz}), 5.37 (s, 1H, PhCH), 5.16 (d, J_{1,2}=1.2 Hz, 1H, H-1^{D}), 5.10-4.87 (m, 9H, CH₂^{Cbz}, 2×HCH, H-2^{C,C′}, H-1^{A,B,B′}), 4.82-4.70 (m, 8H, 8×HCH), 4.64-4.37 (m, 20H, 18×HCH, H-1^{C,C′}), 4.31-3.43 (m, 38H, 2×HCH, H-2^{A,B,B′,C,C′,D}, 3^{A,B,B′,C,C′,D}, 4^{A,B,B′C,C′,D}, 5^{A,B,B′,C,C′,D}, 6^{A,B,B′,C,C′,D}, 1′), 3.23 (m, 1H, H-3′a), 3.14 (m, 1H, H-3′b), 1.91, 1.87 (2×s, 6H, 2×CH₃CO), 1.72 (m, 2H, H-2′). ¹³C NMR (CDCl₃, 100 MHz): δ=169.82, 169.45 (2×CO), 156.42, 153.76 (3×CONH), 139.14-125.24 (Ar), 102.09, 101.96 (C-1^{C,C′}), 100.28 (CHPh), 99.62, 98.84 (C-1^{B,B′}), 97.97 (C-1^{A,D}), 97.97 (C-1^{D}), 97.78, 95.50 (2×CCl₃), 83.51, 82.94, 82.62, 81.01, 80.60, 80.47, 80.27, 79.99, 79.79, 78.86, 77.91, 77.89, 77.32, 77.09, 76.68, 75.70, 74.93, 74.64, 73.94, 73.45, 73.30, 73.16, 73.08, 72.97, 72.42 (C-2^{C/C′}), 71.83 (C-2^{C/C′}), 70.83, 69.50, 69.20, 68.74, 68.62, 68.00, 67.12, 66.42 (CH₂^{Cbz}), 66.10, 64.13 (C-1′), 54.83 (C-2^{B/B′}), 54.50 (C-2^{B/B′}), 37.47 (C-3′), 29.19 (C-2′), 21.40, 20.79 (2×CH₃CO). ESI HR-MS (C₁₅₅H₁₆₅Cl₆N₃O₃₇): m/z=([M+Na]⁺ found 2892.9521. calcd 2892.9151).

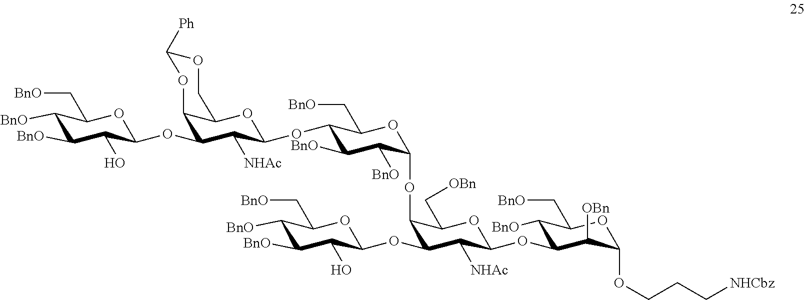

### 3-(Benzyloxycarbonyl)aminopropyl 3,4,6-tri-O-benzyl-β-D-glucopyranosyl-(1→3)-[3,4,6-tri-O-benzyl-β-D-glucopyranosyl-(1→3)-4,6-O-benzilidene-2-acetamido-2-deoxy-β-D-galactopyranosyl-(1→4)-2,3,6-tri-O-benzyl-α-D-glucopyranosyl-(1→4)]-2-acetamido-6-O-benzyl-2-deoxy-β-D-galactopyranosyl-(1→3)-2,4,6-tri-O-benzyl-α-D-mannopyranoside 25

The hexasaccharide 24 (87 mg, 0.032 mmol) was dissolved in THF (5 ml) to which 3 M NaOH (0.5 ml) was added. After refluxing for 2 d (TLC, 7:3 cyclohexane-EtOAc), the mixture was neutralized with 0.1% HCl and concentrated. The residue was re-dissolved in 2:3 Ac₂O-MeOH (5 ml) and stirred overnight, when TLC (17:1 toluene-EtOH) showed disappearance of the starting material. After concentration, the residue was purified on silica gel (97:3 toluene-EtOH) to afford 68 mg of product 25 (84%). [α]_{D}²⁴=+34.06 (c 0.29, CHCl₃).

¹H NMR (CDCl₃, 400 MHz): δ=7.45-7.06 (m, 75H, 15×Ph), 5.81 (d, J_{NH,2}=6.12 Hz, 1H, NH^{B/B′}), 5.66 (d, J_{NH,2}=5.8 Hz, 1H, NH^{B/B′}), 5.36 (m, 2H, NH^{Cbz}, PhCH), 5.08 (d, J_{1,2}=2.4 Hz, 1H, H-1^{D}), 5.04-4.87 (m, 5H, CH₂^{Cbz}, 2×HCH), 4.83-4.57 (m, 10H, 8×HCH), 4.54-4.29 (m, 16H, 16×HCH), 4.27-3.35 (m, 40H, H-1^{C,C′}, 2^{A,B,B′,C,C′,D}, 3^{A,B,B′,C,C′,D}, 4^{A,B,B′,C,C′,D}, 5^{A,B,B′,C,C′,D}, 6^{A,B,B′,C,C′,D}, 1′), 3.17 (m, 1H, H-3′a), 3.08 (m, 1H, H-3′b), 1.70 (s, 3H, CH₃CO), 1.69-1.59 (m, 2H, H-2′), 1.59 (s, 3H, CH₃CO). ¹³C NMR (CDCl₃, 100 MHz): δ=172.39, 172.05, 156.60 (3×CONH), 139.40-126.39 (Ar), 104.50 and 104.06 (C-1^{C,C′}), 100.73 (CHPh), 99.93 and 98.96 (C-1^{B,B′}), 98.18 (C-1^{A}), 97.97 (C-1^{D}), 84.46, 80.32, 79.78, 79.46, 77.97, 77.81, 77.30, 77.20, 76.98, 76.49, 76.31, 75.70, 75.59, 75.12, 75.02, 74.95, 74.90, 74.67, 74.34, 74.04, 73.78, 73.39, 73.25, 73.04, 72.74, 71.77, 70.54, 69.37, 68.90, 68.18, 67.92, 66.45 (CH₂^{Cbz}), 64.42 (C-1′), 60.37, 53.99 (C-2^{B,B′}), 37.72 (C-3′), 29.68 (C-2′), 23.52, 20.46 (2×CH₃CO). ESI HR-MS (C₁₄₉H₁₆₃N₃O₃₃): m/z=([M+H]⁺ found 2523.1301 calcd 2523.1247).

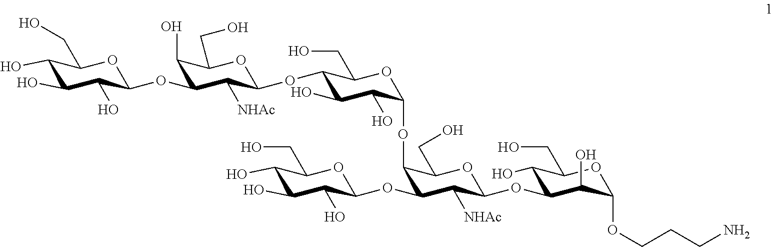

### Aminopropyl-β-D-glucopyranosyl-(1→3)-[β-D-glucopyranosyl-(1→3)-2-acetamido-2-deoxy-β-D-galactopyranosyl-(1→4)-α-D-glucopyranosyl-(1→4)]-2-acetamido-2-deoxy-β-D-galactopyranosyl-(1→3)-α-D-mannopyranoside 1

Compound 25 was deprotected in flow chemistry, using a H-Cube Thales-Nano system.

The protected hexasaccaride (35 mg, 0.014 mmol) was dissolved in 9:1 EtOH/CH₃COOH (30 ml) and hydrogenated over a 10% Pd/C cartridge at 40° C. and pressure=10 bar. The mixture was flown for 1 d, then the solvent was evaporated and the recovered crude material was purified on a C-18 Isolute SPE cartridge, giving 14 mg of the final hexasaccharide 1 (90%). [α]_{D}²⁴=+26.09 (c 0.43, H₂O).

¹H and ¹³C NMR data are reported in Table 2 (¹H and ¹³C-NMR^{a} δ (ppm), recorded at 400 MHz, 298 K, of hexasaccharide 1).

ESI HR-MS (C₄₃H₇₅N₃O₃₁): m/z=([M+H]⁺ found 1130.4412. calc 1130.4463); ([M+Na]⁺ found 1152.4125. calcd 1152.4282).

TABLE 2

| | α-Man | β-GalNAc | β-Glc | β-GalNAc | β-Glc | α-Glc | |
|---|---|---|---|---|---|---|---|
| | (A) | (B) | (C) | (B′) | (C′) | (D) | Linker |
| H-1 | 4.86 | 4.76 | 4.49 | 4.60 | 4.41 | 4.95 | |
| | | J_{1,2} = 8.6 Hz | J_{1,2} = 7.8 Hz | J_{1,2} = 8.6 Hz | J_{1,2} = 7.8 Hz | J_{1,2} = 3.4 Hz | |
| C-1 | 100.5 | 100.3 | 105.3 | 102.3 | 106.0 | 99.6 | |
| H-2 | 4.00 | 4.00 | 3.29 | 4.00 | 3.07 | 3.52 | |
| C-2 | 68.6 | 52.8 | 73.6 | 52.3 | 74.2 | 72.1 | |
| H-3 | 4.00 | 3.90 | 3.45 | 3.90 | 3.46 | 3.97 | |
| C-3 | 79.1 | 79.4 | 76.3 | 80.7 | 76.4 | 72.3 | |
| H-4 | 3.73 | 4.26 | 3.41 | 4.18 | 3.37 | 3.66 | |
| C-4 | 65.9 | 75.5 | 70.3 | 68.6 | 70.6 | 79.8 | |
| H-5 | 3.60 | 3.76 | 3.36 | 3.76 | 3.39 | 4.29 | |
| C-5 | 73.7 | 76.0 | 76.4 | 76.0 | 76.2 | 73.6 | |
| H-6 | 3.76, 3.89 | 3.71, 3.89 | 3.90, 4.18 | 3.75, 3.90 | 3.71, 3.88 | 3.66, 3.82 | |
| C-6 | 61.1 | 61.3 | 65.7 | 61.6 | 61.8 | 60.3 | |
| H-1′ | | | | | | | 3.61, 3.81 |
| C-1′ | | | | | | | 65.7 |
| H-2′ | | | | | | | 1.98 |
| C-2′ | | | | | | | 27.6 |
| H-3′ | | | | | | | 3.10 |
| C-3′ | | | | | | | 38.3 |

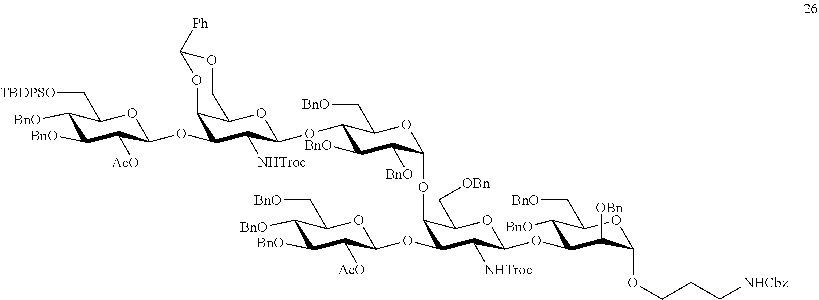

### 3-(Benzyloxycarbonyl)aminopropyl-2-O-acetyl-3,4,6-tri-O-benzyl-β-D-glucopyranosyl-(1→3)-[6-O-tertbutyldiphenylsilyl-2-O-acetyl-3,4-di-O-benzyl-β-D-glucopyranosyl-(1→3)-4,6-O-benzylidene-2-deoxy-2-(2′,2′,2′-trichloroethoxycarbonylamino)-β-D-galactopyranosyl-(1→4)-2,3,6-tri-O-benzyl-α-D-glucopyranosyl-(1→4)]-6-O-benzyl-2-deoxy-2-(2′,2′,2′-trichloroethoxycarbonylamino)-β-D-galactopyranosyl-(1→3)-2,4,6-tri-O-benzyl-α-D-mannopyranoside 26

Donor 4 can be a thioglycoside (i.e. SPh, EtS), sulfoxide, imidate (CF₃CNHPh, CCl₃CNH), alogen (F, Cl, Br, I), phosphinite.

A solution of acceptor 5 (203 mg, 0.11 mmol) and donor 4 (180 mg, 0.16 mmol) was stirred at 0° C. in presence of 4 Å MS, under nitrogen atmosphere. After addition of NIS (39.6 mg, 0.018 mmol) and TfOH (4 μl, 0.05 mmol) the mixture turned immediately red and the reaction mixture was stirred for 6 h at 0° C. Any other promoter could be employed (TMSOTf, NIS-TfOH, BF₃Et₂O) with 70° C.<t<25° C.). When TLC (toluene-EtOH 17:1) showed the reaction was complete, it was quenched with a drop of triethylamine and concentrated. The residue was purified on silica gel (95:5→41:1 cyclohexane-EtOAc) to yield 184 mg of hexasaccharide 26 (62%). [α]_{D}²⁴=+18.4 (c 0.5, CHCl₃).

¹H NMR (CDCl₃, 400 MHz): δ=7.66-7.03 (m, 80H, 16×Ph), 5.89 (d, J_{NH,2}=6.5 Hz, 1H, NH), 5.78 (d, J_{NH,2}=6.0 Hz, 1H, NH), 5.39 (m, 1H, NH^{Cbz}), 5.31 (s, 1H, PhCH), 5.16 (d, J_{1,2}=2.1 Hz, 1H, H-1^{D}), 5.05-4.96 (m, 5H, 2^{C/C′}, CH₂^{Cbz}, incl. 5.03 and 5.00, H-1^{B}, 1^{B}), 4.89 (t, J=8.6 Hz, 1H, H-2^{C/C′}), 4.87-4.38 (m, 25H, 12×CH₂, incl. s, 4.76, H-1^{A}), 4.37-3.62 (m, 40H, 2×CH₂, H-2^{A,B/B′,D}, 3^{A,B,B′,C,C′,D,} 5^{A,B/B′,C,C′,D},6^{A,B,B′,C,C′,D}, 1′, H-1^{C,C′}), 3.30-3.20 (m, 2H, H-2^{B/B′}, 3′a), 3.15 (m, 1H, H-3′b), 2.95 (s, 1H, H-5^{B/B′}), 1.87, 1.92 (2×s, 6H, 2×CH₃CO), 1.73 (m, 2H, H-2′), 1.05 (s, 9H, t-Bu). ¹³C NMR (CDCl₃, 100 MHz): δ=170.11, 169.70 (2×CO), 156.51, 153.69, 153.87 (3×CONH), 139.73-126.16 (Ar, C(CH₃)₃), 102.38, 102.20 (C-1^{C,C′}), 100.13 (CHPh), 99.80, 98.83 (C-1^{B,B′}), 97.97 (C-1^{A}), 97.83 (C-1^{D}), 95.87, 95.54 (2×CCl₃), 83.05, 82.60 (C-3^{B,B′}), 80.76, 80.56, 78.96, 78.14 (C-3^{C,C′}, 4^{C,C′}), 77.24, 76.72, 76.35, 75.96, 75.82, 75.10 (C-3^{A,D}, 4^{A,B,B′,D}, 5^{C,C′}), 74.99, 74.49, 73.97, 73.69, 73.38, 73.18, 73.02, 72.74, 72.42, 71.89, 70.86, 69.55, 69.08, 68.78, 68.57, 68.20, 67.98, 67.95, 66.50, 66.22, 64.25 (C-1′), 63.03, 55.07 (C-2^{B,B′}), 54.56 (C-2^{B/B′}), 37.55 (C-3′), 29.37 (C-2′), 26.81 (t-Bu), 21.10, 20.88 (2×CH₃CO). ESI HR-MS (C₁₆₄H₁₇₇Cl₆N₃O₃₇Si): m/z=([M+Na]⁺ found 3040.9955. calcd 3040.9859).

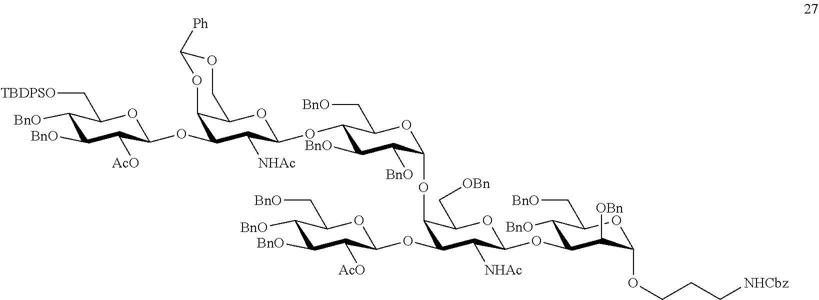

### 3-(Benzyloxycarbonyl)aminopropyl-2-O-acetyl-3,4,6-tri-O-benzyl-β-D-glucopyranosyl-(1→3)-[6-O-tertbutyldiphenylsilyl-2-O-acetyl-3,4-di-O-benzyl-β-O-D-glucopyranosyl-(1→3)-2-acetamido-4,6-O-benzylidene-2-deoxy-)-β-D-galactopyranosyl-(1→4)-2,3,6-tri-O-benzyl-α-D-glucopyranosyl-(1→4)]-2-acetamido-6-O-benzyl-2-deoxy-β-D-galactopyranosyl-(1→3)-2,4,6-tri-O-benzyl-α-D-mannopyranoside 27

The hexasaccharide 26 (280 mg, 0.09 mmol) was dissolved in THF (5 ml) and 2 M NaOH was added. Zn in AcOH or Ac₂O can be used. After refluxing for 2 d (TLC, 15:1 toluene-EtOH), the mixture was neutralized with 0.1% HCl and concentrated. The residue was re-dissolved in 2:3 pyridine-Ac₂O (5 ml) and stirred overnight, when TLC (17:1 toluene-EtOH) showed disappearance of the starting material. After concentration, the residue was purified on silica gel (20:1 toluene-EtOH) to afford 180 mg of product 27 (76%). [α]_{D}²⁴=+47.42 (c 0.6, CHCl₃).

¹H NMR (CDCl₃, 400 MHz): δ=7.72-7.01 (m, 80H, 16×Ph), 5.48-5.35 (m, 3H, PhCH, 2 NH), 5.23-4.88 (m, 7H, NH^{Cbz}, 2^{C,C′}, H-1^{B,B′}, HCH, incl. d, 5.14, J_{1,2}=3.1 Hz, H-1^{D}), 4.88-4.40 (m, 28H, 25×HCH, incl. s, 4.82H-1^{A}, H-1^{C,C′}) 4.38-3.38 (m, 33H, H-2^{A,D}, 3^{A,B,B′,C,C′,D}, 4^{A,B,B′,C,C′,D}, 5^{A,B/B′,C,C′,D}, 6^{A,B,B′,C,C′,D}, 1′), 3.29-3.18 (m, 5H, 2^{B,B′}, 5^{B,B′}, 3′), 1.89-1.71 (m, 14H, H-2′, incl. 4×s, 1.88, 1.83, 1.78, 1.72, 4×CH₃CO), 1.05 (s, 9H, t-Bu). ¹³C NMR (CDCl₃, 100 MHz): δ=171.70, 171.37, 170.20, 169.32, 156.38 (5×CO), 139.85-125.20 (Ar, C(CH₃)₃), 101.80, 101.78 (C-1^{C,C′}), 99.72 (CHPh), 99.81 (C-1^{B,B′}), 97.41 (C-1^{A,D}), 83.07, 82.50 (C-3^{B,B′}), 81.00, 80.76, 79.76, 79.51, 77.82, 75.96, 75.70, 75.23, 75.00, 74.97, 74.73, 74.35, 73.65, 73.44, 72.99, 72.87, 72.46, 72.11, 71.82, 71.60, 70.45, 69.44, 68.57, 67.89, 66.31, 65.77, 64.47 (C-1′), 63.01, 55.43 (C-2^{B/B′}), 53.71 (C-2^{B/B′}), 36.94 (C-3′), 29.25 (C-2′), 26.77 (t-Bu), 24.06, 23.38, 21.10, 20.88 (4×CH₃CO). ESI HR-MS (C₁₆₂H₁₇₉N₃O₃₅Si): m/z=([M+Na]⁺ found 2777.2048. calcd 2777.1986).

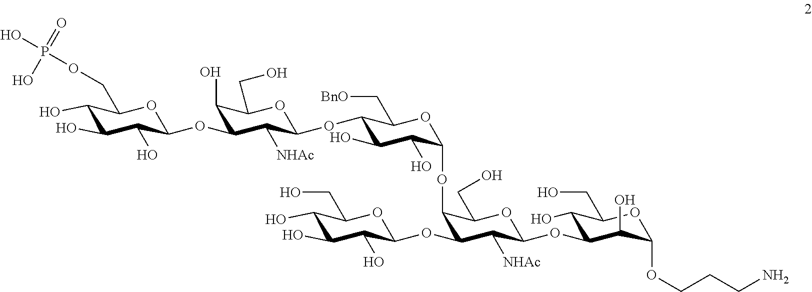

### 3-Aminopropyl-β-D-glucopyranosyl-(1→3)-[6-O-phospho-2-acetamido-2-deoxy-β-D-glucopyranosyl-(1→3)-)-β-D-galactopyranosyl-(1→4)-α-D-glucopyranosyl-(1→4)]-2-acetamido-2-deoxy-β-D-galactopyranosyl-(1→3)-α-D-mannopyranoside 2

To a solution of the silylated hexasaccharide 27 (95 mg, 0.034 mmol) in THF (3 ml) 0.1 M TBAF in THF (1 ml, 0.1 mmol) was added at 0° C. After stirring for 2 h at ambient temperature TLC (17:1 toluene-EtOH) showed complete deprotection. The solvent was evaporated and the residue was purified on silica gel (20:1 toluene-EtOH) to afford 85 mg of product 28 (94%). The product showed disappearance of the tBu signal at ¹H NMR.

ESI HR-MS (C₁₄₆H₁₆₁N₃O₃₅): m/z=([M+Na)]⁺ found 2539.0872; calc 2539.0808).

1H-Tetrazole 0.45 M in acetonitrile (1.8 ml, 0.8 mmol) was added to a solution of the foregoing hexasaccharide 28 (65 mg, 0.026 mmol) and N,N-diethyl-1,5-dihydro-3H-2,4,3-benzodioxaphosphepin-3-amine (19 mg, 0.08 mmol) in CH₂Cl₂ (8 mL). Fmoc phosphonate can be used. After the reaction mixture was stirred at room temperature for 40 min, TLC (17:1 toluene-EtOH) showed formation of a new product. The mixture was cooled to −20° C., then 3-chloroperoxybenzoic acid (m-CPBA) (50 mg, 50-55% wt, 0.11 mmol) or I₂ was added. The reaction mixture was stirred at −20° C. for 20 min (TLC, 17:1 toluene-EtOH), and then quenched by addition of aq NaHCO₃ (3 ml) and diluted with CH₂Cl₂ (10 mL). The solution was washed with aq NaHCO₃ and brine. After work up the organic phase was concentrated, and the residue was purified on silica gel to give 58 mg of phosphorylated product 29 (81%). Introduction of phosphate group was confirmed by ³¹P-NMR and ESI-MS analysis. ³¹P NMR (CDCl₃, 400 MHz): δ=−0.36 ppm. ESI HR-MS (C₁₅₄H₁₆₈N₃O₃₈P): m/z=([M+Na]⁺ found 2721.0991; calcd 2721.0941).

The phosphorylated hexasaccharide 29 was then deprotected in flow chemistry, using a H-Cube Thales-Nano system. Compound 29 (38 mg, 0.014 mmol) was dissolved in MeOH/H₂O 9:1 (10 ml) and hydrogenated for 6 h, over a Pd/C 10% cartridge at ambient temperature and atmospheric pressure. The solvent was then evaporated and the crude material was dissolved in 1:1 MeOH/H₂O (2 ml). A 0.5 M methanolic solution of NaOMe was added until pH=9 and the mixture was left to react at room temperature over night. The mixture was then neutralized with aq 0.1% HCl and evaporated. The crude obtained was desalted using a G10 PD MiniTrap™ GE Healthcare cartridge, giving 19 mg of the final hexasaccharide 2 (99%). ¹H and ¹³C NMR data are reported in Table 2.

[α]_{D}²⁴=+18.68 (c 0.23, H₂O).

ESI HR-MS (C₄₃H₇₆N₃O₃₄P): m/z=([M+H]⁺ found 1210.4080 calcd 1210.4126); ([M+Na]⁺ found 1232.3951 calc 1232.3946).

¹H and ¹³C NMR data are reported in Table 3 (¹H and ¹³C-NMR^{a} δ (ppm), recorded at 400 MHz, 298 K, of hexasaccharide 2).

TABLE 3

| | α-Man | β-GalNAc | β-Glc | β-GalNAc | β-Glc | α-Glc | |
|---|---|---|---|---|---|---|---|
| | (A) | (B) | (C) | (B′) | (C′) | (D) | Linker |
| H-1 | 4.86 | 4.76 | 4.49 | 4.60 | 4.41 | 4.95 | |
| | | J_{1,2} = 8.6 Hz | J_{1,2} = 7.8 Hz | J_{1,2} = 8.6 Hz | J_{1,2} = 7.8 Hz | J_{1,2} = 3.4 Hz | |
| C-1 | 100.6 | 100.5 | 105.6 | 102.5 | 106.0 | 99.6 | |
| H-2 | 4.02 | 4.01 | 3.32 | 4.02 | 3.07 | 3.53 | |
| C-2 | 68.9 | 53.0 | 74.1 | 53.0 | 74.2 | 72.4 | |
| H-3 | 4.02 | 4.00 | 3.49 | 3.90 | 3.45 | 3.97 | |
| C-3 | 79.5 | 79.4 | 76.4 | 80.4 | 76.4 | 72.3 | |
| H-4 | 3.74 | 4.26 | 3.48 | 4.22 | 3.31 | 3.66 | |
| C-4 | 66.2 | 75.6 | 70.6 | 68.4 | 70.6 | 79.8 | |
| H-5 | 3.60 | 3.81 | 3.58 | 3.76 | 3.36 | 4.30 | |
| C-5 | 65.6 | 76.3 | 76.2 | 76.3 | 76.2 | 71.0 | |
| H-6 | 3.69, 3.80 | 3.67, 3.90 | 3.90, 4.21 | 3.76, 3.90 | 3.70, 3.91 | 3.66, 3.82 | |
| C-6 | 61.2 | 61.9 | 65.7 | 61.7 | 63.7 | 60.7 | |
| H-1′ | | | | | | | 3.61, 3.81 |
| C-1′ | | | | | | | 65.7 |
| H-2′ | | | | | | | 1.98 |
| C-2′ | | | | | | | 27.6 |
| H-3′ | | | | | | | 3.10 |
| C-3′ | | | | | | | 38.3 |

### B. Conjugation of Synthetic Saccharides

Synthetic saccharides obtained using the methods outlined above were produced. In particular, deprotected tetrasaccharide was prepared according to scheme 8.

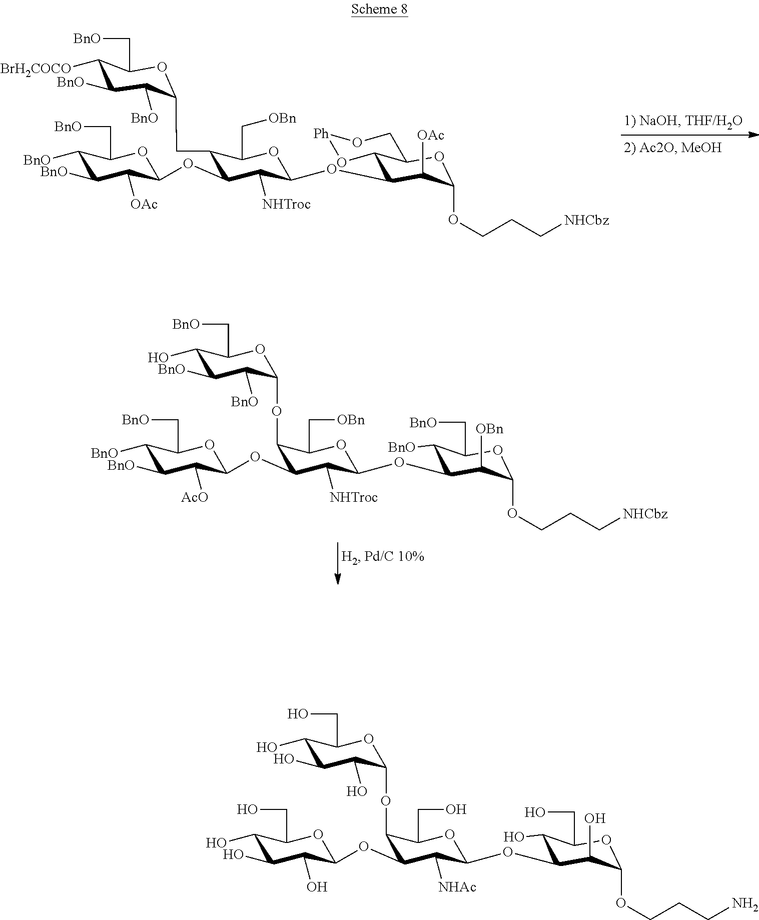

The presence of the synthetic deprotected tetrasaccharide was confirmed using ESI-MS. ¹H NMR confirmed the correct stereochemistry at the four anomeric protons, with residual toluene.

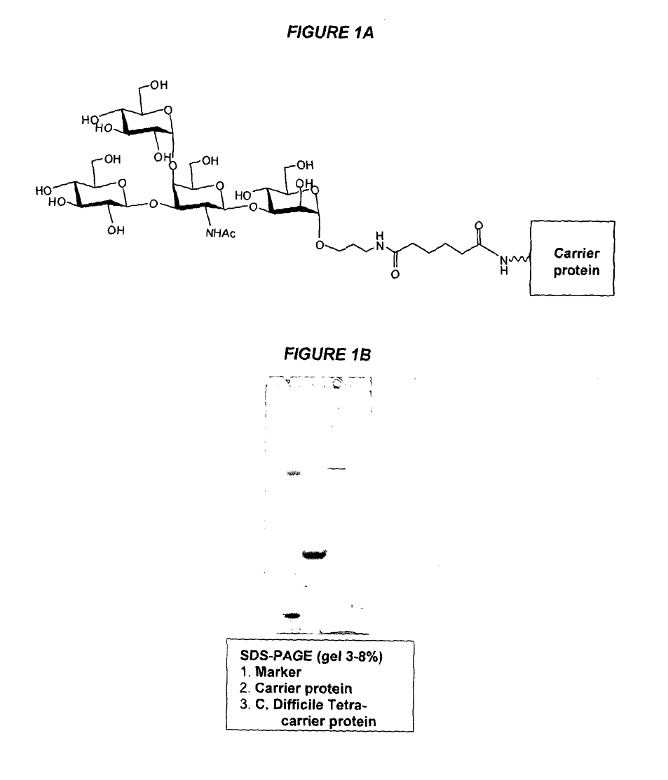

The synthetic tetrasaccharide was conjugated to a carrier protein, yielding the compound shown in FIG. 1a. 8 mg of tetrasaccharide was dissolved in DMSO (500 μl) and reacted with SIDEA (10 eq) and TEA (20 eq) for two hours at room temperature. Precipitation with AcOEt yielded 6.5 mg of crude material. An active ester assay showed that 50% of material had been activated. Carrier protein was then added to the solution at a saccharide:protein ratio of 40:1 (active ester) in NaPi. SDS-PAGE was used to confirm formation of the conjugate (see FIG. 1a for the tetrasaccharide-carrier protein conjugate).

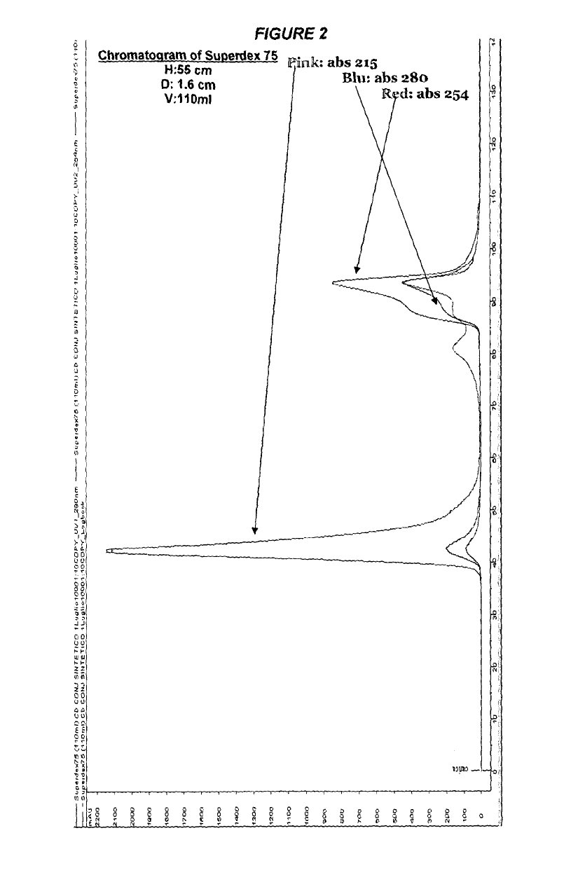

After conjugation, the conjugate was purified using size exclusion chromatography with Superdex 75 resin. The conjugate was detected at 215 nm, 254 nm and 280 nm (FIG. 2). The presence of tetrasaccharide-carrier protein conjugate was confirmed using MALDI spectrometry.

Deprotected synthetic non-phosphorylated hexasaccharide was prepared according to scheme 9.

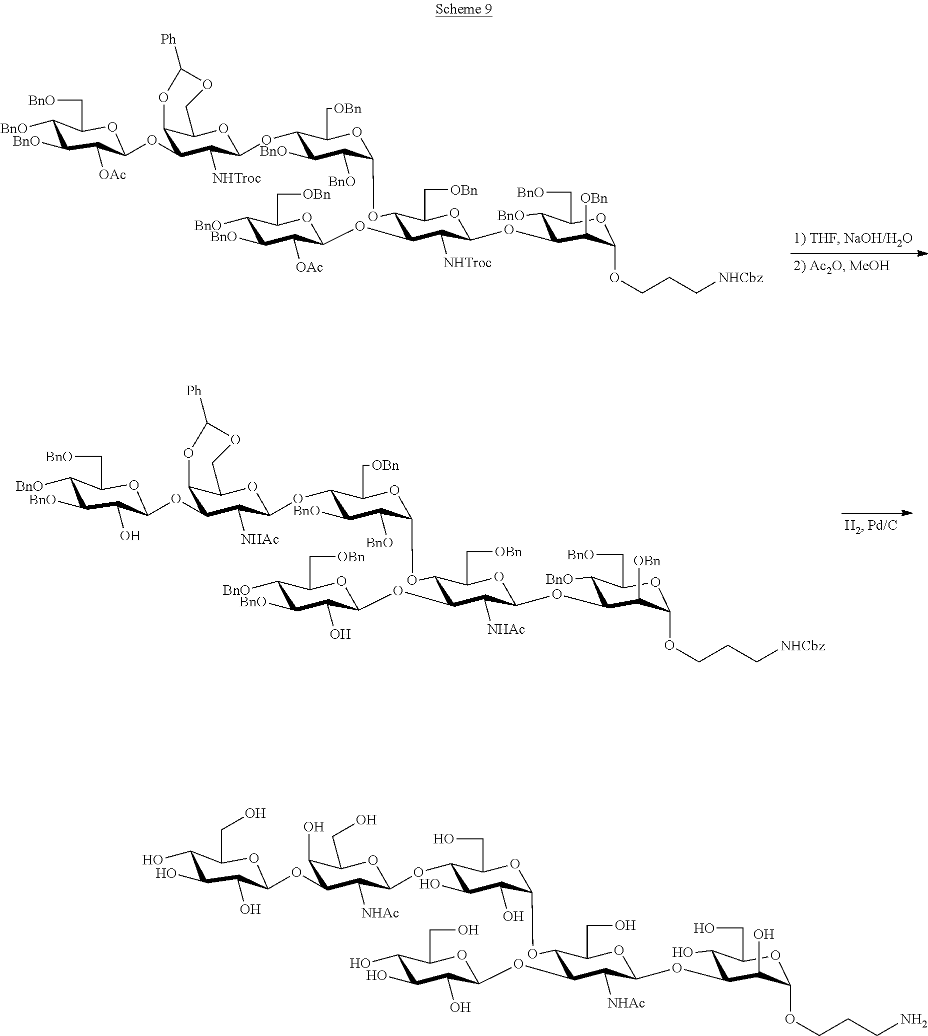

The presence of the non-phosphorylated hexasaccharide was confirmed using ESI-MS. ¹H NMR confirmed the correct stereochemistry at the anomeric protons. The ¹H NMR spectrum (at 50° C.) of this hexaccharide has been compared directly with the ¹H NMR spectrum of the tetrasaccharide (scheme 8).

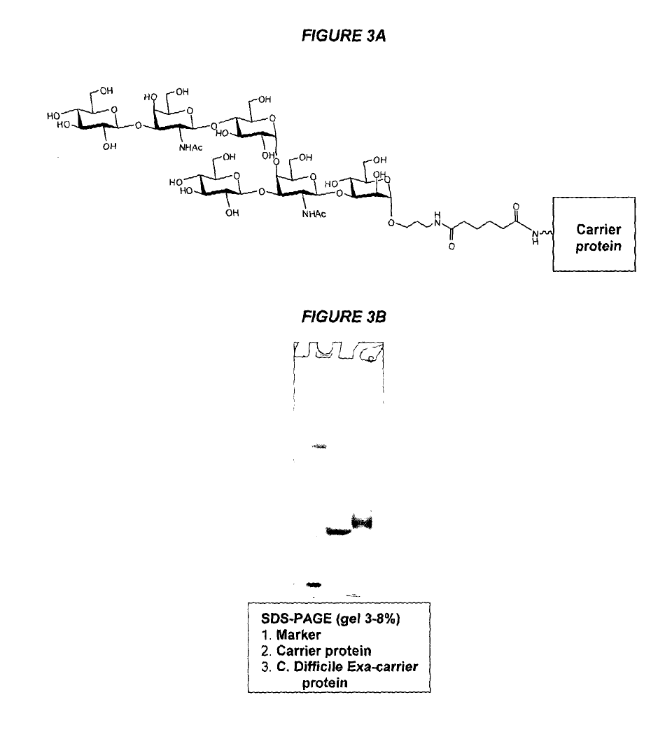

The synthetic hexasaccharide was conjugated to a carrier protein, yielding the compound shown in FIG. 3a. 6 mg of hexasaccharide was dissolved in DMSO (500 μl) and reacted with SIDEA (10 eq) and TEA (20 eq) for two hours at room temperature. Precipitation with AcOEt yielded 1.5 mg of crude material. Carrier protein was then added to the solution at a saccharide:protein ratio of 80:1 in NaPi. SDS-PAGE was used to confirm formation of the conjugate (see FIG. 3b for the hexasaccharide-carrier protein conjugate).

The presence of crude hexasaccharide-carrier protein conjugate was confirmed using MALDI spectrometry.

Deprotected synthetic phosphorylated PS-II hexasaccharide was prepared according to scheme 7 (above).

The presence of the phosphorylated hexasaccharide was confirmed using ESI-MS. ¹H NMR confirmed the correct stereochemistry at the anomeric protons. The ¹H NMR spectrum (at 50° C.) of this hexaccharide has been compared directly with the ¹H NMR spectrum of the PS-II repeating unit (Table 1).

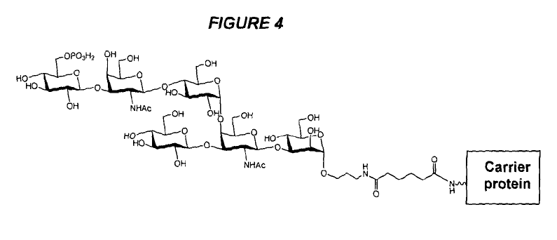

The synthetic phosphorylated PS-II hexasaccharide was conjugated to a carrier protein, yielding the compound shown in FIG. 4. Hexasaccharide was dissolved in DMSO and reacted with SIDEA and TEA. Precipitation with AcOEt yielded crude material. Carrier protein was then added to the solution at a saccharide:protein ratio of 80:1 (Hexa2-CRM₁₉₇) in NaPi (pH 7.0). Also prepared according to the same method were non-phosphorylated hexasaccharide conjugates, Hexa1-CRM₁₉₇ and Hexa1a-CRM₁₉₇. The two non-phosphorylated hexasaccharide conjugates were synthesised using different saccharide:protein and active ester:protein ratios, as shown in Table 4. Also shown in Table 4 are details regarding two non-phosphorylated tetrasaccharide conjugates, Tetra1-CRM₁₉₇ and Tetra1a-CRM₁₉₇.

TABLE 4

| | | Stoichiometry | Stoichiometry | Sugar | | |
|---|---|---|---|---|---|---|
| | | of reaction | of reaction | loading | | |
| | | (mol | (mol active | in | % | % |
| | | CHO/mol | ester/mol | product | conjugation | protein |
| Glycoconjugate | MW | protein) | protein) | (n. chains) | efficiency | recovery |
| Hexa1-CRM₁₉₇ | 75783 | 80 | 40 | 14.1 | 34 | 73 |
| (4) | | | | | | |
| Hexa1a-CRM₁₉₇ | 70972 | 60 | 30 | 9.8 | 33 | 76 |
| (5) | | | | | | |
| Hexa2-CRM₁₉₇ | 71927 | 80 | 40 | 10.5 | 26 | 76 |
| (6) | | | | | | |
| Tetra1-CRM₁₉₇ | 71979 | — | 40 | 15.5 | 39 | 88 |
| (2) | | | | | | |
| Tetra1a-CRM₁₉₇ | 67074 | — | 30 | 9.8 | 33 | 100 |
| (3) | | | | | | |

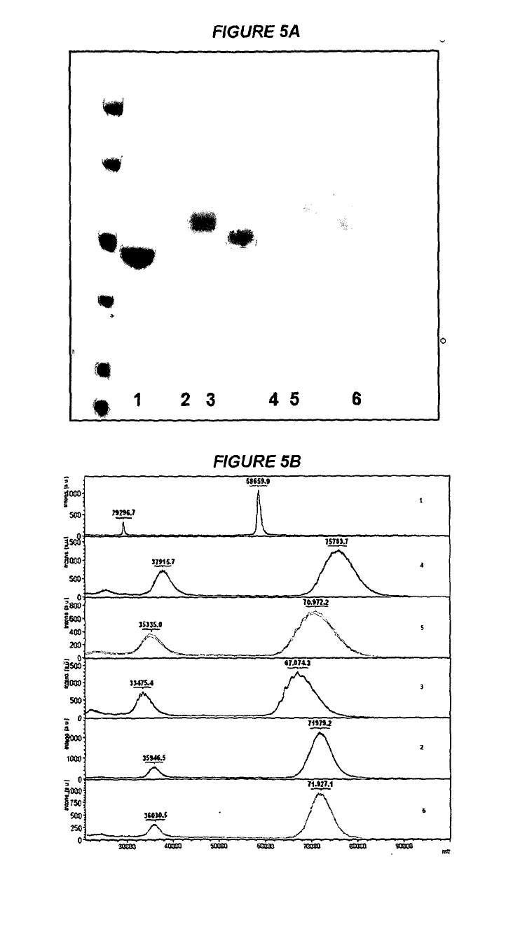

SDS-PAGE was used to confirm formation of the phosphorylated PS-II hexasaccharide-carrier protein conjugate, Hexa2-CRM₁₉₇ (6), the two non-phosphorylated PS-II hexasaccharide-carrier protein conjugates, Hexa1-CRM₁₉₇ (4) and Hexa1a-CRM₁₉₇ (5), and the two non-phosphorylated PS-II tetrasaccharide-carrier protein conjugates, Tetra1-CRM₁₉₇ (2) and Tetra1a-CRM₁₉₇ (3) (FIG. 5a). The presence of these hexasaccharide-carrier protein conjugates was confirmed using MALDI-TOF spectrometry (FIG. 5b).

C. Purification of C. Difficile PS-II Saccharides from C. Difficile Bacterial Cells

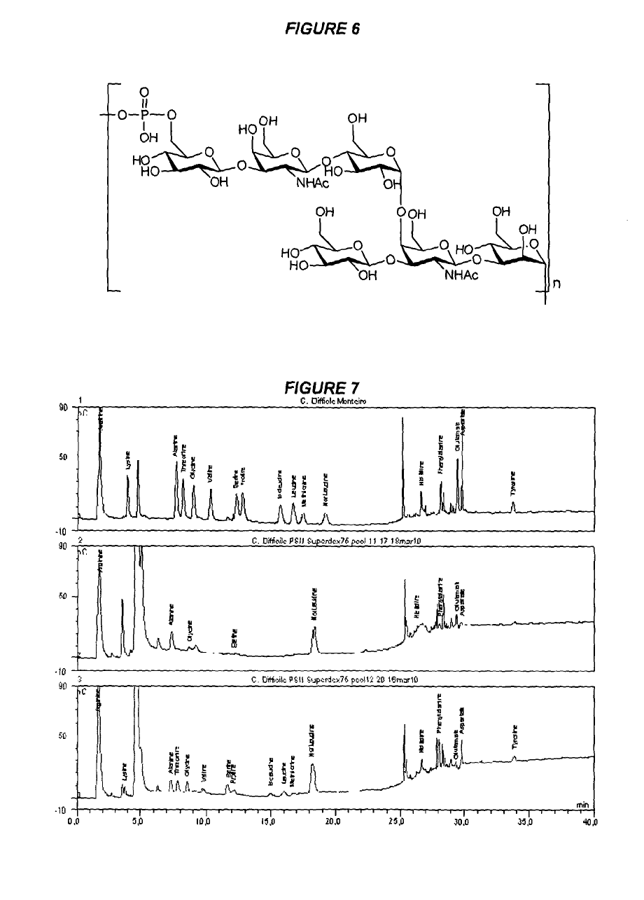

The structure of the C. difficile cell-surface saccharide (PS-II) is shown in FIG. 6. A number of strains able to produce such a PS-II saccharide were tested, including M68, M120, 630, Nt2023 and Stoke-Mandeville. Stoke-Mandeville strain was selected as the best producer and was used in the processes of the present invention. To detect the presence of PS-II saccharide, cells of 40 different clinical isolates recovered from bacterial growths were inactivated by 1% (v/v) formaldehyde treatment and then washed three times with PBS in deuterium oxide (D₂O—Sigma-Aldrich). Approximately 50 μl of compact pellet was inserted into a Kel-F disposable insert and then in 4 mm MAS ZrO2 rotor (Bruker). Proton HR-MAS NMR experiments were recorded by a Bruker Avance III 400 MHz spectrometer using a Bruker 4 mm HR-MAS probe. The TOPSPIN™ software package (Bruker) was used for data acquisition and processing. The transmitter was set at the HDO frequency, which was also used as reference signal (4.79 ppm). The spectra were recorded at a 4500 Hz spin rate and 25° C. The ¹H spectra were acquired with a diffusion filter pulse sequence with gradient pulses (diffusion filter 95%), to remove the low-molecular-mass species free in solution, and a Carr-Purcell-Meiboom-Gill (CPMG) pulse sequence [90-(t-180-t)n-acquisition] as T₂ filter (76.8 ms), to remove the broad signals of larger molecular species.

The saccharides produced using the Stoke-Mandeville strain of C. difficile followed by acetic acid inactivation were purified according to scheme 10.

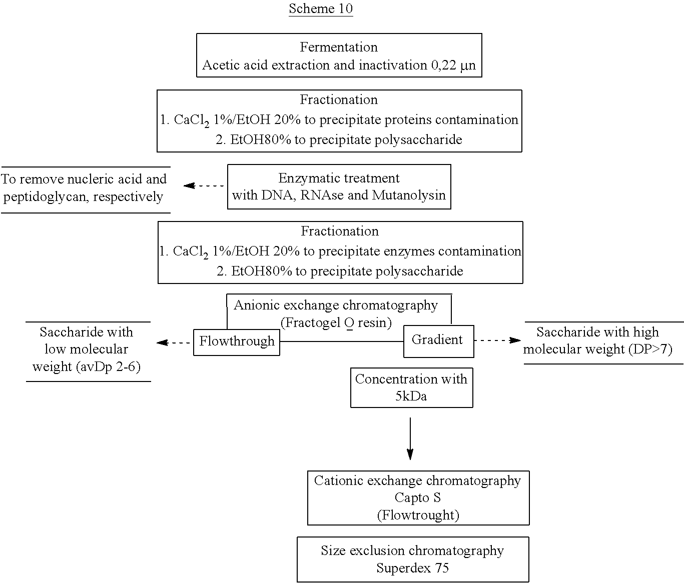

TABLE 5

| Steps | Conditions | Rationale |
|---|---|---|
| Precipitation CaCl₂ | 1. CaCl₂ 1% and EtOH | 1. Removal of protein |
| 1%-EtOH | 20% | contaminants |
| | 2. CaCl₂ 1% and EtOH | 2. Precipitation of PS-II |
| | 80% | (The first step allows the precipitation |
| | | of the proteins and the subsequent |
| | | increase of EtOH concentration allows |
| | | the PS-II precipitation) |
| Enzymatic treatment | 1. DNase and RNase | 1. Removal of nucleic acid |
| | 2. Mutanolysin | Removal of peptidoglycan |
| | | (mutanolysin, an N-acetylmuramidase, |
| | | is a muralytic enzyme that cleaves β- |
| | | N-acetylmuramyl-(1,4)-N- |
| | | acetylglucosamine linkage of |
| | | peptidoglycan) |
| Precipitation CaCl₂ | 1. CaCl₂ 1% and EtOH | 1. Removal of enzymes |
| 1%-EtOH | 20% | contaminants |
| | 2. CaCl₂ 1% and EtOH | 2. Precipitation of PS-II |
| | 80% | (The first step allows the precipitation |
| | | of the enzymes and the subsequent |
| | | increase of EtOH concentration allows |
| | | the PS-II precipitation) |
| Chromatography (1) | Anionic exchange | Selection of PS-II molecular weight: |
| | chromatography (AEC), | low molecular weight (DP 2-6) |
| | Fractogel Q resin (Merck) | recovered in the flowthrough and high |
| | | molecular weight (DP >7) in the |
| | | gradient |
| Tangential Flow | TFF with membrane 5 kDa-cut | Concentration and buffer exchange of |
| Filtration (TFF) | off | the PS-II recovered on the AEC |
| | | gradient |
| Chromatography (2) | Cationic exchange | Removal of positive charged |
| | chromatography (CEC), | contaminants. PS-II recovered in the |
| | CaptoS resin (G&E | flowthrough |
| | healthcare) | |
| Chromatography (3) | Size exclusion | Removal of low molecular weight |
| | chromatography (SEC), | contaminants |
| | Superdex 75 resin (G&E | |
| | healthcare) | |

Following purification using the processes described above, the saccharides were characterized. PS-II saccharide content was estimated using a phenol-sulfuric acid assay [223].

A number of assays were performed to investigate the levels of nucleic acid, amino acid, protein and peptidoglycan contaminants in the purified PS-II saccharides. The level of nucleic acid contaminants were measured by absorption at 260 nm in a spectrophotomer. Total saccharide in the conjugate was determined by HPAEC-PAD analysis and protein content by MicroBCA assay and Bradford analysis. MicroBCA analysis suggested the presence of 18-27 or 10-20% (weight/volume) protein in the polypeptide samples purified according to the present invention, whereas very little protein content (<1% w/v) was detected using Bradford analysis. It appears that the MicroBCA assay overestimates the protein content relative to amino acid analysis using HPAEC-PAD, which obtained a protein concentration in the range of only 1-3.5% w/v. Investigations carried out by the inventors have suggested that the MicroBCA assay was influenced by the reducing group of PS-II saccharide [224]. The mannose group (i.e. the reducing sugar of the repeating unit) is thought to result in levels of interference in the MicroBCA assay of 13-15%. Accordingly, the inventors have attributed the overestimation in protein content measured in this assay to interference by the mannose reducing sugar. Mass spectrometry studies are expected to confirm this.

Amino acid analysis was carried out using HPAEC-PAD. Amino acid analysis consisted of hydrolysis in vacuo with 6M hydrochloric acid for 24 h at 112° C. in order to yield free amino acids from residual protein and peptidoglycan contamination followed by chromatographic analysis using HPAEC-PAD using an AminoPac™ PA1 column and gradient elution in sodium acetate/NaOH. The quantification was performed using a non-hydrolyzed 17 amino acid standard solution in the range 2.5-50 μM (see FIG. 7).

The results of the protein analysis of the PS-II saccharides of the invention using MicroBCA assays HPAEC-PAD compared with the PS-II saccharides according to Monteiro et al. are shown in Table 6.

TABLE 6

| | | Protein μg/mL | | Protein % | |
|---|---|---|---|---|---|
| | Saccharide μg/mL | micro | | micro | |
| Sample | (Phenol/H₂SO₄) | BCA | AAA | BCA | AAA |
| A | 450 | 478 | 356 | 106.2 | 79.1 |
| B | 930 | 183 | 9 | 19.7 | 1.0 |
| C | 210 | 57 | 7 | 27.0 | 3.5 |
| D | 210 | 57 | 8 | 27.0 | 3.9 |

Structural identity and degree of polymerization of PS-II saccharide was verified by NMR analysis. Samples were dissolved in deuterium oxide (D₂O, 99.9%). ¹H and ³¹P NMR experiments were recorded at 50° C. on a Bruker 400 MHz spectrometer, using a 5-mm broadband probe (Bruker). The TOPSPIN™ software package (Bruker) was used for data acquisition and processing. The transmitter was set at the HDO frequency, which was also used as reference signal (4.79 ppm). ID proton NMR spectra were collected using a standard one-pulse experiment.

### D. Conjugation of Purified Native Saccharides

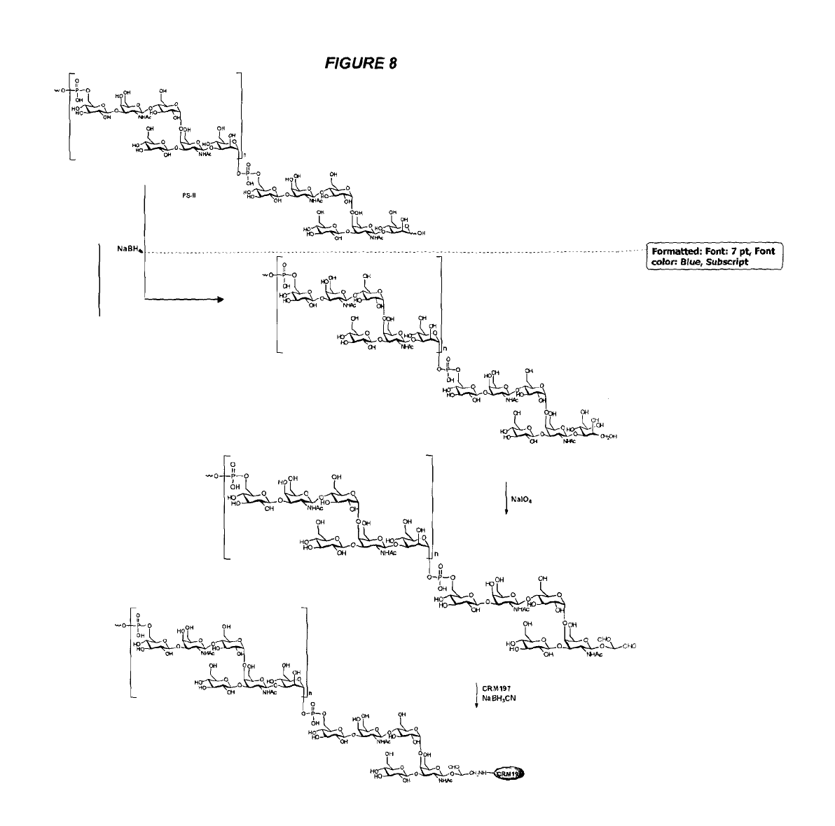

Purified C. difficile PS-II saccharides obtained from the processes in section C above were conjugated to CRM₁₉₇. In light of the structure of C. difficile PS-II saccharides, the inventors postulated that the mannose group acts as a reducing group (since this sugar is involved in an anomeric phosphodiester linkage which is weaker than the other glycosidic bonds and was therefore expected to hydrolyse, leaving the phosphate group on the non-reducing side of the molecule). This has been confirmed by means of Heteronuclear Multiple Bond Correlation analysis (HMBC—¹H and ³¹P). The conjugation strategy was developed accordingly, based on the chemical modification of the mannose located at the reducing end of the saccharide. There are three keys steps to the conjugation process, as outlined in FIG. 8.

In the first step, 1.2 mg/ml of saccharide was reacted with 50 mM NaBH₄ in 10 mM NaPi (pH 9.0) at room temperature for 2 h, followed by purification by gel-filtration chromatography (G25). 1 mL of resin was used for 0.5-0.7 mg of PS-II saccharide.

The next step was oxidation of the saccharide with 4 mM NaIO₄ (15 mol equivalents wrt PS-II) in 10 mM NaPi (pH 7.2) at room temperature for 2 h, in the dark, followed by purification by gel-filtration chromatography (G25).

Finally, the oxidised saccharide was dissolved in a 200 mM NaPi, 1M NaCl (pH 8.0 buffer at a concentration of 10 mg/mL). CRM197 was added to the solution at a saccharide:protein ratio of 4:1 (weight/weight) and NaBH₃CN was added at a sacchaaride:NaBCNH₃ ratio of 2:1 (weight/weight). The solution was kept at 37° C. for 48-72 h.

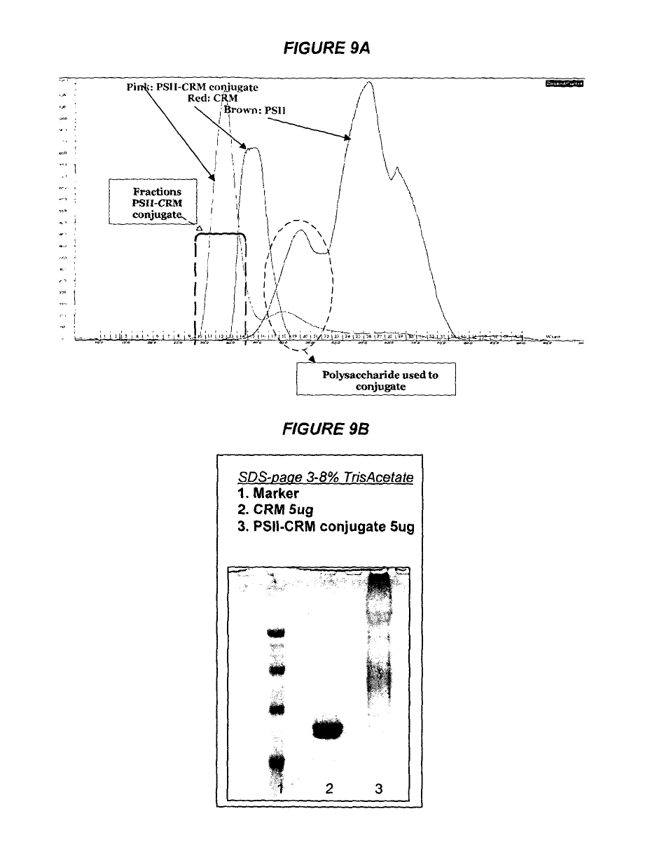

After conjugation, the conjugate was purified by gel-filtration chromatography using Superdex 75 resin, as shown in FIG. 9a.

SDS-PAGE was used to confirm formation of the conjugate (FIG. 9b), using 3-8% tris-acetate gel. Samples of conjugate were mixed with 0.5 M dithiothreitol and NuPAGE LDS sample buffer, and were heated at 100° C. for 1 minute. The gel containing loaded samples was electrophoresed at 45 mA in NuPAGE Tris-Acetate SDS running buffer, and stained with Simply Blue Safe Stain (Invitrogen).

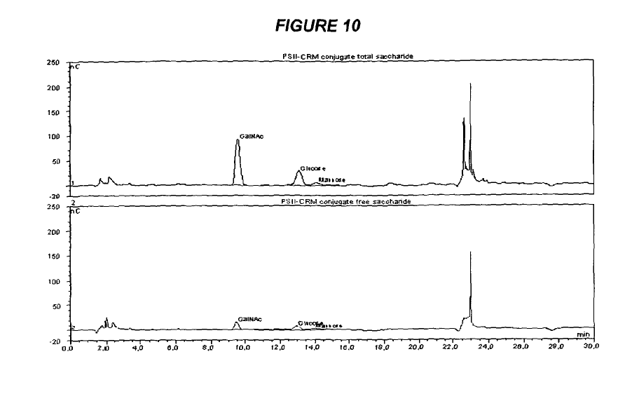

Total saccharide in the conjugate was determined by HPAEC-PAD analysis. Briefly, this consisted of hydrolysis in vacuo with 4M hydrochloric acid for 3 h at 100° C. in order to yield free amino acids from residual protein and peptidoglycan contamination followed by chromatographic analysis using HPAEC-PAD using a CarboPac™ PA1 column and isocratic elution in 18 mM NaOH. The quantification was performed using a calibration curve of GalNAc, Glc and Man in the range 0.5-8.0 μM (see FIG. 10). Free saccharide separation was performed with SPE C4 column. The results of the saccharide quantification analysis are summarized in Table 7. Table 7 also shows average degree of polymerization data for a number of batches of PS-II cell wall saccharides purified using the processes of the invention.

TABLE 7

| | Average degree of | | | | | Free |
|---|---|---|---|---|---|---|
| | polymerisation | Saccharide | Protein | Sacc/Prot | Sacc/Prot | saccharide |
| Conjugate | of saccharide | (μg/mL) | (μg/mL) | (w/w) | (mol/mol) | (%) |
| A | 8 | 50.55 | 165 | 0.31 | 2.07 | — |
| B | 21 | 33.08 | 137 | 0.24 | 0.61 | 11.2 |
| C | 10.7 | 27.01 | 104 | 0.26 | 1.31 | nd |
| D | 8-10.7 | 41.37 | 123 | 0.34 | nd | — |

### E. Immunization Studies Using PS-II-Protein Conjugates

The immunogenicity of various antigens was tested in mice as outlined below.

### First Study in Mice

Groups of CDI mice were immunised by intraperitoneal injection with a 2.5 μg dose of antigen in an injection volume of 200 μl with MF59 and AlumOH as adjuvants. Injections were carried out at 0, 21 and 35 days, with bleeding performed at 0, 34 and 49 days. Immunisations were carried out in groups of eight mice with the following antigens: (i) PBS and (ii) PS-II-CRM₁₉₇ (see summary in Table 8).

TABLE 8

| Antigen Name | Antigen Dose | Imm | VPA | Route |
|---|---|---|---|---|
| PBS | — | 1-2-3 | 200 μL | IP |
| PS-II-CRM₁₉₇ | 2.5 μg | 1-2-3 | 200 μL | IP |
| conjugate | | | | |

### Second Study in Mice

Groups of CDI mice were immunised by intraperitoneal injection with a 2.5 μg dose of antigen in an injection volume of 200 μl with MF59 and AlumOH as adjuvants. Injections were carried out at 0, 21 and 35 days, with bleeding performed at 0, 34 and 49 days. Immunisations were carried out in groups of eight mice with the following antigens: (i) PBS or (ii) PS-II-CRM₁₉₇ (see summary in Table 9).

TABLE 9

| Antigen Name | Antigen Dose | Imm | VPA | Route |
|---|---|---|---|---|
| PBS | — | 1-2-3 | 200 μL | IP |
| PS-II-CRM₁₉₇ | 2.5 μg | 1-2-3 | 200 μL | IP |
| conjugate | | | | |

### Third Study in Mice

Groups of CDI mice were immunised by intraperitoneal injection with a 2.5 μg dose of antigen in an injection volume of 200 μl with MF59 as adjuvant. Injections were carried out at 0, 21 and 35 days, with bleeding performed at 0, 34 and 49 days. Immunisations were carried out in groups of eight mice with the following antigens: (i) PBS and (ii) PS-II-CRM₁₉₇ (see summary in Table 10).

TABLE 10

| Antigen Name | Antigen Dose | Imm | VPA | Route |
|---|---|---|---|---|
| PBS | — | 1-2-3 | 200 μL | IP |
| PS-II-CRM₁₉₇ | 2.5 μg | 1-2-3 | 200 μL | IP |
| conjugate | | | | |

### Fourth Study in Mice

Groups of BALB/c mice were immunised by intraperitoneal injection with a 2.5 μg dose of antigen in an injection volume of 200 μl with MF59 as adjuvant. Injections were carried out at 1, 21 and 35 days, with bleeding performed at 0, 34 and 49 days. Immunisations were carried out in groups of eight mice with the following antigens: (i) PBS+MF59, (ii) PS-II-CRM₁₉₇ conjugate, (iii) Hexa1a-CRM₁₉₇ (see Table 4, above) and (iv) Hexa2-CRM₁₉₇ (see Table 4, above), as summarised in Table 11.

TABLE 11

| Antigen Name | Antigen Dose | Imm | VPA | Route |
|---|---|---|---|---|
| PBS | — | 1-2-3 | 200 μL | IP |
| PS-II-CRM₁₉₇ | 2.5 μg | 1-2-3 | 200 μL | IP |
| conjugate | | | | |
| Hexa1a-CRM₁₉₇ | 2.5 μg | 1-2-3 | 200 μL | IP |
| Hexa2-CRM₁₉₇ | 2.5 μg | 1-2-3 | 200 μL | IP |

Analysis of Results from Mice Studies

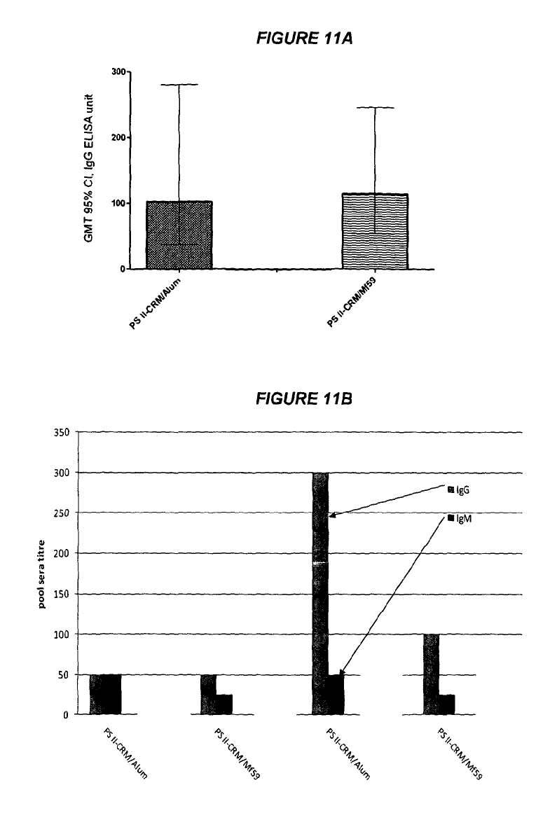

Mice sera were initially tested for the presence of anti-PS-II antibodies using an Enzyme-linked immunosorbent assay (ELISA) procedure based on direct coating of PS-II on the plates. The results of the assay showed that the conjugate was able to induce low titers of anti-PS-II IgG (FIGS. 11a and 11b) and anti-PS-II IgM (FIG. 11b) in some of the immunized mice.

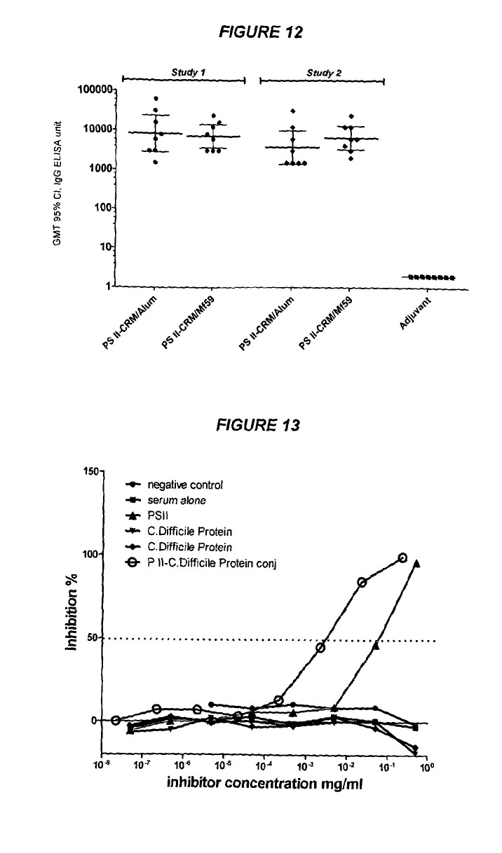

The inventors were concerned that the coating procedure for that anti-PS-II ELISA was neither efficient nor consistent. In particular, they hypothesized that direct coating of saccharides on plastic plates may always be inefficient. Thus, they coated the ELISA plates with PS-II conjugated to recombinant protein from C. difficile. Sera of mice immunized with PS-II-CRM₁₉₇ conjugate were then tested on these plates. Adopting this procedure, the inventors found a very high anti-PS-II IgG response in all the immunized mice, both with AlumOH and MF59 as an adjuvant (FIG. 12). Statistical analysis on the median distribution of these data show that the difference between the use of AlumOH and MF59 as adjuvant is not significant.

Further immunogenic studies were carried out with ELISA plates coated with PS-II-HSA with PS-II conjugated to CRM₁₉₇, and synthetic C. difficile PS-II cell wall tetrasaccharide and hexasaccharide (phosphorylated and non-phosphorylated). These showed that synthetic phosphorylated C. difficile PS-II cell wall hexasaccharide is immunogenic.

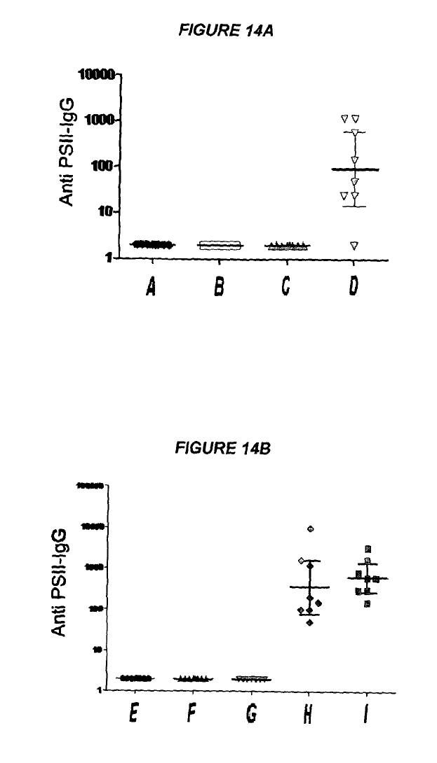

The specificity of the immunological response was assessed by competitive ELISA on sera of mice immunized with PS-II-CRM197 conjugate against PS-II conjugated to recombinant protein from C. difficile. Purified PS-II and PS-II conjugated to recombinant protein were found to inhibit the reaction between the immune serum obtained from immunization with PS-II-CRM₁₉₇ conjugate and the PS-II recombinant protein conjugate coated on the plates, as shown in FIG. 13. PS-II was able to inhibit up to 100%, with IC₅₀=0.068 mg/mL. PS-II-recombinant protein conjugate was also able to inhibit up to 100%, with IC₅₀=0.003 mg/mL. C. difficile recombinant protein alone did not inhibit the ELISA signal, demonstrating that the antibodies detected are all directed against the polysaccharide structure.

The results of further immunogenic studies on the synthetic PS-II hexasaccharide conjugates using ELISA plates coated with PS-II-HSA are shown in FIGS. 14a and b. Synthetic phosphorylated PS-II hexasaccharide conjugate (Hexa2-CRM₁₉₇, (D)+(H)) and purified PS-II-CRM₁₉₇ (I) exhibited immunogenicity. These data suggest that the phosphate group is included in the PS-II minimal epitope, and suggest that phosphorylated PS-II hexasaccharide could be an antigen for the development of a synthetic vaccine against C. difficile.

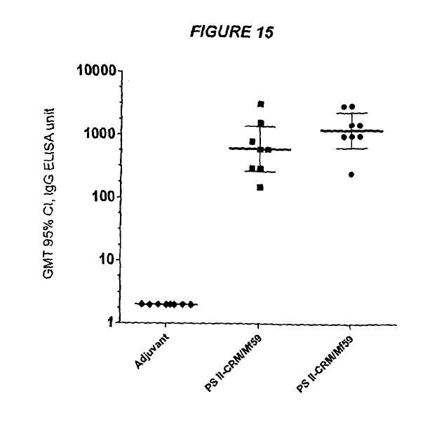

FIG. 15 shows the IgG results of studies using sera from BALB/c mice on PS-II-HSA coated plates, confirming that PS-II-CRM₁₉₇ is able to induce high anti PS-II antibodies (titer ca. 1000 ELISA units).

To carry out the ELISA assays described above, specific antibodies titers were determined 2 weeks after the second and the third immunization. For that purpose 96-well Maxisorp plates were coated with 100 μL/well of 8 μg/mL PS-II in PBS pH 8.2 or 2 μg/mL PS-II conjugated to recombinant protein in PBS pH 7.2 or 2 μg/mL PS-II-HSA conjugate in PBS pH 7.2. Plates were incubated overnight at 4° C., then washed three times with TPBS (0.05% Tween 20 in PBS, pH 7.4) and blocked with 100 μL/well of 3% BSA for 1 hour at 37° C. Each incubation step was followed by triple TPBS wash. Serum samples were initially diluted 1:100-1:1000 in TPBS, transferred into coated-blocked plates (200 μL) and serially two-fold diluted followed by 2 hours incubation at 37° C. 100 μL/well of 1:2000-1:5000 diluted alkaline phosphatase-conjugated goat anti-mouse IgG or IgM was then added and left for 1 hour at 37° C. Visualization of bound alkaline phosphatase was performed by adding 100 μL/well of 1 mg/mL para-nitrophenyl-phosphate (pNPP) disodium hexahydrate in 0.5 M diethanolamine buffer pH 9.6. After 30 minutes of development at room temperature, plates were read at 405 nm with a microplate spectrophotometer. Antibody titres were expressed as the reciprocal of sera dilution corresponding to a cut off OD=1.0. Each group of immunization was represented as the geometrical mean (GMT) of the single mouse titers.

### Hamster Challenge Model

The experiment consisted of 10 animals: 6 animals immunised with the conjugate using MF59 as adjuvant; 2 animals immunised with adjuvant alone; and 2 animals as environmental controls. Groups of hamsters were immunised by intraperitoneal injection with a 15 μg dose (based on the amount of saccharide) of conjugate in an injection volume of 200 μl with MF59 as adjuvant. Injections were carried out at 0, 14, 28 and 42 days, Animals were treated with clindamycin and approx. 18 h after received ˜250 spores each (from strain B1).

The hamster challenge model outlined above is expected to provide further evidence of the protective activity of these antibodies.

### Confocal Immunofluorescence Microscopy

To verify the presence of PS-II on the bacterial surface, sera raised against purified native PS-II and synthetic saccharide units conjugated with CRM₁₉₇ or another carrier protein were used for confocal microscopy studies.

Strains were grown overnight in BHI. Bacteria were recovered by centrifugation, washed with PBS, fixed with 1.5% PFA for 20 minutes at room temperature and spotted on chamber slides coated with polylysine. Bacteria were blocked with 2% BSA for 15 minutes and incubated with sera diluted 1/250 in 2% BSA for 1 hour at room temperature. Bacteria were then stained with goat anti-mouse Alexa Fluor 568 conjugated antibodies (Molecular Probes) for 30 minutes at room temperature. Gold antifade reagent with DAPI (Molecular Probes) was used to mount cover slips.

The inventors found that sera PS-II-CRM₁₉₇ (purified native), Hexa2-CRM₁₉₇ (phosphorylated) and Hexa2-carrier protein (phosphorylated) were able to recognize PS-II structures on the surface of the SM strain, whereas sera Tetra1-CRM₁₉₇ (non-phosphorylated) and Hexa1a-CRM₁₉₇ (non-phosphorylated) gave no or minor staining of the bacteria. In contrast, none of the sera tested were able to recognize PS-II structures on the surface of the 630 strain.

It will be understood that the invention has been described by way of example only and modifications may be made whilst remaining within the scope and spirit of the invention.

### REFERENCES

[1] Koo (2010) Expert Opin. Invest. Drugs, 825.
[2] Knoop et al. (1993) Clin. Microbiol. Rev., 6, 251.
[3] Freeman et al. (2010) Clin. Microbiol. Rev., 23, 529.
[4] J-Pépin et al. (2004) JAMC, 171, 466.
[5] Loo et al. (2005) N. Engl. J. Med., 353, 2442.
[6] Redelings et al. (2007) Emerg. Infect. Dis., 13, 1417.
[7] Gerding et al. (2008) Clin. Infect. Dis., 46, S32.
[8] Sun et al. (2010) Toxins, 2, 1848.
[9] Rupnik (2009) Nat. Rev. Microbiol., 7, 526.
[10] Ganeshapillai et al. (2008) Carbohydr. Res., 343, 703.
[11] Greene et al. (2006) “Protective groups in organic chemistry” John Wiley and Sons, 4^{th} Edition, 2006.
[12] U.S. Pat. No. 4,882,317.
[13] U.S. Pat. No. 4,695,624.
[14] Mol. Immunol., 1985, 22, 907-919.
[15] EP-A-0208375.
[16] WO00/10599.
[17] Gever et al. (1979) Med. Microbiol. Immunol. 165, 171-288.
[18] U.S. Pat. No. 4,057,685.
[19] U.S. Pat. Nos. 4,673,574; 4,761,283; 4,808,700.
[20] U.S. Pat. No. 4,459,286.
[21] U.S. Pat. No. 4,965,338
[22] U.S. Pat. No. 4,663,160.
[23] U.S. Pat. No. 4,761,283
[24] U.S. Pat. No. 4,356,170
[25] Bardotti et al. (2008) Vaccine, 26, 2284.
[26] Dubois et al. (1951) Nature, 168, 167.
[27] WO02/058737.
[28] Ravenscroft et al. (1999) Vaccine, 17, 2802.
[29] Broker et al. (2009) Vaccine, 27, 5574.
[30] Ramsay et al. (2001) Lancet, 357, 195.
[31] Lindberg (1999) Vaccine, 17 Suppl 2, S28-36.
[32] Buttery & Moxon (2000) J. R. Coll. Physicians. Lond., 34, 163.
[33] Ahmad & Chapnick (1999) Infect. Dis. Clin. North. Am., 13, 113.
[34] Goldblatt (1998) J. Med. Microbiol., 47, 563.
[35] European patent 0477508.
[36] U.S. Pat. No. 5,306,492,
[37] WO98/42721.
[38] Dick et al. (1989) Conjugate Vaccines (eds. Cruse et al.) Karger, Basel, 10, 48.
[39] Hermanson (1996) Bioconjugae Techniques, Academic Press, San Diego.
[40] Research Disclosure, 453077 (January 2002)
[41] EP-A-0372501.
[42] EP-A-0378881.
[43] EP-A-0427347.
[44] WO93/17712
[45] WO94/03208.
[46] WO98/58668.
[47] EP-A-0471177.
[48] WO91/01146
[49] Falugi et al. (2001) Eur. J. Immunol., 31, 3816.
[50] Baraldo et al. (2004) Infect. Immun., 72, 4884.
[5] EP-A-0594610.
[52] Ruan et al. (1990) J. Immunol., 145, 3379.
[53] WO00/56360.
[54] WO02/091998.
[55] Kuo et al. (1995) Infect. Immun., 63, 2706.
[56] Michon et al. (1998) Vaccine, 16, 1732.
[57] WO01/72337
[58] WO2004/041157.
[59] WO02/34771.
[60] WO99/42130
[61] WO96/40242.
[62] Lei et al. (2000) Dev. Biol. (Basel), 103, 259.
[63] WO00/38711; U.S. Pat. No. 6,146,902.
[64] WO99/24578.
[65] WO99/36544.
[66] WO99/57280.
[67] WO00/22430.
[68] Tettelin et al. (2000) Science, 287, 1809.
[69] WO96/29412.
[70] Pizza et al. (2000) Science, 287, 1816.
[71] WO01/52885.
[72] Bjune et al. (1991) Lancet, 338, 1093.
[73] Fukasawa et al. (1999) Vaccine, 17, 2951.
[74] Rosenqvist et al. (1998) Dev. Biol. Stand., 92, 323.
[75] Costantino et al. (1992) Vaccine, 10, 691.
[76] WO03/007985.
[77] Watson (2000) Pediatr. Infect. Dis. J., 19, 331.
[78] Rubin (2000) Pediatr. Clin. North. Am., 47, 269.
[79] Jedrzejas (2001) Microbiol. Mol. Biol. Rev., 65, 187.
[80] Bell (2000) Pediatr. Infect. Dis. J., 19, 1187.
[81] Iwarson (1995) APMIS, 103, 321.
[82] Gerlich et al. (1990) Vaccine, 8, S63-68 & 79-80.
[83] Hsu et al. (1999) Clin. Liver. Dis., 3, 901.
[84] Gustafsson et al. (1996) N. Engl. J. Med., 334, 349.
[85] Rappuoli et al. (1991) TIBTECH, 9, 232.
[86] Vaccines (2004) eds. Plotkin & Orenstein. ISBN 0-7216-9688-0.
[87] WO02/02606.
[88] Kalman et al. (1999) Nature Genetics 21:385-389.
[89] Read et al. (2000) Nucleic Acids Res 28:1397-406.
[90] Shirai et al. (2000) J. Infect. Dis. 181(Suppl 3):S524-S527.
[91] WO99/27105.
[92] WO00/27994.
[93] WO00/37494.
[94] WO99/28475.
[95] Ross et al. (2001) Vaccine, 19, 4135.
[96] Sutter et al. (2000) Pediatr. Clin. North. Am., 47, 287.
[97] Zimmerman & Spann (1999) Am. Fam. Physician., 59, 113-118, 125-126.
[98] Dreesen (1997) Vaccine, 15 Suppl, S2.
[99] MMWR Morb. Mortal. Wkly. Rep. (1998), 47, 12, 19.
[100] McMichael (2000) Vaccine, 19 Suppl 1, S101.
[101] WO02/34771.
[102] Dale (1999) Infect. Dis. Clin. North. Am., 13, 227, viii.
[103] Ferretti et al. (2001) PNAS USA, 98, 4658.
[104] WO03/093306.
[105] WO2004/018646.
[106] WO2004/041157.
[107] Ichiman and Yoshida (1981) J. Appl. Bacteriol., 51, 229.
[108] U.S. Pat. No. 4,197,290
[109] Ichiman et al. (1991) J. Appl. Bacteriol., 71, 176.
[110] Robinson & Torres (1997) Seminars in Immunology, 9, 271.
[111] Donnelly et al. (1997) Annu. Rev. Immunol., 15, 617.
[112] Scott-Taylor & Dalgleish (2000) Expert. Opin. Investig. Drugs, 9, 471.
[113] Apostolopoulos & Plebanski (2000) Curr. Opin. Mol. Ther., 2, 441.
[114] Ilan (1999) Curr. Opin. Mol. Ther., 1, 116.
[115] Dubensky et al. (2000) Mol. Med., 6, 723.
[116] Robinson & Pertmer (2000) Adv. Virus Res., 55, 1.
[117] Donnelly et al. (2000) Am. J. Respir. Crit. Care Med., 162, S190.
[118] Davis (1999) Mr. Sinai J. Med., 66, 84.
[119] Gennaro (2000) Remington: The Science and Practice of Pharmacy. 20th edition.
[120] WO03/009869.
[121] Almeida & Alpar (1996) J Drug Targeting, 3, 455.
[122] Agarwal & Mishra (1999) Indian J. Exp. Biol., 37, 6.
[123] WO00/53221.
[124] Jakobsen et al. (2002) Infect. Immun., 70, 1443.
[125] Bergquist et al. (1998) APMIS, 106, 800.
[126] Baudner et al. (2002) Infect. Immun., 70, 4785.
[127] Ugozzoli et al. (2002) J. Infect. Dis., 186, 1358.
[128] Vaccine Design (1995) Powell & Newman, Plenum.
[129] WO00/23105.
[130] WO90/14837.
[131] Podda (2001) Vaccine, 19, 2673.
[132] Frey et al. (2003) Vaccine, 21, 4234.
[133] U.S. Pat. No. 6,299,884.
[134] U.S. Pat. No. 6,451,325.
[135] U.S. Pat. No. 5,057,540.
[136] WO96/33739.
[137] EP-A-0109942.
[138] WO96/11711.
[139] WO00/07621.
[140] Barr et al. (1998) Advanced Drug Delivery Reviews, 32, 247.
[141] Sjolanderet et al. (1998) Advanced Drug Delivery Reviews, 32, 321.
[142] Niikura et al. (2002) Virology, 293, 273.
[143] Lenz et al. (2001) J. Immunol., 166, 5346.
[144] Pinto et al. (2003) J. Infect. Dis., 188, 327.
[145] Gerber et al. (2001) Virol., 75, 4752.
[146] WO03/024480.
[147] WO03/024481.
[148] Gluck et al. (2002) Vaccine, 20, B10.
[149] EP-A-0689454.
[150] Johnson et al. (1999) Bioorg. Med. Chem. Lett., 9, 2273.
[151] Evans et al. (2003) Expert Rev. Vaccines, 2, 219.
[152] Meraldi et al. (2003) Vaccine, 21, 2485.
[153] Pajak et al. (2003) Vaccine, 21, 836.
[154] Kandimalla et al. (2003) Nucleic Acids Research, 31, 2393.
[155] WO02/26757.
[156] WO99/62923.
[157] Krieg (2003) Nature Medicine, 9, 831.
[158] McCluskie et al. (2002) FEMS Immunology and Medical Microbiology, 32, 179.
[159] WO98/40100.
[160] U.S. Pat. No. 6,207,646.
[161] U.S. Pat. No. 6,239,116.
[162] U.S. Pat. No. 6,429,199.
[163] Kandimalla et al. (2003) Biochemical Society Transactions, 31, 654.
[164] Blackwell et al. (2003) J. Immunol., 170, 4061.
[165] Krieg (2002) Trends Immunol., 23, 64.
[166] WO01/95935.
[167] Kandimalla et al. (2003) BBRC, 306, 948.
[168] Bhagat et al. (2003) BBRC, 300, 853.
[169] WO03/035836.
[170] WO95/17211.
[171] WO98/42375.
[172] Beignon et al. (2002) Infect. Immun., 70, 3012.
[173] Pizza et al. (2001) Vaccine, 19, 2534.
[174] Pizza et al. (2000) Int. J. Med. Microbiol., 290, 455.
[175] Scharton-Kersten et al. (2000) Infect. Immun., 68, 5306.
[176] Ryan et al. (1999) Infect. Immun., 67, 6270.
[177] Partidos et al. (1999) Immunol. Lett., 67, 209.
[178] Peppoloni et al. (2003) Expert. Rev. Vaccines, 2, 285.
[179] Pine et al. (2002) J. Control. Release., 85, 263.
[180] Domenighini et al. (1995) Mol. Microbiol., 15, 1165.
[181] WO99/40936.
[182] WO99/44636.
[183] Singh et al. (2001) J. Cont. Release., 70, 267.
[184] WO99/27960.
[185] U.S. Pat. No. 6,090,406.
[186] U.S. Pat. No. 5,916,588.
[187] EP-A-0626169.
[188] WO99/52549.
[189] WO01/21207.
[190] WO01/21152.
[191] Andrianov et al. (1998) Biomaterials, 19, 109.
[192] Payne et al. (1998) Adv. Drug Delivery Review, 31, 185.
[193] Stanley (2002) Clin. Exp. Dermatol., 27, 571.
[194] Jones (2003) Curr. Opin. Investig. Drugs, 4, 214.
[195] WO04/60308.
[196] WO04/64759.
[197] WO99/11241.
[198] WO94/00153.
[199] WO98/57659.
[200] European patent applications 0835318, 0735898 and 0761231.
[201] Gennaro (2000) Remington: The Science and Practice of Pharmacy. 20th edition.
[202] Colowick & Kaplan, Methods In Enzymology, Academic Press, Inc.
[203] Handbook of Experimental Immunology, Vols. I-IV (D. M. Weir and C. C. Blackwell, eds, 1986, Blackwell Scientific Publications).
[204] Sambrook et al. (2001) Molecular Cloning: A Laboratory Manual, 3rd edition (Cold Spring Harbor Laboratory Press).
[205] Handbook of Surface and Colloidal Chemistry (Birdi, K. S. ed., CRC Press, 1997).
[206] Ausubel et al. (eds) (2002) Short protocols in molecular biology, 5th edition (Current Protocols).
[207] Molecular Biology Techniques: An Intensive Laboratory Course, (Ream et al., eds., 1998, Academic Press)
[208] PCR (Introduction to Biotechniques Series), 2nd ed. (Newton & Graham eds., 1997, Springer Verlag)
[209] Bardotti et al. (2009) Vaccine, 26, 2284.
[210] Zhang et al. (1996) Carbohydr. Res., 280, 251.
[211] Ellervik et al. (1998) J. Org. Chem., 63, 9323.
[212] Wang et al. (2005) Tetrahedron, 61, 4313.
[213] Evans et al. (1977) Carbohydr. Res., 54, 105.
[214] Handa et al. (1979) Carbohydr. Res., 73, C1.
[215] Nitz (2000) J. Org. Chem., 65, 3064.
[216] Wu et al. (2007) Org. Biomol. Chem., 5, 3477.
[217] Lay et al. (1998) Carbohydr. Res., 310, 157.
[218] Haag et al. (2007) Eur. Org. Chem., 6016.
[219] Oikawa et al. (1996) Synlett., 1179.
[220] Baumann et al. (2008) Angew. Chem. Int. Ed., 47, 3445.
[221] Mandal et al. (2004) Angew. Chem. Int. Ed., 43, 2557.
[222] Zhang et al. (2008) Chem. Eur. J., 14, 558.
[223] Dubois (1956) Anal. Chem., 28, 250.
[224] Waffenschmidt (1987) Anal. Biochem., 165, 337.

1. A synthetic C. difficile PS-II cell wall saccharide.

2. The saccharide of claim 1, wherein said saccharide is a single molecular species.

3. The saccharide of claim 1, wherein said saccharide is a hexasaccharide or a dodecasaccharide.

4. The saccharide of claim 1, wherein said saccharide lacks a phosphate group at the 6-O-position of the non-reducing terminal saccharide.

5. The saccharide of claim 1, wherein peptidoglycan contamination and/or protein contamination are undetectable.

6. The saccharide of claim 1, wherein said saccharide has the structure of Formula I: 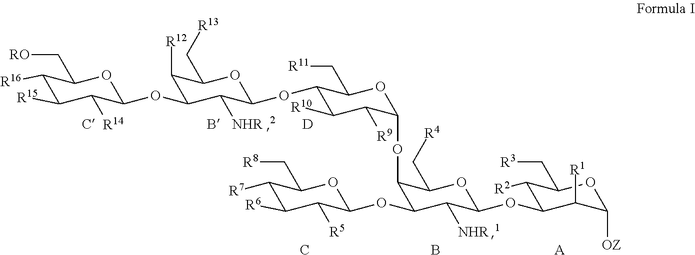
wherein
R is selected from H, PO₃H₂ or an anionic form thereof, acetyl, and a hydroxyl protecting group;
each R¹, R², R³, R⁴, R⁵, R⁶, R⁷, R⁸, R⁹, R¹⁰, R¹¹, R¹², R¹³, R¹⁴, R¹⁵ and R¹⁶ is independently selected from OH and a blocking group;
each R,¹ and R,² is independently selected from H, acetyl, and an amino protecting group;
and
Z is selected from H, a linker and a hydroxyl protecting group.

7. The saccharide of claim 6, wherein
R is PO₃H₂ or an anionic form thereof;
R¹, R², R³, R⁴, R⁵, R⁶, R⁷, R⁸, R⁹, R¹⁰, R¹¹, R¹², R¹³, R¹⁴, R¹⁵ and R¹⁶ are OH;
R,¹ and R,² are acetyl groups; and
Z is a linker.

8. The saccharide of claim 1, wherein the reducing terminus forms a covalent bond with a linker as in Formula IV: 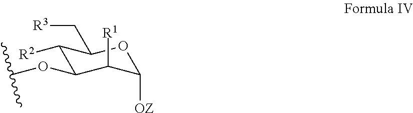
wherein
each R¹, R² and R³ is independently selected from OH and a blocking group; and
Z is a linker.

9. The saccharide of claim 8, wherein R¹, R² and R³ are OH.

10. The saccharide of claim 8, wherein the linker is a 1-aminopropyl group.

11. The saccharide of claim 1, wherein said saccharide is conjugated to a carrier molecule.

12. The saccharide of claim 8, wherein the saccharide is conjugated to a carrier molecule via the linker.

13. A saccharide of Formula II: 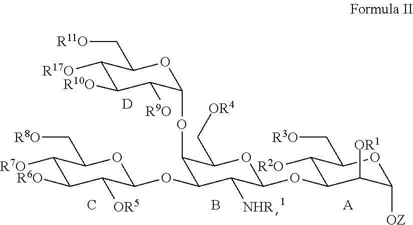
wherein
each R¹, R², R³, R⁴, R⁵, R⁶, R⁷, R⁸, R⁹, R¹⁰, R¹¹, and R¹⁷ is independently selected from OH and a blocking group;
R,¹ is selected from H, acetyl, and an amino protecting group; and
Z is selected from H, a linker and a hydroxyl protecting group.

14. The saccharide of claim 13, wherein
R¹, R², R³, R⁴, R⁵, R⁶R⁷, R⁸, R⁹, R¹⁰, R¹¹, and R¹⁷ are OH;
R,¹ is an acetyl; and
Z is a linker.

15. A saccharide of Formula III: 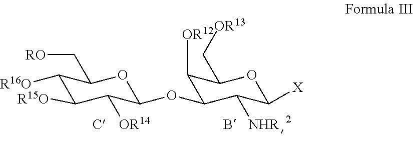
wherein
R is selected from H, PO₃H₂ or an anionic form thereof, acetyl, and a hydroxyl protecting group;
each R¹², R¹³, R¹⁴, R¹⁵ and R¹⁶ is independently selected from OH and a blocking group;
R,² is independently selected from H, acetyl, and an amino protecting group; and
X is an activating group.

16. The saccharide of claim 15, wherein
R¹², R¹³, R¹⁴, R¹⁵ and R¹⁶ are OH;
R,² is an acetyl;
R is PO₃H₂ or an anionic form thereof; and
X is SPh.

17. A composition comprising C. difficile PS-II cell wall saccharide, wherein the composition comprises saccharide and (a) a level of peptidoglycan contamination that is less than 5% by weight peptidoglycan relative to the total weight of the saccharide; and/or (b) a level of protein contamination that is less than 5% by weight protein relative to the total weight of the saccharide.

18. A pharmaceutical composition comprising a saccharide according to claim 1 in combination with a pharmaceutically acceptable carrier.

19. The pharmaceutical composition of claim 18, further comprising an adjuvant.

20. A process for preparing a pharmaceutical composition, comprising the steps of mixing a saccharide of claim 1 with a pharmaceutically acceptable carrier.

21. A method for raising an immune response in a mammal, comprising administering a pharmaceutical composition of claim 18 to the mammal.

22. A method of making a saccharide of claim 1.

23. The method of claim 22, wherein the saccharide is made in vitro.

24. The method of claim 22, wherein the method comprises reacting an intermediate according to Formula II: 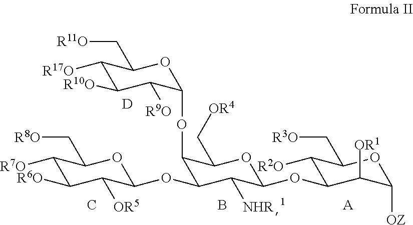
wherein
R¹, R², R³, R⁴, R⁵, R⁶, R⁷, R⁸, R⁹, R¹⁰, R¹¹, and R¹⁷ are OH;
R,¹ is acetyl; and
Z is a linker,
with an intermediate according to Formula III: 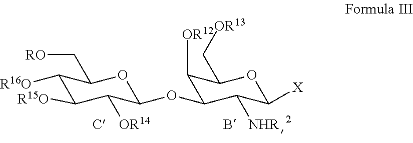
wherein
R is PO₃H₂ or an anionic form thereof;
R¹², R¹³, R¹⁴, R¹⁵ and R¹⁶ are OH;
R,² is an acetyl; and
X is an activating group.

25. A process for purifying C. difficile PS-II saccharide from C. difficile bacterial cells, wherein said process comprises the step of (a) inactivating the bacterial cells with acid.

26. The process of claim 25, wherein the acid is acetic acid.

27. The process of claim 25, wherein the process further comprises one or more of the following steps:
(b) neutralisation;
(c) centrifugation of the bacterial cells and collection of the saccharide-containing supernatant;
(d) fractionation;
(e) treatment of the saccharide with RNase and/or DNase;
(f) treatment of the saccharide with mutanolysin;
(g) anion exchange chromatography;
(h) concentration of the saccharide;
(i) cation exchange chromatography; and
(j) depolymerisation to form an oligosaccharide.

28. The process of claim 25, wherein the process provides a composition comprising saccharide and (a) a level of peptidoglycan contamination that is less than 5% by weight peptidoglycan relative to the total weight of the saccharide; and/or (b) a level of protein contamination that is less than 5% by weight protein relative to the total weight of the saccharide.
